(19) United States
(12) Patent Application Publication
(10) Pub. No.: US 2024/0174753 A1
(43) Pub. Date: May 30, 2024

(54) FUSION PROTEINS, PHARMACEUTICAL COMPOSITIONS, AND THERAPEUTIC APPLICATIONS

(71) Applicant: Anwita Biosciences, Inc., San Carlos, CA (US)

(72) Inventors: Ziyang Zhong, Belmont, CA (US); Fan Ye, Mountain View, CA (US); Matthew Siegel, Menlo Park, CA (US); Jianing Huang, San Mateo, CA (US)

(21) Appl. No.: 18/552,381

(22) PCT Filed: Mar. 31, 2022
(86) PCT No.: PCT/US22/22697

§ 371 Date: Sep. 25, 2023

Related U.S. Application Data
(60) Provisional application No. 63/169,132, filed on Mar. 31, 2021.

(51) Int. Cl. C07K 16/28; A61K 39/00; A61P 35/00; C07K 14/55

(52) U.S. Cl. CPC C07K 16/2818; A61P 35/00; C07K 14/55; A61K 2039/505; C07K 2317/21; C07K 2317/569; C07K 2317/73; C07K 2317/94; C07K 2319/30

(57) ABSTRACT

Provided herein are a fusion protein comprising an interleukin-2 domain, a programmed cell death-1 binding domain, and a half-life-extension domain; and a pharmaceutical composition thereof. Also provided herein is a method of their use for treating, preventing, or ameliorating one or more symptoms of a proliferative disease.

### CROSS REFERENCE TO RELATED APPLICATION

This application claims the benefit of the priority of U.S. Provisional Application No. 63/169,132, filed Mar. 31, 2021; the disclosure of which is incorporated herein by reference in its entirety.

### FIELD

Provided herein are a fusion protein comprising an interleukin-2 domain, a programmed cell death-1 binding domain, and a half-life-extension domain; and a pharmaceutical composition thereof. Also provided herein is a method of their use for treating, preventing, or ameliorating one or more symptoms of a proliferative disease.

### REFERENCE TO A SEQUENCE LISTING

The present specification is being filed with a Sequence Listing in Computer Readable Form (CRF), which is entitled 216A013WO01_SEQ_LIST_ST25.txt of 360,545 bytes in size and created Mar. 30, 2022; the content of which is incorporated herein by reference in its entirety.

### BACKGROUND

Dysregulation of the host immune system is one important immune resistance mechanism for cancer. Hanahan and Weinberg, Cell 2011, 144, 646-74; Pardoll, Nat. Rev. Cancer 2012, 12, 252-64. One class of immunotherapy is agents targeting specific checkpoint proteins that play critical roles in regulating T-cell activation and proliferation. Waldman et al., Nat. Rev. Immunol. 2020, 20, 651-68. These proteins function as co-receptors on the surfaces of T-cells to help regulate T-cell responses following T-cell activation. Wolchok et al., Cancer J. 2010, 16, 311-17. The two best characterized checkpoint proteins are cytotoxic T-lymphocyte antigen 4 (CTLA-4) and programmed cell death-1 (PD-1), both serve as negative regulators of T-cell activation. Waldman et al., Nat. Rev. Immunol. 2020, 20, 651-68. T-cell activation induces expression of CTLA-4 on T lymphocytes, thereby inhibits further T-cell activation and proliferation. PD-1 expression is induced when T-cell become activated. Pardoll, Nat. Rev. Cancer 2012, 12, 252-64. Immune checkpoint blockade removes such inhibitory signals and unleashes antitumor immune responses. Id.; Sharma and Allison, Science 2015, 348, 56-61. Ipilimumab, a CTLA-4 blocking antibody, was the first immune checkpoint inhibitor approved by the FDA for cancer treatment. Id. Several anti-PD-1 antibodies have since been approved for cancer treatment. Gong et al., J. Immunother. Cancer 2018, 6, 8. While immunotherapy has been a major advance in cancer treatment, up to 85 percent of patients whose cancer is treated with checkpoint inhibitors do not benefit. Haslam and Prasad, JAMA Netw. Open 2019, 2, e192535.

An interleukin-2 (IL-2) is a pleiotropic cytokine that orchestrates the proliferation, survival, and function of both immune effector (Teff) cells and regulatory T (Treg) cells to maintain immune homeostasis. Bluestone, N. Engl. J. Med. 2011, 365, 2129-31; Boyman et al., Nat. Rev. Immunol. 2012, 12, 180-90. The IL-2 drives T-cell growth, augments natural killer (NK) cytolytic activity, induces the differentiation of regulatory T (Treg) cells, and mediates activation-induced cell death. Liao et al., Curr. Opin. Immunol. 2011, 23, 598-604.

An interleukin-2 receptor (IL-2R) exists in three different forms generated from three different interleukin-2 receptor chains: α chain (IL-2Rα or CD25), β chain (IL-2Rβ or CD122), and γ chain (IL-2Rγ, γ_{c}, or CD132). Wang et al., Science 2005, 310, 1159-63. The IL-2 binds the IL-2Rα with a low affinity (K_{d}≈10 nM). Id. From a crystal structure of a quaternary IL-2 signaling complex, fifteen amino acid residues (K35, T37, R38, T41, F42, K43, F44, Y45, E61, E62, K64, P65, E68, L72, and Y107) on the IL-2 are identified as interface residues between the IL-2 and IL-2Rα. Stauber et al., Proc. Nat. Acad. Sci. U.S.A. 2006, 103, 2788-93. The IL-2 binds a heterodimeric complex of the IL-2Rβ and IL-2Rγ, expressed on memory T cells and NK cells, with an intermediate affinity (K_{d}≈1 nM). Wang et al., Science 2005, 310, 1159-63. The IL-2 binds a heterotrimeric complex of the IL-2Rα, IL-2Rβ, and IL-2Rγ, expressed on Treg cells, with a high affinity (K_{d}≈10 pM). Id. The IL-2 binds the IL-2Rβ alone with a dissociation constant (K_{d}) of about 100 nM. Id. The IL-2Rα by itself has no signal-transducing activity. Id. The IL-2 signals through the intermediate-affinity heterodimeric IL-2Rβ/γ complex or the high-affinity heterotrimeric IL-2Rα/β/γ complex. Liao et al., Curr. Opin. Immunol. 2011, 23, 598-604. The binding of the IL-2 to the intermediate-affinity heterodimeric IL-2Rβ/γ complex leads to the activation and proliferation of immunostimulatory Teff cells, while the binding of the IL-2 to the high-affinity heterotrimeric IL-2Rα/β/γ complex results in the activation and proliferation of immunosuppressive Treg cells. Malek et al., Immunity 2010, 33, 153-65; Bluestone, N. Engl. J. Med. 2011, 365, 2129-31; Boyman et al., Nat. Rev. Immunol. 2012, 12, 180-90; Spangler et al., Annu. Rev. Immunol. 2015, 33, 139-67. This dual opposing functions of immunostimulation and immunosuppression pose a major challenge in developing the IL-2 as a safe and effective therapeutic agent. Skrombolas et al., Expert Rev. Clin. Immunol. 2014, 10, 207-17; Abbas et al., Sci. Immunol. 2018, 3, eaat1482.

Aldesleukin, a recombinant human IL-2, was approved by the FDA for metastatic renal cell carcinoma in 1992 and for metastatic melanoma in 1998. Rosenberg, J. Immunol. 2014, 192, 5451-8. Patients with metastatic melanoma or renal cancer experience a 5 to 10% rate of complete cancer regression, with an additional 10% experiencing a partial regression. Atkins et al., J. Clin. Oncol. 1999, 17, 2105-16; Klapper et al., Cancer 2008, 113, 293-301. Approximately 70% of complete responders to the IL-2 therapy do not recur. Rosenberg, Sci. Transl. Med. 2012, 4, 127ps8. However, the success of the IL-2 as an immunotherapy for cancer has been hampered by its severe toxicities and limited efficacy. One major limiting factor for its efficacy as an anticancer agent is immunosuppression resulting from the IL-2-driven preferential expansion of Treg cells. Abbas et al., Sci. Immunol. 2018, 3, eaat1482. Moreover, for the IL-2 to be effective in cancer treatment, a high dose therapeutic schedule is required. Bluestone, N. Engl. J. Med. 2011, 365, 2129-31; Abbas et al., Sci. Immunol. 2018, 3, eaat1482. This dosing regimen, however, causes vascular leak syndrome and results in the limited application of IL-2 in cancer treatment. Abbas et al., Sci. Immunol. 2018, 3, eaat1482.

Despite the advances in cancer treatment, cancer remains a major worldwide public health problem. It was estimated that there will be 1,898,160 new cancer cases diagnosed and 608,570 cancer deaths in the US alone in 2021. Cancer Facts & Figures. 2021. Therefore, there is a need for an effective therapy for cancer treatment.

### SUMMARY OF THE DISCLOSURE

Provided herein is a fusion protein comprising an interleukin-2 (IL-2) domain, a programmed cell death-1 (PD-1) binding domain, and a half-life-extension domain.

Also provided herein is a fusion protein comprising an IL-2 domain, a PD-1 binding domain, and an albumin binding domain.

Additionally, provided herein is a fusion protein comprising an IL-2 domain, a PD-1 binding domain, an albumin binding domain, and optionally first and second peptide linkers; wherein the amino terminus (N-terminus) of the IL-2 domain is connected to the carboxyl terminus (C-terminus) of the albumin binding domain directly or via the first peptide linker, and the N-terminus of the albumin binding domain is connected to the C-terminus of the PD-1 binding domain directly or via the second peptide linker.

Furthermore, provided herein is a fusion protein comprising an IL-2 domain, a PD-1 binding domain, an albumin binding domain, and optionally first and second peptide linkers; wherein the C-terminus of the IL-2 domain is connected to the N-terminus of the albumin binding domain directly or via the first peptide linker, and the C-terminus of the albumin binding domain is connected to the N-terminus of the PD-1 binding domain directly or via the second peptide linker.

Provided herein is a fusion protein comprising an IL-2 domain, a PD-1 binding domain, an albumin binding domain, and optionally first and second peptide linkers; wherein the N-terminus of the IL-2 domain is connected to the C-terminus of the PD-1 binding domain directly or via the first peptide linker, and the N-terminus of the PD-1 binding domain is connected to the C-terminus of the albumin binding domain directly or via the second peptide linker.

Provided herein is a fusion protein comprising an IL-2 domain, a PD-1 binding domain, an albumin binding domain, and optionally first and second peptide linkers; wherein the C-terminus of the IL-2 domain is connected to the N-terminus of the PD-1 binding domain directly or via the first peptide linker, and the C-terminus of the PD-1 binding domain is connected to the N-terminus of the albumin binding domain directly or via the second peptide linker.

Provided herein is a fusion protein comprising an IL-2 domain, a PD-1 binding domain, an albumin binding domain, and optionally first and second peptide linkers; wherein the N-terminus of the PD-1 binding domain is connected to the C-terminus of the IL-2 domain directly or via the first peptide linker, and the N-terminus of the IL-2 domain is connected to the C-terminus of the albumin binding domain directly or via the second peptide linker.

Provided herein is a fusion protein comprising an IL-2 domain, a PD-1 binding domain, an albumin binding domain, and optionally first and second peptide linkers; wherein the C-terminus of the PD-1 binding domain is connected to the N-terminus of the IL-2 domain directly or via the first peptide linker, and the C-terminus of the IL-2 domain is connected to the N-terminus of the albumin binding domain directly or via the second peptide linker.

Provided herein is a fusion protein comprising an IL-2 domain, a PD-1 binding domain, and a half-life-extension domain comprising a fragment crystallizable (Fc) domain.

Provided herein is a fusion protein comprising an IL-2 domain, first and second PD-1 binding domains, a half-life-extension domain that comprises an Fc domain having first and second peptide chains, and optionally a peptide linker; wherein the N-terminus of the IL-2 domain is connected to the C-terminus of the first peptide chain of the Fc domain directly or via the peptide linker.

Provided herein is a fusion protein comprising an IL-2 domain, first and second PD-1 binding domains, a half-life-extension domain that comprises an Fc, and optionally a peptide linker; wherein the C-terminus of the IL-2 domain is connected to an N-terminus of the first PD-1 binding domain directly or via the peptide linker.

Provided herein is a fusion protein comprising first and second IL-2 domains, first and second PD-1 binding domains, a half-life-extension domain that comprises an Fc domain having first and second peptide chains, and optionally first and second peptide linkers; wherein the N-terminus of the first IL-2 domain is connected to the C-terminus of the first peptide chain of the Fc domain directly or via the first peptide linker; and the N-terminus of the second IL-2 domain is connected to the C-terminus of the second peptide chain of the Fc domain directly or via the second peptide linker.

Provided herein is a fusion protein comprising an IL-2 domain and an anti-PD-1 antibody.

Provided herein is a fusion protein comprising an IL-2 domain, an anti-PD-1 antibody comprising light and heavy chains, and optionally a peptide linker, wherein the N-terminus of the IL-2 domain is connected to a C-terminus of the heavy chains directly or via the peptide linker.

Provided herein is a fusion protein comprising first and second IL-2 domains, an anti-PD-1 antibody comprising two light chains and first and second heavy chains, and optionally first and second peptide linkers, wherein the N-terminus of the first IL-2 domain is connected to the C-terminus of the first heavy chain directly or via the first peptide linker; and the N-terminus of the second IL-2 domain is connected to the C-terminus of the second heavy chain directly or via the second peptide linker.

Provided herein is a fusion protein comprising an IL-2 domain, an anti-PD-1 antibody comprising light and heavy chains, and optionally a peptide linker, wherein the C-terminus of the IL-2 domain is connected to an N-terminus of the heavy chains directly or via the peptide linker.

Provided herein is a fusion protein comprising an IL-2 domain, an anti-PD-1 antibody comprising light and heavy chains, and optionally a peptide linker, wherein the C-terminus of the IL-2 domain is connected to an N-terminus of the light chains directly or via the peptide linker.

Provided herein is a pharmaceutical composition comprising a fusion protein provided herein and a pharmaceutically acceptable excipient.

Provided herein is a method of treating, preventing, or ameliorating one or more symptoms of a disorder, disease, or condition mediated by a PD-1 in a subject, comprising administering to the subject in need thereof a therapeutically effective amount of a fusion protein provided herein.

Provided herein is a method of treating, preventing, or ameliorating one or more symptoms of a disorder, disease, or condition mediated by an IL-2 in a subject, comprising administering to the subject in need thereof a therapeutically effective amount of a fusion protein provided herein.

Provided herein is a method of treating, preventing, or ameliorating one or more symptoms of a proliferative disease in a subject, comprising administering to the subject in need thereof a therapeutically effective amount of a fusion protein provided herein.

Provided herein is a method of inhibiting the growth of a cell, comprising contacting the cell with an effective amount of a fusion protein provided herein.

Provided herein is a method of activating an immune effector cell, comprising contacting the cell with an effective amount of a fusion protein provided herein.

### BRIEF DESCRIPTION OF THE DRAWINGS

FIG. 1 shows the configuration of an exemplary fusion protein that comprises an anti-PD-1 antibody having two PD-1 binding domains (e.g., two pairs of heavy chain (VH) and light chain variable (VL) regions) and an Fc domain functioning as a half-life-extension domain, and one or two IL-2 domains; wherein the N-terminus of an IL-2 domain is connected via a peptide linker to the C-terminus of a heavy chain of the anti-PD-1 antibody.

FIG. 2 shows the configuration of an exemplary fusion protein that comprises an anti-PD-1 antibody having two PD-1 binding domains (e.g., two V_{H}H single domain antibodies) and an Fc domain functioning as a half-life-extension domain, and one or two IL-2 domains; wherein the N-terminus of an IL-2 domain is connected via a peptide linker to the C-terminus of a heavy chain of the anti-PD-1 antibody.

FIG. 3 shows the configuration of an exemplary fusion protein that comprises an anti-PD-1 domain comprising a single-chain variable fragment (scFv) or V_{H}H single domain antibody, an IL-2 domain, an anti-HSA V_{H}H single domain antibody as a half-life extension domain, and first and second peptide linkers; wherein the N-terminus of the IL-2 domain is connected to the C-terminus of the anti-HSA V_{H}H single domain antibody via the first peptide linker, and the N-terminus of the anti-HSA V_{H}H single domain antibody is connected to the C-terminus of the anti-PD-1 scFv or V_{H}H single domain antibody.

FIG. 4 shows the antitumor effects of anti-PD-1/IL-2 fusion proteins in a xenograft mouse model for colorectal cancer (HT-29 cells).

### DETAILED DESCRIPTION

To facilitate understanding of the disclosure set forth herein, a number of terms are defined below.

Generally, the nomenclature used herein and the laboratory procedures in biochemistry, biology, cell biology, immunology, molecular biology, and pharmacology described herein are those well-known and commonly employed in the art. Unless defined otherwise, all technical and scientific terms used herein generally have the same meaning as commonly understood by one of ordinary skill in the art to which this disclosure belongs.

The term “subject” refers to an animal, including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject. In one embodiment, the subject is a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The terms “alleviate” and “alleviating” refer to easing or reducing one or more symptoms (e.g., pain) of a disorder, disease, or condition. The terms can also refer to reducing adverse effects associated with an active ingredient. Sometimes, the beneficial effects that a subject derives from a prophylactic or therapeutic agent do not result in a cure of the disorder, disease, or condition.

The term “contacting” or “contact” is meant to refer to bringing together of a therapeutic agent and cell or tissue such that a physiological and/or chemical effect takes place as a result of such contact. Contacting can take place in vitro, ex vivo, or in vivo. In one embodiment, a therapeutic agent is contacted with a cell in cell culture (in vitro) to determine the effect of the therapeutic agent on the cell. In another embodiment, the contacting of a therapeutic agent with a cell or tissue includes the administration of a therapeutic agent to a subject having the cell or tissue to be contacted.

The term “therapeutically effective amount” or “effective amount” is meant to include the amount of a compound (e.g., a fusion protein) that, when administered, is sufficient to prevent development of, or alleviate to some extent, one or more of the symptoms of the disorder, disease, or condition being treated. The term “therapeutically effective amount” or “effective amount” also refers to the amount of a compound that is sufficient to elicit a biological or medical response of a biological molecule (e.g., a protein, enzyme, RNA, or DNA), cell, tissue, system, animal, or human, which is being sought by a researcher, veterinarian, medical doctor, or clinician.

The term “IC₅₀” or “EC₅₀” refers to an amount, concentration, or dosage of a compound (e.g., a fusion protein) that is required for 50% inhibition of a maximal response in an assay that measures such a response.

The term “pharmaceutically acceptable carrier,” “pharmaceutically acceptable excipient,” “physiologically acceptable carrier,” or “physiologically acceptable excipient” refers to a pharmaceutically acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of a subject (e.g., a human or an animal) without excessive toxicity, irritation, allergic response, immunogenicity, or other problems or complications, commensurate with a reasonable benefit/risk ratio. See, Remington: The Science and Practice of Pharmacy, 23rd ed.; Adejare Ed.; Academic Press: 2020; Handbook of Pharmaceutical Excipients, 9th ed.; Sheskey et al., Eds.; The Pharmaceutical Press: 2020; Handbook of Pharmaceutical Additives, 3rd ed.; Ash and Ash Eds.; Synapse Information Resources, Inc.: 2007; Pharmaceutical Preformulation and Formulation, 2nd ed.; Gibson Ed.; CRC Press: 2009.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

The terms “substantially pure” and “substantially homogeneous,” when referring to a compound (e.g. a fusion protein), mean sufficiently homogeneous to appear free of readily detectable impurities as determined by standard analytical methods used by one of ordinary skill in the art, including, but not limited to, gel electrophoresis, high performance liquid chromatography (HPLC), and mass spectrometry (MS); or sufficiently pure such that further purification would not detectably alter the physical, chemical, biological, and/or pharmacological properties, such as enzymatic and biological activities, of the compound. In certain embodiments, “substantially pure” or “substantially homogeneous” refers to a collection of molecules, wherein at least about 50%, at least about 70%, at least about 80%, at least about 90%, at least about 95%, at least about 98%, at least about 99%, or at least about 99.5% by weight of the molecules are a single compound as determined by a standard analytical method.

### Fusion Proteins

In one embodiment, provided herein is a fusion protein comprising an interleukin-2 (IL-2) domain, a programmed cell death-1 (PD-1) binding domain, and a half-life-extension domain. In certain embodiments, the IL-2 domain causes the fusion protein to signal through a receptor comprising CD122 (IL-2Rβ/IL-15Rβ) and CD132 (IL-2Rγ) subunits.

In another embodiment, provided herein is a fusion protein comprising an IL-2 domain, a PD-1 binding domain, and a half-life-extension domain; wherein the IL-2 domain causes the fusion protein to signal through a receptor consisting of CD122 and CD132 subunits.

In certain embodiments, the half-life-extension domain extends the half-life of the IL-2 domain in vivo as compared to the corresponding free IL-2. In certain embodiments, the half-life-extension domain extends the half-life of the interleukin-2 domain in vivo as compared to a wild-type interleukin-2 of SEQ ID NO: 1, 2, 3, 4, or 5.

In certain embodiments, the half-life-extension domain is an albumin binding domain. In certain embodiments, the half-life-extension domain comprises a fragment crystallizable (Fc) domain. In certain embodiments, the half-life-extension domain is a constant region (C_{H}1, C_{H}2, and C_{H}3) of a heavy chain of an antibody. In certain embodiments, the half-life-extension domain is a Fc domain.

In one embodiment, provided herein is a fusion protein comprising an IL-2 domain, a PD-1 binding domain, and an albumin binding domain. In certain embodiments, the IL-2 domain causes the fusion protein to signal through a receptor comprising CD122 and CD132 subunits.

In another embodiment, provided herein is a fusion protein comprising an IL-2 domain, a PD-1 binding domain, and an albumin binding domain; wherein the IL-2 domain causes the fusion protein to signal through a receptor consisting of CD122 and CD132 subunits.

In yet another embodiment, provided herein is a fusion protein comprising an IL-2 domain, a PD-1 binding domain, an albumin binding domain, and optionally first and second peptide linkers; wherein the amino terminus (N-terminus) of the IL-2 domain is connected to the carboxyl terminus (C-terminus) of the albumin binding domain directly or via the first peptide linker, and the N-terminus of the albumin binding domain is connected to the C-terminus of the PD-1 binding domain directly or via the second peptide linker.

In one embodiment, the fusion protein provided herein comprises an IL-2 domain, a PD-1 binding domain, and an albumin binding domain; wherein the N-terminus of the IL-2 domain is connected to the C-terminus of the albumin binding domain directly, and the N-terminus of the albumin binding domain is connected to the C-terminus of the PD-1 binding domain directly.

In another embodiment, the fusion protein provided herein comprises an IL-2 domain, a PD-1 binding domain, an albumin binding domain, and a peptide linker; wherein the N-terminus of the IL-2 domain is connected to the C-terminus of the albumin binding domain directly, and the N-terminus of the albumin binding domain is connected to the C-terminus of the PD-1 binding domain via the peptide linker; or wherein the N-terminus of the IL-2 domain is connected to the C-terminus of the albumin binding domain via the peptide linker, and the N-terminus of the albumin binding domain is connected to the C-terminus of the PD-1 binding domain directly.

In yet another embodiment, the fusion protein provided herein comprises an IL-2 domain, a PD-1 binding domain, an albumin binding domain, and first and second peptide linkers; wherein the N-terminus of the IL-2 domain is connected to the C-terminus of the albumin binding domain via the first peptide linker, and the N-terminus of the albumin binding domain is connected to the C-terminus of the PD-1 binding domain via the second peptide linker.

In yet another embodiment, provided herein is a fusion protein comprising an IL-2 domain, a PD-1 binding domain, an albumin binding domain, and optionally first and second peptide linkers; wherein the C-terminus of the IL-2 domain is connected to the N-terminus of the albumin binding domain directly or via the first peptide linker, and the C-terminus of the albumin binding domain is connected to the N-terminus of the PD-1 binding domain directly or via the second peptide linker.

In one embodiment, the fusion protein provided herein comprises an IL-2 domain, a PD-1 binding domain, and an albumin binding domain; wherein the C-terminus of the IL-2 domain is connected to the N-terminus of the albumin binding domain directly, and the C-terminus of the albumin binding domain is connected to the N-terminus of the PD-1 binding domain directly.

In another embodiment, the fusion protein provided herein comprises an IL-2 domain, a PD-1 binding domain, an albumin binding domain, and a peptide linker; wherein the C-terminus of the IL-2 domain is connected to the N-terminus of the albumin binding domain directly, and the C-terminus of the albumin binding domain is connected to the N-terminus of the PD-1 binding domain via the peptide linker; or wherein the C-terminus of the IL-2 domain is connected to the N-terminus of the albumin binding domain via the peptide linker; and the C-terminus of the albumin binding domain is connected to the N-terminus of the PD-1 binding domain directly.

In yet another embodiment, the fusion protein provided herein comprises an IL-2 domain, a PD-1 binding domain, an albumin binding domain, and first and second peptide linkers; wherein the C-terminus of the IL-2 domain is connected to the N-terminus of the albumin binding domain via the first peptide linker; and the C-terminus of the albumin binding domain is connected to the N-terminus of the PD-1 binding domain via the second peptide linker.

In yet another embodiment, provided herein is a fusion protein comprising an IL-2 domain, a PD-1 binding domain, an albumin binding domain, and optionally first and second peptide linkers; wherein the N-terminus of the IL-2 domain is connected to the C-terminus of the PD-1 binding domain directly or via the first peptide linker, and the N-terminus of the PD-1 binding domain is connected to the C-terminus of the albumin binding domain directly or via the second peptide linker.

In one embodiment, the fusion protein provided herein comprises an IL-2 domain, a PD-1 binding domain, and an albumin binding domain; wherein the N-terminus of the IL-2 domain is connected to the C-terminus of the PD-1 binding domain directly, and the N-terminus of the PD-1 binding domain is connected to the C-terminus of the albumin binding domain directly.

In another embodiment, the fusion protein provided herein comprises an IL-2 domain, a PD-1 binding domain, an albumin binding domain, and a peptide linker; wherein the N-terminus of the IL-2 domain is connected to the C-terminus of the PD-1 binding domain directly, and the N-terminus of the PD-1 binding domain is connected to the C-terminus of the albumin binding domain via the peptide linker; or wherein the N-terminus of the IL-2 domain is connected to the C-terminus of the PD-1 binding domain via the peptide linker, and the N-terminus of the PD-1 binding domain is connected to the C-terminus of the albumin binding domain directly.

In yet another embodiment, the fusion protein provided herein comprises an IL-2 domain, a PD-1 binding domain, an albumin binding domain, and first and second peptide linkers; wherein the N-terminus of the IL-2 domain is connected to the C-terminus of the PD-1 binding domain via the first peptide linker, and the N-terminus of the PD-1 binding domain is connected to the C-terminus of the albumin binding domain via the second peptide linker.

In yet another embodiment, provided herein is a fusion protein comprising an IL-2 domain, a PD-1 binding domain, an albumin binding domain, and optionally first and second peptide linkers; wherein the C-terminus of the IL-2 domain is connected to the N-terminus of the PD-1 binding domain directly or via the first peptide linker, and the C-terminus of the PD-1 binding domain is connected to the N-terminus of the albumin binding domain directly or via the second peptide linker.

In one embodiment, the fusion protein provided herein comprises an IL-2 domain, a PD-1 binding domain, and an albumin binding domain; wherein the C-terminus of the IL-2 domain is connected to the N-terminus of the PD-1 binding domain directly, and the C-terminus of the PD-1 binding domain is connected to the N-terminus of the albumin binding domain directly.

In another embodiment, the fusion protein provided herein comprises an IL-2 domain, a PD-1 binding domain, an albumin binding domain, and a peptide linker; wherein the C-terminus of the IL-2 domain is connected to the N-terminus of the PD-1 binding domain directly, and the C-terminus of the PD-1 binding domain is connected to the N-terminus of the albumin binding domain via the peptide linker; or wherein the C-terminus of the IL-2 domain is connected to the N-terminus of the PD-1 binding domain via the peptide linker, and the C-terminus of the PD-1 binding domain is connected to the N-terminus of the albumin binding domain directly.

In yet another embodiment, the fusion protein provided herein comprises an IL-2 domain, a PD-1 binding domain, an albumin binding domain, and first and second peptide linkers; wherein the C-terminus of the IL-2 domain is connected to the N-terminus of the PD-1 binding domain via the first peptide linker, and the C-terminus of the PD-1 binding domain is connected to the N-terminus of the albumin binding domain via the second peptide linker.

In yet another embodiment, provided herein is a fusion protein comprising an IL-2 domain, a PD-1 binding domain, an albumin binding domain, and optionally first and second peptide linkers; wherein the N-terminus of the PD-1 binding domain is connected to the C-terminus of the IL-2 domain directly or via the first peptide linker, and the N-terminus of the IL-2 domain is connected to the C-terminus of the albumin binding domain directly or via the second peptide linker.

In one embodiment, the fusion protein provided herein comprises an IL-2 domain, a PD-1 binding domain, and an albumin binding domain; wherein the N-terminus of the PD-1 binding domain is connected to the C-terminus of the IL-2 domain directly, and the N-terminus of the IL-2 domain is connected to the C-terminus of the albumin binding domain directly.

In another embodiment, the fusion protein provided herein comprises an IL-2 domain, a PD-1 binding domain, an albumin binding domain, and a peptide linker; wherein the N-terminus of the PD-1 binding domain is connected to the C-terminus of the IL-2 domain directly, and the N-terminus of the IL-2 domain is connected to the C-terminus of the albumin binding domain via the peptide linker; or wherein the N-terminus of the PD-1 binding domain is connected to the C-terminus of the IL-2 domain via the peptide linker, and the N-terminus of the IL-2 domain is connected to the C-terminus of the albumin binding domain directly.

In yet another embodiment, the fusion protein provided herein comprises an IL-2 domain, a PD-1 binding domain, an albumin binding domain, and first and second peptide linkers; wherein the N-terminus of the PD-1 binding domain is connected to the C-terminus of the IL-2 domain via the first peptide linker, and the N-terminus of the IL-2 domain is connected to the C-terminus of the albumin binding domain via the second peptide linker.

In still another embodiment, provided herein is a fusion protein comprising an IL-2 domain, a PD-1 binding domain, an albumin binding domain, and optionally first and second peptide linkers; wherein the C-terminus of the PD-1 binding domain is connected to the N-terminus of the IL-2 domain directly or via the first peptide linker, and the C-terminus of the IL-2 domain is connected to the N-terminus of the albumin binding domain directly or via the second peptide linker.

In one embodiment, the fusion protein provided herein comprises an IL-2 domain, a PD-1 binding domain, and an albumin binding domain; wherein the C-terminus of the PD-1 binding domain is connected to the N-terminus of the IL-2 domain directly, and the C-terminus of the IL-2 domain is connected to the N-terminus of the albumin binding domain directly.

In another embodiment, the fusion protein provided herein comprises an IL-2 domain, a PD-1 binding domain, an albumin binding domain, and a peptide linker; wherein the C-terminus of the PD-1 binding domain is connected to the N-terminus of the IL-2 domain directly, and the C-terminus of the IL-2 domain is connected to the N-terminus of the albumin binding domain via the peptide linker; or wherein the C-terminus of the PD-1 binding domain is connected to the N-terminus of the IL-2 domain via the peptide linker, and the C-terminus of the IL-2 domain is connected to the N-terminus of the albumin binding domain directly.

In yet another embodiment, the fusion protein provided herein comprises an IL-2 domain, a PD-1 binding domain, an albumin binding domain, and first and second peptide linkers; wherein the C-terminus of the PD-1 binding domain is connected to the N-terminus of the IL-2 domain via the first peptide linker, and the C-terminus of the IL-2 domain is connected to the N-terminus of the albumin binding domain via the second peptide linker.

In one embodiment, the albumin binding domain is an antibody or a fragment thereof that binds to an albumin. In another embodiment, the albumin binding domain is an antibody or a fragment thereof that binds to a human serum albumin (HSA).

In certain embodiments, the fusion protein comprising an albumin binding domain binds to an HSA with a K_{d} ranging from about 10 pM to about 1,000 nM, from about 1 to about 500 nM, from about 1 to about 200 nM, or from about 1 to about 100 nM. In certain embodiments, the fusion protein comprising an albumin binding domain binds to an HSA with a K_{d} ranging from about 10 pM to about 1,000 nM. In certain embodiments, the fusion protein comprising an albumin binding domain binds to an HSA with a K_{d} ranging from about 1 to about 500 nM. In certain embodiments, the fusion protein comprising an albumin binding domain binds to an HSA with a K_{d} ranging from about 1 to about 200 nM. In certain embodiments, the fusion protein comprising an albumin binding domain binds to an HSA with a K_{d} ranging from about 1 to about 100 nM.

In one embodiment, the albumin binding domain is an antibody or a fragment thereof, comprising (i) complementarity determining region 1 (CDR1) of SEQ ID NO: 83, complementarity determining region 2 (CDR2) of SEQ ID NO: 84, and complementarity determining region 3 (CDR3) of SEQ ID NO: 85; or (ii) CDR1 of SEQ ID NO: 91, CDR2 of SEQ ID NO: 92, and CDR3 of SEQ ID NO: 93. In another embodiment, the albumin binding domain comprises CDR1 of SEQ ID NO: 83, CDR2 of SEQ ID NO: 84, and CDR3 of SEQ ID NO: 85. In yet another embodiment, the albumin binding domain comprises CDR1 of SEQ ID NO: 91, CDR2 of SEQ ID NO: 92, and CDR3 of SEQ ID NO: 93. In yet another embodiment, the albumin binding domain comprises an amino acid sequence of SEQ ID NO: 90 or 97. In yet another embodiment, the albumin binding domain comprises an amino acid sequence of SEQ ID NO: 90. In still another embodiment, the albumin binding domain comprises an amino acid sequence of SEQ ID NO: 97.

In certain embodiments, the antibody is a human antibody. In certain embodiments, the antibody is a humanized antibody.

In certain embodiments, the antibody is a single domain antibody (sdAb) that binds to an albumin. In certain embodiments, the antibody is an sdAb that binds to an HSA.

In certain embodiments, the sdAb binds to an HSA with a K_{d} ranging from about 10 pM to about 1,000 nM, from about 1 to about 500 nM, from about 1 to about 200 nM, or from about 1 to about 100 nM. In certain embodiments, the sdAb binds to an HSA with a K_{d} ranging from about 10 pM to about 1,000 nM. In certain embodiments, the sdAb binds to an HSA with a K_{d} ranging from about 1 to about 500 nM. In certain embodiments, the sdAb binds to an HSA with a K_{d} ranging from about 1 to about 200 nM. In certain embodiments, the sdAb binds to an HSA with a K_{d} ranging from about 1 to about 100 nM.

In one embodiment, the sdAb comprises (i) CDR1 of SEQ ID NO: 83, CDR2 of SEQ ID NO: 84, and CDR3 of SEQ ID NO: 85; or (ii) CDR1 of SEQ ID NO: 91, CDR2 of SEQ ID NO: 92, and CDR3 of SEQ ID NO: 93. In another embodiment, the sdAb comprises CDR1 of SEQ ID NO: 83, CDR2 of SEQ ID NO: 84, and CDR3 of SEQ ID NO: 85. In yet another embodiment, the sdAb comprises CDR1 of SEQ ID NO: 91, CDR2 of SEQ ID NO: 92, and CDR3 of SEQ ID NO: 93.

In one embodiment, the sdAb has the structure of FR1-CDR1-FR2-CDR2-FR3-CDR3-FR4, wherein:
CDR1, CDR2, and CDR3 are:
   (i) CDR1 of SEQ ID NO: 83, CDR2 of SEQ ID NO: 84, and CDR3 of SEQ ID NO: 85; or
   (ii) CDR1 of SEQ ID NO: 91, CDR2 of SEQ ID NO: 92, and CDR3 of SEQ ID NO: 93;
FR1 is an amino acid sequence of SEQ ID NO: 86 or 94;
FR2 is an amino acid sequence of SEQ ID NO: 87 or 95;
FR3 is an amino acid sequence of SEQ ID NO: 88; and
FR4 is an amino acid sequence of SEQ ID NO: 89 or 96.

In another embodiment, the sdAb has the structure of FR1-CDR1-FR2-CDR2-FR3-CDR3-FR4, wherein:
CDR1, CDR2, and CDR3 are:
   (i) CDR1 of SEQ ID NO: 83, CDR2 of SEQ ID NO: 84, and CDR3 of SEQ ID NO: 85; or
   (ii) CDR1 of SEQ ID NO: 91, CDR2 of SEQ ID NO: 92, and CDR3 of SEQ ID NO: 93;
FR1 is an amino acid sequence of SEQ ID NO: 86;
FR2 is an amino acid sequence of SEQ ID NO: 87;
FR3 is an amino acid sequence of SEQ ID NO: 88; and
FR3 is an amino acid sequence of SEQ ID NO: 89.

In yet another embodiment, the sdAb has the structure of FR1-CDR1-FR2-CDR2-FR3-CDR3-FR4, wherein:
CDR1, CDR2, and CDR3 are:
   (i) CDR1 of SEQ ID NO: 83, CDR2 of SEQ ID NO: 84, and CDR3 of SEQ ID NO: 85; or
   (ii) CDR1 of SEQ ID NO: 91, CDR2 of SEQ ID NO: 92, and CDR3 of SEQ ID NO: 93;
FR1 is an amino acid sequence of SEQ ID NO: 94;
FR2 is an amino acid sequence of SEQ ID NO: 95;
FR3 is an amino acid sequence of SEQ ID NO: 88; and
FR3 is an amino acid sequence of SEQ ID NO: 96.

In one embodiment, the sdAb has an amino acid sequence of SEQ ID NO: 90 or 97. In another embodiment, the sdAb has an amino acid sequence of SEQ ID NO: 90. In yet another embodiment, the sdAb has an amino acid sequence of SEQ ID NO: 97.

In certain embodiments, the antibody is a V_{H}H single domain antibody that binds to an albumin. In certain embodiments, the antibody is a V_{H}H single domain antibody that binds to an HSA.

In certain embodiments, the V_{H}H single domain antibody binds to an HSA with a K_{d} ranging from about 10 pM to about 1,000 nM, from about 1 to about 500 nM, from about 1 to about 200 nM, or from about 1 to about 100 nM. In certain embodiments, the V_{H}H single domain antibody binds to an HSA with a K_{d} ranging from about 10 pM to about 1,000 nM. In certain embodiments, the V_{H}H single domain antibody binds to an HSA with a K_{d} ranging from about 1 to about 500 nM. In certain embodiments, the V_{H}H single domain antibody binds to an HSA with a K_{d} ranging from about 1 to about 200 nM. In certain embodiments, the V_{H}H single domain antibody binds to an HSA with a K_{d} ranging from about 1 to about 100 nM.

In one embodiment, the V_{H}H single domain antibody comprises (i) heavy chain CDR1 of SEQ ID NO: 83, heavy chain CDR2 of SEQ ID NO: 84, and heavy chain CDR3 of SEQ ID NO: 85; or (ii) heavy chain CDR1 of SEQ ID NO: 91, heavy chain CDR2 of SEQ ID NO: 92, and heavy chain CDR3 of SEQ ID NO: 93. In another embodiment, the V_{H}H single domain antibody comprises heavy chain CDR1 of SEQ ID NO: 83, heavy chain CDR2 of SEQ ID NO: 84, and heavy chain CDR3 of SEQ ID NO: 85. In yet another embodiment, the V_{H}H single domain antibody comprises heavy chain CDR1 of SEQ ID NO: 91, heavy chain CDR2 of SEQ ID NO: 92, and heavy chain CDR3 of SEQ ID NO: 93.

In one embodiment, the V_{H}H single domain antibody has the structure of FR1-CDR1-FR2-CDR2-FR3-CDR3-FR4, wherein:
CDR1, CDR2, and CDR3 are:
   (i) CDR1 of SEQ ID NO: 83, CDR2 of SEQ ID NO: 84, and CDR3 of SEQ ID NO: 85; or
   (ii) CDR1 of SEQ ID NO: 91, CDR2 of SEQ ID NO: 92, and CDR3 of SEQ ID NO: 93;
FR1 is an amino acid sequence of SEQ ID NO: 86 or 94;
FR2 is an amino acid sequence of SEQ ID NO: 87 or 95;
FR3 is an amino acid sequence of SEQ ID NO: 88; and
FR4 is an amino acid sequence of SEQ ID NO: 89 or 96.

In another embodiment, the V_{H}H single domain antibody has the structure of FR1-CDR1-FR2-CDR2-FR3-CDR3-FR4, wherein:
CDR1, CDR2, and CDR3 are:
   (i) CDR1 of SEQ ID NO: 83, CDR2 of SEQ ID NO: 84, and CDR3 of SEQ ID NO: 85; or
   (ii) CDR1 of SEQ ID NO: 91, CDR2 of SEQ ID NO: 92, and CDR3 of SEQ ID NO: 93;
FR1 is an amino acid sequence of SEQ ID NO: 86;
FR2 is an amino acid sequence of SEQ ID NO: 87;
FR3 is an amino acid sequence of SEQ ID NO: 88; and
FR3 is an amino acid sequence of SEQ ID NO: 89.

In yet another embodiment, the V_{H}H single domain antibody has the structure of FR1-CDR1-FR2-CDR2-FR3-CDR3-FR4, wherein:
CDR1, CDR2, and CDR3 are:
   (i) CDR1 of SEQ ID NO: 85, CDR2 of SEQ ID NO: 86, and CDR3 of SEQ ID NO: 87; or
   (ii) CDR1 of SEQ ID NO: 93, CDR2 of SEQ ID NO: 94, and CDR3 of SEQ ID NO: 95;
FR1 is an amino acid sequence of SEQ ID NO: 94;
FR2 is an amino acid sequence of SEQ ID NO: 95;
FR3 is an amino acid sequence of SEQ ID NO: 88; and
FR3 is an amino acid sequence of SEQ ID NO: 96.

In one embodiment, the V_{H}H single domain antibody has an amino acid sequence of SEQ ID NO: 90 or 97. In another embodiment, the V_{H}H single domain antibody has an amino acid sequence of SEQ ID NO: 90. In yet another embodiment, the V_{H}H single domain antibody has an amino acid sequence of SEQ ID NO: 97.

In certain embodiments, the V_{H}H single domain antibody is a human antibody. In certain embodiments, the V_{H}H single domain antibody is a humanized antibody.

In one embodiment, provided herein is a fusion protein comprising an IL-2 domain, a PD-1 binding domain, and a half-life-extension domain that comprises a fragment crystallizable (Fc) domain. In certain embodiments, the IL-2 domain causes the fusion protein to signal through a receptor comprising CD122 and CD132 subunits.

In another embodiment, provided herein is a fusion protein comprising an IL-2 domain, a PD-1 binding domain, and a half-life-extension domain that comprises an Fc domain; wherein the IL-2 domain causes the fusion protein to signal through a receptor consisting of CD122 and CD132 subunits.

In yet another embodiment, provided herein is a fusion protein comprising an IL-2 domain, first and second PD-1 binding domains, a half-life-extension domain that comprises an Fc domain having first and second peptide chains, and optionally a peptide linker; wherein the N-terminus of the IL-2 domain is connected to the C-terminus of the first peptide chain of the Fc domain directly or via the peptide linker.

In yet another embodiment, provided herein is a fusion protein comprising an IL-2 domain, first and second PD-1 binding domains, a half-life-extension domain that comprises an Fc domain having first and second peptide chains, and a peptide linker; wherein the N-terminus of the IL-2 domain is connected to the C-terminus of the first peptide chain of the Fc domain via the peptide linker.

In yet another embodiment, provided herein is a fusion protein comprising first and second IL-2 domains, first and second PD-1 binding domains, a half-life-extension domain that comprises an Fc domain having first and second peptide chains, and optionally first and second peptide linkers; wherein the N-terminus of the first IL-2 domain is connected to the C-terminus of the first peptide chain of the Fe domain directly or via the first peptide linker; and the N-terminus of the second IL-2 domain is connected to the C-terminus of the second peptide chain of the Fc domain directly or via the second peptide linker.

In yet another embodiment, provided herein is a fusion protein comprising first and second IL-2 domains, first and second PD-1 binding domains, a half-life-extension domain that comprises an Fc domain having first and second peptide chains, and first and second peptide linkers; wherein the N-terminus of the first IL-2 domain is connected to the C-terminus of the first peptide chain of the Fc domain via the first peptide linker; and the N-terminus of the second IL-2 domain is connected to the C-terminus of the second peptide chain of the Fc domain via the second peptide linker.

In yet another embodiment, provided herein is a fusion protein comprising an IL-2 domain, first and second PD-1 binding domains, a half-life-extension domain that comprises an Fc, and optionally a peptide linker; wherein the C-terminus of the IL-2 domain is connected to an N-terminus of the first PD-1 binding domain directly or via the peptide linker.

In still another embodiment, provided herein is a fusion protein comprising an IL-2 domain, first and second PD-1 binding domains, a half-life-extension domain that comprises an Fc, and a peptide linker; wherein the C-terminus of the IL-2 domain is connected to an N-terminus of the first PD-1 binding domain via the peptide linker.

In one embodiment, the Fc domain is a hIgG1 Fc domain or a mutein thereof, or a fragment thereof. In another embodiment, the Fc domain is a hIgG1 Fc having an amino acid substitution of N297A. In yet another embodiment, the Fc domain is a hIgG2 Fc domain or a mutein thereof, or a fragment thereof. In still another embodiment, the Fc domain is a hIgG4 Fc domain or a mutein thereof, or a fragment thereof.

In one embodiment, the FC domain as a half-life-extension domain comprises an amino acid sequence of SEQ ID NO: 98, 99, 100, 101, 102, 103, 104, 105, 106, 107, 108, or 109. In another embodiment, the FC domain as a half-life-extension domain comprises an amino acid sequence of SEQ ID NO: 98. In yet another embodiment, the FC domain as a half-life-extension domain comprises an amino acid sequence of SEQ ID NO: 99. In yet another embodiment, the FC domain as a half-life-extension domain comprises an amino acid sequence of SEQ ID NO: 100. In yet another embodiment, the FC domain as a half-life-extension domain comprises an amino acid sequence of SEQ ID NO: 101. In yet another embodiment, the FC domain as a half-life-extension domain comprises an amino acid sequence of SEQ ID NO: 102. In yet another embodiment, the FC domain as a half-life-extension domain comprises an amino acid sequence of SEQ ID NO: 103. In yet another embodiment, the FC domain as a half-life-extension domain comprises an amino acid sequence of SEQ ID NO: 104. In yet another embodiment, the FC domain as a half-life-extension domain comprises an amino acid sequence of SEQ ID NO: 105. In yet another embodiment, the FC domain as a half-life-extension domain comprises an amino acid sequence of SEQ ID NO: 106. In yet another embodiment, the FC domain as a half-life-extension domain comprises an amino acid sequence of SEQ ID NO: 107. In yet another embodiment, the FC domain as a half-life-extension domain comprises an amino acid sequence of SEQ ID NO: 108. In still another embodiment, the FC domain as a half-life-extension domain comprises an amino acid sequence of SEQ ID NO: 109.

In one embodiment, the FC domain as a half-life-extension domain comprises a pair of chains in a knobs-in-holes (KIH) configuration. Thus, in one embodiment, the FC domain as a half-life-extension domain comprises an amino acid sequence pair of SEQ ID NOs: 101 and 102, 103 and 104, 105 and 106, or 108 and 109 in a knobs-in-holes configuration. In another embodiment, the FC domain as a half-life-extension domain comprises an amino acid sequence pair of SEQ ID NOs: 101 and 102 in a knobs-in-holes configuration. In yet another embodiment, the FC domain as a half-life-extension domain comprises an amino acid sequence pair of SEQ ID NOs: 103 and 104 in a knobs-in-holes configuration. In yet another embodiment, the Fc domain as a half-life-extension domain comprises an amino acid sequence pair of SEQ ID NOs: 105 and 106 in a knobs-in-holes configuration. In still another embodiment, the FC domain as a half-life-extension domain comprises an amino acid sequence pair of SEQ ID NOs: 108 and 109 in a knobs-in-holes configuration.

In one embodiment, the PD-1 binding domain and the half-life-extension domain that comprises an Fc are a part of an intact anti-PD-1 antibody comprising two light chains and two heavy chains.

Thus, in one embodiment, provided herein is a fusion protein comprising an intact anti-PD-1 antibody comprising two light chains and two heavy chains, an IL-2 domain, and optionally a peptide linker.

In another embodiment, provided herein is a fusion protein comprising an IL-2 domain, an intact anti-PD-1 antibody comprising two light chains and first and second heavy chains, and optionally a peptide linker, wherein the N-terminus of the IL-2 domain is connected to the C-terminus of the first heavy chain directly or via the peptide linker.

In yet another embodiment, provided herein is a fusion protein comprising an IL-2 domain, an intact anti-PD-1 antibody comprising two light chains and first and second heavy chains, and a peptide linker, wherein the N-terminus of the IL-2 domain is connected to the C-terminus of the first heavy chain via the peptide linker.

In yet another embodiment, provided herein is a fusion protein comprising first and second IL-2 domains, an anti-PD-1 antibody comprising two light chains and first and second heavy chains, and optionally first and second peptide linkers, wherein the N-terminus of the first IL-2 domain is connected to the C-terminus of the first heavy chain directly or via the first peptide linker; and the N-terminus of the second IL-2 domain is connected to the C-terminus of the second heavy chain directly or via the second peptide linker.

In yet another embodiment, provided herein is a fusion protein comprising first and second IL-2 domains, an anti-PD-1 antibody comprising light chains and first and second heavy chains, and first and second peptide linkers, wherein the N-terminus of the first IL-2 domain is connected to the C-terminus of the first heavy chain via the first peptide linker; and the N-terminus of the second IL-2 domain is connected to the C-terminus of the second heavy chain via the second peptide linker.

In yet another embodiment, provided herein is a fusion protein comprising an IL-2 domain, an intact anti-PD-1 antibody comprising two light chains and first and second heavy chains, and optionally a peptide linker, wherein the C-terminus of the IL-2 domain is connected to the N-terminus of the first heavy chain directly or via the peptide linker.

In still another embodiment, provided herein is a fusion protein comprising an IL-2 domain, an intact anti-PD-1 antibody comprising first and second light chains and two heavy chains, and optionally a peptide linker, wherein the C-terminus of the IL-2 domain is connected to the N-terminus of the first light chain directly or via the peptide linker.

In one embodiment, provided herein is a fusion protein comprising an IL-2 domain of any one of SEQ ID NOs: 42 to 81, 255, and 256, an intact anti-PD-1 antibody comprising two light chains and first and second heavy chains, and a peptide linker (e.g., of SEQ ID NO: 215, wherein the N-terminus of the IL-2 domain is connected to the C-terminus of the first heavy chain via the peptide linker; and wherein the anti-PD-1 antibody comprises a CDRL1 of SEQ ID NO: 111, a CDRL2 of DAS, a CDRL3 of SEQ ID NO: 112, a CDRH1 of SEQ ID NO: 113, a CDRH2 of SEQ ID NO: 114, and a CDRH3 of SEQ ID NO: 115.

In another embodiment, provided herein is a fusion protein comprising an IL-2 domain of SEQ ID NO: 56, 60, 68, 70, 80, 255, or 256, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein the anti-PD-1 antibody comprises a CDRL1 of SEQ ID NO: 111, a CDRL2 of DAS, a CDRL3 of SEQ ID NO: 112, a CDRH1 of SEQ ID NO: 113, a CDRH2 of SEQ ID NO: 114, and a CDRH3 of SEQ ID NO: 115.

In yet another embodiment, provided herein is a fusion protein comprising an IL-2 domain of SEQ ID NO: 42, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein the anti-PD-1 antibody comprises a CDRL1 of SEQ ID NO: 111, a CDRL2 of DAS, a CDRL3 of SEQ ID NO: 112, a CDRH1 of SEQ ID NO: 113, a CDRH2 of SEQ ID NO: 114, and a CDRH3 of SEQ ID NO: 115.

In yet another embodiment, provided herein is a fusion protein comprising an IL-2 domain of SEQ ID NO: 50, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein the anti-PD-1 antibody comprises a CDRL1 of SEQ ID NO: 111, a CDRL2 of DAS, a CDRL3 of SEQ ID NO: 112, a CDRH1 of SEQ ID NO: 113, a CDRH2 of SEQ ID NO: 114, and a CDRH3 of SEQ ID NO: 115.

In yet another embodiment, provided herein is a fusion protein comprising an IL-2 domain of SEQ ID NO: 52, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein the anti-PD-1 antibody comprises a CDRL1 of SEQ ID NO: 111, a CDRL2 of DAS, a CDRL3 of SEQ ID NO: 112, a CDRH1 of SEQ ID NO: 113, a CDRH2 of SEQ ID NO: 114, and a CDRH3 of SEQ ID NO: 115.

In yet another embodiment, provided herein is a fusion protein comprising an IL-2 domain of SEQ ID NO: 54, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein the anti-PD-1 antibody comprises a CDRL1 of SEQ ID NO: 111, a CDRL2 of DAS, a CDRL3 of SEQ ID NO: 112, a CDRH1 of SEQ ID NO: 113, a CDRH2 of SEQ ID NO: 114, and a CDRH3 of SEQ ID NO: 115.

In yet another embodiment, provided herein is a fusion protein comprising an IL-2 domain of SEQ ID NO: 56, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein the anti-PD-1 antibody comprises a CDRL1 of SEQ ID NO: 111, a CDRL2 of DAS, a CDRL3 of SEQ ID NO: 112, a CDRH1 of SEQ ID NO: 113, a CDRH2 of SEQ ID NO: 114, and a CDRH3 of SEQ ID NO: 115.

In yet another embodiment, provided herein is a fusion protein comprising an IL-2 domain of SEQ ID NO: 60, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein the anti-PD-1 antibody comprises a CDRL1 of SEQ ID NO: 111, a CDRL2 of DAS, a CDRL3 of SEQ ID NO: 112, a CDRH1 of SEQ ID NO: 113, a CDRH2 of SEQ ID NO: 114, and a CDRH3 of SEQ ID NO: 115.

In yet another embodiment, provided herein is a fusion protein comprising an IL-2 domain of SEQ ID NO: 68, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein the anti-PD-1 antibody comprises a CDRL1 of SEQ ID NO: 111, a CDRL2 of DAS, a CDRL3 of SEQ ID NO: 112, a CDRH1 of SEQ ID NO: 113, a CDRH2 of SEQ ID NO: 114, and a CDRH3 of SEQ ID NO: 115.

In yet another embodiment, provided herein is a fusion protein comprising an IL-2 domain of SEQ ID NO: 70, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein the anti-PD-1 antibody comprises a CDRL1 of SEQ ID NO: 111, a CDRL2 of DAS, a CDRL3 of SEQ ID NO: 112, a CDRH1 of SEQ ID NO: 113, a CDRH2 of SEQ ID NO: 114, and a CDRH3 of SEQ ID NO: 115.

In yet another embodiment, provided herein is a fusion protein comprising an IL-2 domain of SEQ ID NO: 80, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein the anti-PD-1 antibody comprises a CDRL1 of SEQ ID NO: 111, a CDRL2 of DAS, a CDRL3 of SEQ ID NO: 112, a CDRH1 of SEQ ID NO: 113, a CDRH2 of SEQ ID NO: 114, and a CDRH3 of SEQ ID NO: 115.

In still another embodiment, provided herein is a fusion protein comprising an IL-2 domain of SEQ ID NO: 256, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein the anti-PD-1 antibody comprises a CDRL1 of SEQ ID NO: 111, a CDRL2 of DAS, a CDRL3 of SEQ ID NO: 112, a CDRH1 of SEQ ID NO: 113, a CDRH2 of SEQ ID NO: 114, and a CDRH3 of SEQ ID NO: 115.

In one embodiment, provided herein is a fusion protein comprising an IL-2 domain of any one of SEQ ID NOs: 42 to 81, 255, and 256, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein each light chain comprises the amino acid sequence of SEQ ID NO: 116 and each heavy is of SEQ ID NO: 117.

In another embodiment, provided herein is a fusion protein comprising an IL-2 domain of SEQ ID NO: 56, 60, 68, 70, 80, 255, or 256, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein each light chain comprises the amino acid sequence of SEQ ID NO: 116 and each heavy chain comprises the amino acid sequence of SEQ ID NO: 117.

In yet another embodiment, provided herein is a fusion protein comprising an IL-2 domain of SEQ ID NO: 42, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein each light chain comprises the amino acid sequence of SEQ ID NO: 116 and each heavy chain comprises the amino acid sequence of SEQ ID NO: 117.

In yet another embodiment, provided herein is a fusion protein comprising an IL-2 domain of SEQ ID NO: 50, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein each light chain comprises the amino acid sequence of SEQ ID NO: 116 and each heavy chain comprises the amino acid sequence of SEQ ID NO: 117.

In yet another embodiment, provided herein is a fusion protein comprising an IL-2 domain of SEQ ID NO: 52, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein each light chain comprises the amino acid sequence of SEQ ID NO: 116 and each heavy chain comprises the amino acid sequence of SEQ ID NO: 117.

In yet another embodiment, provided herein is a fusion protein comprising an IL-2 domain of SEQ ID NO: 54, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein each light chain comprises the amino acid sequence of SEQ ID NO: 116 and each heavy chain comprises the amino acid sequence of SEQ ID NO: 117.

In yet another embodiment, provided herein is a fusion protein comprising an IL-2 domain of SEQ ID NO: 56, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein each light chain comprises the amino acid sequence of SEQ ID NO: 116 and each heavy chain comprises the amino acid sequence of SEQ ID NO: 117.

In yet another embodiment, provided herein is a fusion protein comprising an IL-2 domain of SEQ ID NO: 60, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein each light chain comprises the amino acid sequence of SEQ ID NO: 116 and each heavy chain comprises the amino acid sequence of SEQ ID NO: 117.

In yet another embodiment, provided herein is a fusion protein comprising an IL-2 domain of SEQ ID NO: 68, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein each light chain comprises the amino acid sequence of SEQ ID NO: 116 and each heavy chain comprises the amino acid sequence of SEQ ID NO: 117.

In yet another embodiment, provided herein is a fusion protein comprising an IL-2 domain of SEQ ID NO: 70, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein each light chain comprises the amino acid sequence of SEQ ID NO: 116 and each heavy chain comprises the amino acid sequence of SEQ ID NO: 117.

In yet another embodiment, provided herein is a fusion protein comprising an IL-2 domain of SEQ ID NO: 80, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein each light chain comprises the amino acid sequence of SEQ ID NO: 116 and each heavy chain comprises the amino acid sequence of SEQ ID NO: 117.

In still another embodiment, provided herein is a fusion protein comprising an IL-2 domain of SEQ ID NO: 256, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein each light chain comprises the amino acid sequence of SEQ ID NO: 116 and each heavy chain comprises the amino acid sequence of SEQ ID NO: 117.

In one embodiment, provided herein is a fusion protein comprising first and second IL-2 domains, each independently of any one of SEQ ID NOs: 42 to 81, 255, and 256; an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein the anti-PD-1 antibody comprises a CDRL1 of SEQ ID NO: 111, a CDRL2 of DAS, a CDRL3 of SEQ ID NO: 112, a CDRH1 of SEQ ID NO: 113, a CDRH2 of SEQ ID NO: 114, and a CDRH3 of SEQ ID NO: 115.

In another embodiment, provided herein is a fusion protein comprising first and second IL-2 domains, each independently of SEQ ID NO: 56, 60, 68, 70, 80, 255, or 256; an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein the anti-PD-1 antibody comprises a CDRL1 of SEQ ID NO: 111, a CDRL2 of DAS, a CDRL3 of SEQ ID NO: 112, a CDRH1 of SEQ ID NO: 113, a CDRH2 of SEQ ID NO: 114, and a CDRH3 of SEQ ID NO: 115.

In yet another embodiment, provided herein is a fusion protein comprising first and second IL-2 domains, each of SEQ ID NO: 42; an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein the anti-PD-1 antibody comprises a CDRL1 of SEQ ID NO: 111, a CDRL2 of DAS, a CDRL3 of SEQ ID NO: 112, a CDRH1 of SEQ ID NO: 113, a CDRH2 of SEQ ID NO: 114, and a CDRH3 of SEQ ID NO: 115.

In yet another embodiment, provided herein is a fusion protein comprising first and second IL-2 domains, each of SEQ ID NO: 50; an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein the anti-PD-1 antibody comprises a CDRL1 of SEQ ID NO: 111, a CDRL2 of DAS, a CDRL3 of SEQ ID NO: 112, a CDRH1 of SEQ ID NO: 113, a CDRH2 of SEQ ID NO: 114, and a CDRH3 of SEQ ID NO: 115.

In yet another embodiment, provided herein is a fusion protein comprising first and second IL-2 domains, each of SEQ ID NO: 52; an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein the anti-PD-1 antibody comprises a CDRL1 of SEQ ID NO: 111, a CDRL2 of DAS, a CDRL3 of SEQ ID NO: 112, a CDRH1 of SEQ ID NO: 113, a CDRH2 of SEQ ID NO: 114, and a CDRH3 of SEQ ID NO: 115.

In yet another embodiment, provided herein is a fusion protein comprising first and second IL-2 domains, each of SEQ ID NO: 54; an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein the anti-PD-1 antibody comprises a CDRL1 of SEQ ID NO: 111, a CDRL2 of DAS, a CDRL3 of SEQ ID NO: 112, a CDRH1 of SEQ ID NO: 113, a CDRH2 of SEQ ID NO: 114, and a CDRH3 of SEQ ID NO: 115.

In yet another embodiment, provided herein is a fusion protein comprising first and second IL-2 domains, each of SEQ ID NO: 56; an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein the anti-PD-1 antibody comprises a CDRL1 of SEQ ID NO: 111, a CDRL2 of DAS, a CDRL3 of SEQ ID NO: 112, a CDRH1 of SEQ ID NO: 113, a CDRH2 of SEQ ID NO: 114, and a CDRH3 of SEQ ID NO: 115.

In yet another embodiment, provided herein is a fusion protein comprising first and second IL-2 domains, each of SEQ ID NO: 60; an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein the anti-PD-1 antibody comprises a CDRL1 of SEQ ID NO: 111, a CDRL2 of DAS, a CDRL3 of SEQ ID NO: 112, a CDRH1 of SEQ ID NO: 113, a CDRH2 of SEQ ID NO: 114, and a CDRH3 of SEQ ID NO: 115.

In yet another embodiment, provided herein is a fusion protein comprising first and second IL-2 domains, each of SEQ ID NO: 68; an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein the anti-PD-1 antibody comprises a CDRL1 of SEQ ID NO: 111, a CDRL2 of DAS, a CDRL3 of SEQ ID NO: 112, a CDRH1 of SEQ ID NO: 113, a CDRH2 of SEQ ID NO: 114, and a CDRH3 of SEQ ID NO: 115.

In yet another embodiment, provided herein is a fusion protein comprising first and second IL-2 domains, each of SEQ ID NO: 70; an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein the anti-PD-1 antibody comprises a CDRL1 of SEQ ID NO: 111, a CDRL2 of DAS, a CDRL3 of SEQ ID NO: 112, a CDRH1 of SEQ ID NO: 113, a CDRH2 of SEQ ID NO: 114, and a CDRH3 of SEQ ID NO: 115.

In yet another embodiment, provided herein is a fusion protein comprising first and second IL-2 domains, each of SEQ ID NO: 80; an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein the anti-PD-1 antibody comprises a CDRL1 of SEQ ID NO: 111, a CDRL2 of DAS, a CDRL3 of SEQ ID NO: 112, a CDRH1 of SEQ ID NO: 113, a CDRH2 of SEQ ID NO: 114, and a CDRH3 of SEQ ID NO: 115.

In still another embodiment, provided herein is a fusion protein comprising first and second IL-2 domains, each of SEQ ID NO: 256; an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein the anti-PD-1 antibody comprises a CDRL1 of SEQ ID NO: 111, a CDRL2 of DAS, a CDRL3 of SEQ ID NO: 112, a CDRH1 of SEQ ID NO: 113, a CDRH2 of SEQ ID NO: 114, and a CDRH3 of SEQ ID NO: 115.

In one embodiment, provided herein is a fusion protein comprising first and second IL-2 domains, each independently of any one of SEQ ID NOs: 42 to 81, 255, and 256, an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein each light chain comprises the amino acid sequence of SEQ ID NO: 116 and each heavy chain comprises the amino acid sequence of SEQ ID NO: 117.

In another embodiment, provided herein is a fusion protein comprising first and second IL-2 domains, each independently of SEQ ID NO: 56, 60, 68, 70, 80, 255, or 256, an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein each light chain comprises the amino acid sequence of SEQ ID NO: 116 and each heavy chain comprises the amino acid sequence of SEQ ID NO: 117.

In yet another embodiment, provided herein is a fusion protein comprising first and second IL-2 domains, each of SEQ ID NO: 42, an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein each light chain comprises the amino acid sequence of SEQ ID NO: 116 and each heavy chain comprises the amino acid sequence of SEQ ID NO: 117.

In yet another embodiment, provided herein is a fusion protein comprising first and second IL-2 domains, each of SEQ ID NO: 50, an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein each light chain comprises the amino acid sequence of SEQ ID NO: 116 and each heavy chain comprises the amino acid sequence of SEQ ID NO: 117.

In yet another embodiment, provided herein is a fusion protein comprising first and second IL-2 domains, each of SEQ ID NO: 52, an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein each light chain comprises the amino acid sequence of SEQ ID NO: 116 and each heavy chain comprises the amino acid sequence of SEQ ID NO: 117.

In yet another embodiment, provided herein is a fusion protein comprising first and second IL-2 domains, each of SEQ ID NO: 54, an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein each light chain comprises the amino acid sequence of SEQ ID NO: 116 and each heavy chain comprises the amino acid sequence of SEQ ID NO: 117.

In yet another embodiment, provided herein is a fusion protein comprising first and second IL-2 domains, each of SEQ ID NO: 56, an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein each light chain comprises the amino acid sequence of SEQ ID NO: 116 and each heavy chain comprises the amino acid sequence of SEQ ID NO: 117.

In yet another embodiment, provided herein is a fusion protein comprising first and second IL-2 domains, each of SEQ ID NO: 60, an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein each light chain comprises the amino acid sequence of SEQ ID NO: 116 and each heavy chain comprises the amino acid sequence of SEQ ID NO: 117.

In yet another embodiment, provided herein is a fusion protein comprising first and second IL-2 domains, each of SEQ ID NO: 68, an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein each light chain comprises the amino acid sequence of SEQ ID NO: 116 and each heavy chain comprises the amino acid sequence of SEQ ID NO: 117.

In yet another embodiment, provided herein is a fusion protein comprising first and second IL-2 domains, each of SEQ ID NO: 70, an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein each light chain comprises the amino acid sequence of SEQ ID NO: 116 and each heavy chain comprises the amino acid sequence of SEQ ID NO: 117.

In yet another embodiment, provided herein is a fusion protein comprising first and second IL-2 domains, each of SEQ ID NO: 80, an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein each light chain comprises the amino acid sequence of SEQ ID NO: 116 and each heavy chain comprises the amino acid sequence of SEQ ID NO: 117.

In still another embodiment, provided herein is a fusion protein comprising first and second IL-2 domains, each of SEQ ID NO: 256, an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein each light chain comprises the amino acid sequence of SEQ ID NO: 116 and each heavy chain comprises the amino acid sequence of SEQ ID NO: 117.

In one embodiment, provided herein is a fusion protein comprising an IL-2 domain of any one of SEQ ID NOs: 42 to 81, 255, and 256, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein the anti-PD-1 antibody comprises a CDRL1 of SEQ ID NO: 129, a CDRL2 of KVS, a CDRL1 of SEQ ID NO: 130, a CDRH1 of SEQ ID NO: 131, a CDRH2 of SEQ ID NO: 132, and a CDRH3 of SEQ ID NO: 133.

In another embodiment, provided herein is a fusion protein comprising an IL-2 domain of SEQ ID NO: 56, 60, 68, 70, 80, 255, or 256, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein the anti-PD-1 antibody comprises a CDRL1 of SEQ ID NO: 129, a CDRL2 of KVS, a CDRL1 of SEQ ID NO: 130, a CDRH1 of SEQ ID NO: 131, a CDRH2 of SEQ ID NO: 132, and a CDRH3 of SEQ ID NO: 133.

In yet another embodiment, provided herein is a fusion protein comprising an IL-2 domain of SEQ ID NO: 42, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein the anti-PD-1 antibody comprises a CDRL1 of SEQ ID NO: 129, a CDRL2 of KVS, a CDRL1 of SEQ ID NO: 130, a CDRH1 of SEQ ID NO: 131, a CDRH2 of SEQ ID NO: 132, and a CDRH3 of SEQ ID NO: 133.

In yet another embodiment, provided herein is a fusion protein comprising an IL-2 domain of SEQ ID NO: 50, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein the anti-PD-1 antibody comprises a CDRL1 of SEQ ID NO: 129, a CDRL2 of KVS, a CDRL1 of SEQ ID NO: 130, a CDRH1 of SEQ ID NO: 131, a CDRH2 of SEQ ID NO: 132, and a CDRH3 of SEQ ID NO: 133.

In yet another embodiment, provided herein is a fusion protein comprising an IL-2 domain of SEQ ID NO: 52, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein the anti-PD-1 antibody comprises a CDRL1 of SEQ ID NO: 129, a CDRL2 of KVS, a CDRL1 of SEQ ID NO: 130, a CDRH1 of SEQ ID NO: 131, a CDRH2 of SEQ ID NO: 132, and a CDRH3 of SEQ ID NO: 133.

In yet another embodiment, provided herein is a fusion protein comprising an IL-2 domain of SEQ ID NO: 54, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein the anti-PD-1 antibody comprises a CDRL1 of SEQ ID NO: 129, a CDRL2 of KVS, a CDRL1 of SEQ ID NO: 130, a CDRH1 of SEQ ID NO: 131, a CDRH2 of SEQ ID NO: 132, and a CDRH3 of SEQ ID NO: 133.

In yet another embodiment, provided herein is a fusion protein comprising an IL-2 domain of SEQ ID NO: 56, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein the anti-PD-1 antibody comprises a CDRL1 of SEQ ID NO: 129, a CDRL2 of KVS, a CDRL1 of SEQ ID NO: 130, a CDRH1 of SEQ ID NO: 131, a CDRH2 of SEQ ID NO: 132, and a CDRH3 of SEQ ID NO: 133.

In yet another embodiment, provided herein is a fusion protein comprising an IL-2 domain of SEQ ID NO: 60, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein the anti-PD-1 antibody comprises a CDRL1 of SEQ ID NO: 129, a CDRL2 of KVS, a CDRL1 of SEQ ID NO: 130, a CDRH1 of SEQ ID NO: 131, a CDRH2 of SEQ ID NO: 132, and a CDRH3 of SEQ ID NO: 133.

In yet another embodiment, provided herein is a fusion protein comprising an IL-2 domain of SEQ ID NO: 68, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein the anti-PD-1 antibody comprises a CDRL1 of SEQ ID NO: 129, a CDRL2 of KVS, a CDRL1 of SEQ ID NO: 130, a CDRH1 of SEQ ID NO: 131, a CDRH2 of SEQ ID NO: 132, and a CDRH3 of SEQ ID NO: 133.

In yet another embodiment, provided herein is a fusion protein comprising an IL-2 domain of SEQ ID NO: 70, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein the anti-PD-1 antibody comprises a CDRL1 of SEQ ID NO: 129, a CDRL2 of KVS, a CDRL1 of SEQ ID NO: 130, a CDRH1 of SEQ ID NO: 131, a CDRH2 of SEQ ID NO: 132, and a CDRH3 of SEQ ID NO: 133.

In yet another embodiment, provided herein is a fusion protein comprising an IL-2 domain of SEQ ID NO: 80, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein the anti-PD-1 antibody comprises a CDRL1 of SEQ ID NO: 129, a CDRL2 of KVS, a CDRL1 of SEQ ID NO: 130, a CDRH1 of SEQ ID NO: 131, a CDRH2 of SEQ ID NO: 132, and a CDRH3 of SEQ ID NO: 133.

In still another embodiment, provided herein is a fusion protein comprising an IL-2 domain of SEQ ID NO: 256, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein the anti-PD-1 antibody comprises a CDRL1 of SEQ ID NO: 129, a CDRL2 of KVS, a CDRL1 of SEQ ID NO: 130, a CDRH1 of SEQ ID NO: 131, a CDRH2 of SEQ ID NO: 132, and a CDRH3 of SEQ ID NO: 133.

In one embodiment, provided herein is a fusion protein comprising an IL-2 domain of any one of SEQ ID NOs: 42 to 81, 255, and 256, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein each light chain comprises the amino acid sequence of SEQ ID NO: 134 and each heavy chain comprises the amino acid sequence of SEQ ID NO: 135.

In another embodiment, provided herein is a fusion protein comprising an IL-2 domain of SEQ ID NO: 56, 60, 68, 70, 80, 255, or 256, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein each light chain comprises the amino acid sequence of SEQ ID NO: 134 and each heavy chain comprises the amino acid sequence of SEQ ID NO: 135.

In yet another embodiment, provided herein is a fusion protein comprising an IL-2 domain of SEQ ID NO: 42, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein each light chain comprises the amino acid sequence of SEQ ID NO: 134 and each heavy chain comprises the amino acid sequence of SEQ ID NO: 135.

In yet another embodiment, provided herein is a fusion protein comprising an IL-2 domain of SEQ ID NO: 50, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein each light chain comprises the amino acid sequence of SEQ ID NO: 134 and each heavy chain comprises the amino acid sequence of SEQ ID NO: 135.

In yet another embodiment, provided herein is a fusion protein comprising an IL-2 domain of SEQ ID NO: 52, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein each light chain comprises the amino acid sequence of SEQ ID NO: 134 and each heavy chain comprises the amino acid sequence of SEQ ID NO: 135.

In yet another embodiment, provided herein is a fusion protein comprising an IL-2 domain of SEQ ID NO: 54, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein each light chain comprises the amino acid sequence of SEQ ID NO: 134 and each heavy chain comprises the amino acid sequence of SEQ ID NO: 135.

In yet another embodiment, provided herein is a fusion protein comprising an IL-2 domain of SEQ ID NO: 56, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein each light chain comprises the amino acid sequence of SEQ ID NO: 134 and each heavy chain comprises the amino acid sequence of SEQ ID NO: 135.

In yet another embodiment, provided herein is a fusion protein comprising an IL-2 domain of SEQ ID NO: 60, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein each light chain comprises the amino acid sequence of SEQ ID NO: 134 and each heavy chain comprises the amino acid sequence of SEQ ID NO: 135.

In yet another embodiment, provided herein is a fusion protein comprising an IL-2 domain of SEQ ID NO: 68, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein each light chain comprises the amino acid sequence of SEQ ID NO: 134 and each heavy chain comprises the amino acid sequence of SEQ ID NO: 135.

In yet another embodiment, provided herein is a fusion protein comprising an IL-2 domain of SEQ ID NO: 70, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein each light chain comprises the amino acid sequence of SEQ ID NO: 134 and each heavy chain comprises the amino acid sequence of SEQ ID NO: 135.

In yet another embodiment, provided herein is a fusion protein comprising an IL-2 domain of SEQ ID NO: 80, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein each light chain comprises the amino acid sequence of SEQ ID NO: 134 and each heavy chain comprises the amino acid sequence of SEQ ID NO: 135.

In still another embodiment, provided herein is a fusion protein comprising an IL-2 domain of SEQ ID NO: 256, an intact anti-PD-1 antibody comprising two light chains and first and second heavy chains, and a peptide linker (e.g., of SEQ ID NO: 215), wherein the N-terminus of the IL-2 domain is connected to the C-terminus of the first heavy chain via the peptide linker; and wherein each light chain comprises the amino acid sequence of SEQ ID NO: 134 and each heavy chain comprises the amino acid sequence of SEQ ID NO: 135.

In one embodiment, provided herein is a fusion protein comprising first and second IL-2 domains, each independently of any one of SEQ ID NOs: 42 to 81, 255, and 256; an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein the anti-PD-1 antibody comprises a CDRL1 of SEQ ID NO: 129, a CDRL2 of KVS, a CDRL1 of SEQ ID NO: 130, a CDRH1 of SEQ ID NO: 131, a CDRH2 of SEQ ID NO: 132, and a CDRH3 of SEQ ID NO: 133.

In another embodiment, provided herein is a fusion protein comprising first and second IL-2 domains, each independently of SEQ ID NO: 56, 60, 68, 70, 80, 255, or 256; an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein the anti-PD-1 antibody comprises a CDRL1 of SEQ ID NO: 129, a CDRL2 of KVS, a CDRL1 of SEQ ID NO: 130, a CDRH1 of SEQ ID NO: 131, a CDRH2 of SEQ ID NO: 132, and a CDRH3 of SEQ ID NO: 133.

In yet another embodiment, provided herein is a fusion protein comprising first and second IL-2 domains, each of SEQ ID NO: 42; an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein the anti-PD-1 antibody comprises a CDRL1 of SEQ ID NO: 129, a CDRL2 of KVS, a CDRL1 of SEQ ID NO: 130, a CDRH1 of SEQ ID NO: 131, a CDRH2 of SEQ ID NO: 132, and a CDRH3 of SEQ ID NO: 133.

In yet another embodiment, provided herein is a fusion protein comprising first and second IL-2 domains, each of SEQ ID NO: 50; an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein the anti-PD-1 antibody comprises a CDRL1 of SEQ ID NO: 129, a CDRL2 of KVS, a CDRL1 of SEQ ID NO: 130, a CDRH1 of SEQ ID NO: 131, a CDRH2 of SEQ ID NO: 132, and a CDRH3 of SEQ ID NO: 133.

In yet another embodiment, provided herein is a fusion protein comprising first and second IL-2 domains, each of SEQ ID NO: 52; an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein the anti-PD-1 antibody comprises a CDRL1 of SEQ ID NO: 129, a CDRL2 of KVS, a CDRL1 of SEQ ID NO: 130, a CDRH1 of SEQ ID NO: 131, a CDRH2 of SEQ ID NO: 132, and a CDRH3 of SEQ ID NO: 133.

In yet another embodiment, provided herein is a fusion protein comprising first and second IL-2 domains, each of SEQ ID NO: 54; an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein the anti-PD-1 antibody comprises a CDRL1 of SEQ ID NO: 129, a CDRL2 of KVS, a CDRL1 of SEQ ID NO: 130, a CDRH1 of SEQ ID NO: 131, a CDRH2 of SEQ ID NO: 132, and a CDRH3 of SEQ ID NO: 133.

In yet another embodiment, provided herein is a fusion protein comprising first and second IL-2 domains, each of SEQ ID NO: 56; an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein the anti-PD-1 antibody comprises a CDRL1 of SEQ ID NO: 129, a CDRL2 of KVS, a CDRL1 of SEQ ID NO: 130, a CDRH1 of SEQ ID NO: 131, a CDRH2 of SEQ ID NO: 132, and a CDRH3 of SEQ ID NO: 133.

In yet another embodiment, provided herein is a fusion protein comprising first and second IL-2 domains, each of SEQ ID NO: 60; an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein the anti-PD-1 antibody comprises a CDRL1 of SEQ ID NO: 129, a CDRL2 of KVS, a CDRL1 of SEQ ID NO: 130, a CDRH1 of SEQ ID NO: 131, a CDRH2 of SEQ ID NO: 132, and a CDRH3 of SEQ ID NO: 133.

In yet another embodiment, provided herein is a fusion protein comprising first and second IL-2 domains, each of SEQ ID NO: 68; an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein the anti-PD-1 antibody comprises a CDRL1 of SEQ ID NO: 129, a CDRL2 of KVS, a CDRL1 of SEQ ID NO: 130, a CDRH1 of SEQ ID NO: 131, a CDRH2 of SEQ ID NO: 132, and a CDRH3 of SEQ ID NO: 133.

In yet another embodiment, provided herein is a fusion protein comprising first and second IL-2 domains, each of SEQ ID NO: 70; an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein the anti-PD-1 antibody comprises a CDRL1 of SEQ ID NO: 129, a CDRL2 of KVS, a CDRL1 of SEQ ID NO: 130, a CDRH1 of SEQ ID NO: 131, a CDRH2 of SEQ ID NO: 132, and a CDRH3 of SEQ ID NO: 133.

In yet another embodiment, provided herein is a fusion protein comprising first and second IL-2 domains, each of SEQ ID NO: 80; an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein the anti-PD-1 antibody comprises a CDRL1 of SEQ ID NO: 129, a CDRL2 of KVS, a CDRL1 of SEQ ID NO: 130, a CDRH1 of SEQ ID NO: 131, a CDRH2 of SEQ ID NO: 132, and a CDRH3 of SEQ ID NO: 133.

In still another embodiment, provided herein is a fusion protein comprising first and second IL-2 domains, each of SEQ ID NO: 256; an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein the anti-PD-1 antibody comprises a CDRL1 of SEQ ID NO: 129, a CDRL2 of KVS, a CDRL1 of SEQ ID NO: 130, a CDRH1 of SEQ ID NO: 131, a CDRH2 of SEQ ID NO: 132, and a CDRH3 of SEQ ID NO: 133.

In one embodiment, provided herein is a fusion protein comprising first and second IL-2 domains, each independently of any one of SEQ ID NOs: 42 to 81, 255, and 256, an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein each light chain comprises the amino acid sequence of SEQ ID NO: 134 and each heavy chain comprises the amino acid sequence of SEQ ID NO: 135.

In another embodiment, provided herein is a fusion protein comprising first and second IL-2 domains, each independently of SEQ ID NO: 56, 60, 68, 70, 80, 255, or 256, an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein each light chain comprises the amino acid sequence of SEQ ID NO: 134 and each heavy chain comprises the amino acid sequence of SEQ ID NO: 135.

In yet another embodiment, provided herein is a fusion protein comprising first and second IL-2 domains, each of SEQ ID NO: 42, an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein each light chain comprises the amino acid sequence of SEQ ID NO: 134 and each heavy chain comprises the amino acid sequence of SEQ ID NO: 135.

In yet another embodiment, provided herein is a fusion protein comprising first and second IL-2 domains, each of SEQ ID NO: 50, an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein each light chain comprises the amino acid sequence of SEQ ID NO: 134 and each heavy chain comprises the amino acid sequence of SEQ ID NO: 135.

In yet another embodiment, provided herein is a fusion protein comprising first and second IL-2 domains, each of SEQ ID NO: 52, an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein each light chain comprises the amino acid sequence of SEQ ID NO: 134 and each heavy chain comprises the amino acid sequence of SEQ ID NO: 135.

In yet another embodiment, provided herein is a fusion protein comprising first and second IL-2 domains, each of SEQ ID NO: 54, an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein each light chain comprises the amino acid sequence of SEQ ID NO: 134 and each heavy chain comprises the amino acid sequence of SEQ ID NO: 135.

In yet another embodiment, provided herein is a fusion protein comprising first and second IL-2 domains, each of SEQ ID NO: 56, an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein each light chain comprises the amino acid sequence of SEQ ID NO: 134 and each heavy chain comprises the amino acid sequence of SEQ ID NO: 135.

In yet another embodiment, provided herein is a fusion protein comprising first and second IL-2 domains, each of SEQ ID NO: 60, an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein each light chain comprises the amino acid sequence of SEQ ID NO: 134 and each heavy chain comprises the amino acid sequence of SEQ ID NO: 135.

In yet another embodiment, provided herein is a fusion protein comprising first and second IL-2 domains, each of SEQ ID NO: 68, an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein each light chain comprises the amino acid sequence of SEQ ID NO: 134 and each heavy chain comprises the amino acid sequence of SEQ ID NO: 135.

In yet another embodiment, provided herein is a fusion protein comprising first and second IL-2 domains, each of SEQ ID NO: 70, an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein each light chain comprises the amino acid sequence of SEQ ID NO: 134 and each heavy chain comprises the amino acid sequence of SEQ ID NO: 135.

In yet another embodiment, provided herein is a fusion protein comprising first and second IL-2 domains, each of SEQ ID NO: 80, an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein each light chain comprises the amino acid sequence of SEQ ID NO: 134 and each heavy chain comprises the amino acid sequence of SEQ ID NO: 135.

In still another embodiment, provided herein is a fusion protein comprising first and second IL-2 domains, each of SEQ ID NO: 256, an intact anti-PD-1 antibody comprising two light chains and first and second heavy chains, and first and second peptide linkers (e.g., each of SEQ ID NO: 215), wherein the N-terminus of the first IL-2 domain is connected to the C-terminus of the first heavy chain via the first peptide linker, and the N-terminus of the second IL-2 domain is connected to the C-terminus of the second heavy chain via the second peptide linker; and wherein each light chain comprises the amino acid sequence of SEQ ID NO: 134 and each heavy chain comprises the amino acid sequence of SEQ ID NO: 135.

In one embodiment, the fusion protein provided herein comprising an intact anti-PD-1 antibody is in a knobs-in-holes configuration. In another embodiment, the fusion protein provided herein comprising an intact anti-PD-1 IgG1 antibody is in a knobs-in-holes configuration. In yet another embodiment, the fusion protein provided herein comprising an intact anti-PD-1 IgG4 antibody is in a knobs-in-holes configuration.

In one embodiment, provided herein is an anti-hPD-1/hIL-2 fusion protein in a knobs-in-holes configuration, comprising a light chain of SEQ ID NO: 136, a heavy chain of SEQ ID NO: 248, and an hIL-2 fused heavy chain of SEQ ID NO: 249 that comprises an hIL-2 domain of SEQ ID NO: 42 and a peptide linker of SEQ ID NO: 215, wherein the N-terminus of the hIL-2 domain is connected to the C-terminus of the second heavy chain via the peptide linker.

In another embodiment, provided herein is an anti-hPD-1/hIL-2 fusion protein in a knobs-in-holes configuration, comprising a light chain of SEQ ID NO: 136, a heavy chain of SEQ ID NO: 248, and an hIL-2 fused heavy chain of SEQ ID NO: 250 that comprises an hIL-2 domain of SEQ ID NO: 54 and a peptide linker of SEQ ID NO: 215, wherein the N-terminus of the hIL-2 domain is connected to the C-terminus of the second heavy chain via the peptide linker.

In yet another embodiment, provided herein is an anti-hPD-1/hIL-2 fusion protein in a knobs-in-holes configuration has a light chain of SEQ ID NO: 136, a heavy chain of SEQ ID NO: 248, and an hIL-2 fused heavy chain of SEQ ID NO: 251 that comprises an hIL-2 domain of SEQ ID NO: 68 and a peptide linker of SEQ ID NO: 215; and wherein the N-terminus of the hIL-2 domain is connected to the C-terminus of the second heavy chain via the peptide linker.

In yet another embodiment, provided herein is an anti-hPD-1/hIL-2 fusion protein in a knobs-in-holes configuration comprising a light chain of SEQ ID NO: 136, a heavy chain of SEQ ID NO: 248, and an hIL-2 fused heavy chain of SEQ ID NO: 252 that comprises an hIL-2 domain of SEQ ID NO: 70 and a peptide linker of SEQ ID NO: 215, wherein the N-terminus of the hIL-2 domain is connected to the C-terminus of the second heavy chain via the peptide linker.

In yet another embodiment, provided herein is an anti-hPD-1/hIL-2 fusion protein in a knobs-in-holes configuration, comprising a light chain of SEQ ID NO: 136, a heavy chain of SEQ ID NO: 248, and an hIL-2 fused heavy chain of SEQ ID NO: 253 that comprises an hIL-2 domain of SEQ ID NO: 80 and a peptide linker of SEQ ID NO: 215, wherein the N-terminus of the hIL-2 domain is connected to the C-terminus of the second heavy chain via the peptide linker.

In still another embodiment, provided herein is an anti-hPD-1/hIL-2 fusion protein in a knobs-in-holes configuration, comprising a light chain of SEQ ID NO: 136, a heavy chain of SEQ ID NO: 248, and an hIL-2 fused heavy chain of SEQ ID NO: 254 that comprising an hIL-2 domain of SEQ ID NO: 50 and a peptide linker of SEQ ID NO: 215, wherein the N-terminus of the hIL-2 domain is connected to the C-terminus of the second heavy chain via the peptide linker.

In one embodiment, provided herein is an anti-hPD-1/hIL-2 fusion protein in a knobs-in-holes configuration, comprising a light chain of SEQ ID NO: 136, a heavy chain of SEQ ID NO: 248, and an hIL-2 fused heavy chain of SEQ ID NO: 249, 250, 251, 252, 253, or 254.

In another embodiment, provided herein is an anti-hPD-1/hIL-2 fusion protein in a knobs-in-holes configuration, comprising a light chain of SEQ ID NO: 136, a heavy chain of SEQ ID NO: 257, and an hIL-2 fused heavy chain of SEQ ID NO: 258, 259, 260, 261, 262, or 263.

In yet another embodiment, provided herein is an anti-hPD-1/hIL-2 fusion protein in a knobs-in-holes configuration, comprising a light chain of SEQ ID NO: 136, a heavy chain of SEQ ID NO: 264, and an hIL-2 fused heavy chain of SEQ ID NO: 202, 265, 266, 267, 268, or 269.

In yet another embodiment, provided herein is an anti-hPD-1/hIL-2 fusion protein in a knobs-in-holes configuration, comprising a light chain of SEQ ID NO: 118, a heavy chain of SEQ ID NO: 201, and an hIL-2 fused heavy chain of SEQ ID NO: 203 or 204.

In still another embodiment, provided herein is an anti-hPD-1/hIL-2 fusion protein in a knobs-in-holes configuration, comprising a light chain of SEQ ID NO: 118, and an hIL-2 fused heavy chain of SEQ ID NO: 205.

### Interleukin-2 Domain

In one embodiment, the IL-2 domain in a fusion protein provided herein comprises, as set forth in SEQ ID NO: 1, 2, 3, 4, or 5, (i) an amino acid substitution at position K8, K9, Q13, E15, H16, L19, D20, M23, K32, P34, K35, L36, T37, R38, M39, L40, T41, F42, K43, F44, Y45, M46, P47, E61, E62, L63, K64, P65, L66, E67, E68, V69, L70, N71, L72, A73, Q74, K76, H79, R81, D84, S87, N88, V91, 192, E95, Y107, D109, T111, S127, or S130; (ii) a disulfide bond formed between two amino acid residues from positions N30 to L80; or (iii) a replacement of the amino acid residues from positions N29 to A50 with a peptide comprising an amino acid sequence of an IL-15 hinge or a fragment thereof (“an IL-15 hinge fragment-containing peptide”).

In another embodiment, the IL-2 domain in a fusion protein provided herein comprises: (i) a replacement of the amino acid residues from positions N29 to A50 with an IL-15 hinge fragment-containing peptide; (ii) an amino acid substitution at position E15, H16, D20, K32, K76, S87, N88, or I92; (iii) an amino acid substitution at a position from R38 to Y45; and/or (iv) a disulfide bond formed between two amino acid residues from positions N30 to L80.

In yet another embodiment, the IL-2 domain in a fusion protein provided herein comprises, as set forth in SEQ ID NO: 1, 2, 3, 4, or 5, (i) an amino acid substitution at position E15, H16, D20, K32, R38, L40, F42, K76, S87, N88, or I92; (ii) a disulfide bond formed between two amino acid residues from positions N30 to L80; or (iii) a replacement of the amino acid residues from positions N29 to A50 with an IL-15 hinge fragment-containing peptide.

In certain embodiments, the amino acid at position E15 as set forth in SEQ ID NO: 1, 2, 3, 4, or 5 is one of the twenty natural amino acids (i.e., Ala (A), Cys (C), Asp (D), Glu (E), Phe (F), Gly (G), His (H), Ile (I), Lys (K), Leu (L), Met (M), Asn (N), Pro (P), Gln (Q), Arg (R), Ser (S), Thr (T), Val (V), Trp (W), and Tyr (Y)) other than E. In certain embodiments, the amino acid at position E15 as set forth in SEQ ID NO: 1, 2, 3, 4, or 5 is K.

In certain embodiments, the amino acid at position H16 as set forth in SEQ ID NO: 1, 2, 3, 4, or 5 is one of the twenty natural amino acids other than H. In certain embodiments, the amino acid at position H16 as set forth in SEQ ID NO: 1, 2, 3, 4, or 5 is E, F, I, or V. In certain embodiments, the amino acid at position H16 as set forth in SEQ ID NO: 1, 2, 3, 4, or 5 is E, I, or V. In certain embodiments, the amino acid at position H16 as set forth in SEQ ID NO: 1, 2, 3, 4, or 5 is E. In certain embodiments, the amino acid at position H16 as set forth in SEQ ID NO: 1, 2, 3, 4, or 5 is F. In certain embodiments, the amino acid at position H16 as set forth in SEQ ID NO: 1, 2, 3, 4, or 5 is I. In certain embodiments, the amino acid at position H16 as set forth in SEQ ID NO: 1, 2, 3, 4, or 5 is V.

In certain embodiments, the amino acid at position D20 as set forth in SEQ ID NO: 1, 2, 3, 4, or 5 is one of the twenty natural amino acids other than D. In certain embodiments, the amino acid at position D20 as set forth in SEQ ID NO: 1, 2, 3, 4, or 5 is A, E, K, or T. In certain embodiments, the amino acid at position D20 as set forth in SEQ ID NO: 1, 2, 3, 4, or 5 is A. In certain embodiments, the amino acid at position D20 as set forth in SEQ ID NO: 1, 2, 3, 4, or 5 is E. In certain embodiments, the amino acid at position D20 as set forth in SEQ ID NO: 1, 2, 3, 4, or 5 is K. In certain embodiments, the amino acid at position D20 as set forth in SEQ ID NO: 1, 2, 3, 4, or 5 is T.

In certain embodiments, the amino acid at position K32 as set forth in SEQ ID NO: 1, 2, 3, 4, or 5 is one of the twenty natural amino acids other than K. In certain embodiments, the amino acid at position K32 as set forth in SEQ ID NO: 1, 2, 3, 4, or 5 is D, E, or Q. In certain embodiments, the amino acid at position K32 as set forth in SEQ ID NO: 1, 2, 3, 4, or 5 is D. In certain embodiments, the amino acid at position K32 as set forth in SEQ ID NO: 1, 2, 3, 4, or 5 is E. In certain embodiments, the amino acid at position K32 as set forth in SEQ ID NO: 1, 2, 3, 4, or 5 is Q.

In certain embodiments, the amino acid at position R38 as set forth in SEQ ID NO: 1, 2, 3, 4, or 5 is one of the twenty natural amino acids other than R. In certain embodiments, the amino acid at position R38 as set forth in SEQ ID NO: 1, 2, 3, 4, or 5 is E or N. In certain embodiments, the amino acid at position R38 as set forth in SEQ ID NO: 1, 2, 3, 4, or 5 is E. In certain embodiments, the amino acid at position R38 as set forth in SEQ ID NO: 1, 2, 3, 4, or 5 is N.

In certain embodiments, the amino acid at position L40 as set forth in SEQ ID NO: 1, 2, 3, 4, or 5 is one of the twenty natural amino acids other than L. In certain embodiments, the amino acid at position L40 as set forth in SEQ ID NO: 1, 2, 3, 4, or 5 is S or T. In certain embodiments, the amino acid at position L40 as set forth in SEQ ID NO: 1, 2, 3, 4, or 5 is S. In certain embodiments, the amino acid at position L40 as set forth in SEQ ID NO: 1, 2, 3, 4, or 5 is T.

In certain embodiments, the amino acid at position F42 as set forth in SEQ ID NO: 1, 2, 3, 4, or 5 is one of the twenty natural amino acids other than F. In certain embodiments, the amino acid at position F42 as set forth in SEQ ID NO: 1, 2, 3, 4, or 5 is A, C, K, or N. In certain embodiments, the amino acid at position F42 as set forth in SEQ ID NO: 1, 2, 3, 4, or 5 is A or K. In certain embodiments, the amino acid at position F42 as set forth in SEQ ID NO: 1, 2, 3, 4, or 5 is A. In certain embodiments, the amino acid at position F42 as set forth in SEQ ID NO: 1, 2, 3, 4, or 5 is C. In certain embodiments, the amino acid at position F42 as set forth in SEQ ID NO: 1, 2, 3, 4, or 5 is K. In certain embodiments, the amino acid at position F42 as set forth in SEQ ID NO: 1, 2, 3, 4, or 5 is N.

In certain embodiments, the amino acid at position K76 as set forth in SEQ ID NO: 1, 2, 3, 4, or 5 is one of the twenty natural amino acids other than K. In certain embodiments, the amino acid at position K76 as set forth in SEQ ID NO: 1, 2, 3, 4, or 5 is D, E, or Q. In certain embodiments, the amino acid at position K76 as set forth in SEQ ID NO: 1, 2, 3, 4, or 5 is D. In certain embodiments, the amino acid at position K76 as set forth in SEQ ID NO: 1, 2, 3, 4, or 5 is E. In certain embodiments, the amino acid at position K76 as set forth in SEQ ID NO: 1, 2, 3, 4, or 5 is Q.

In certain embodiments, the amino acid at position S87 as set forth in SEQ ID NO: 1, 2, 3, 4, or 5 is one of the twenty natural amino acids other than S. In certain embodiments, the amino acid at position S87 as set forth in SEQ ID NO: 1, 2, 3, 4, or 5 is D or E. In certain embodiments, the amino acid at position S87 as set forth in SEQ ID NO: 1, 2, 3, 4, or 5 is D. In certain embodiments, the amino acid at position S87 as set forth in SEQ ID NO: 1, 2, 3, 4, or 5 is E.

In certain embodiments, the amino acid at position N88 as set forth in SEQ ID NO: 1, 2, 3, 4, or 5 is one of the twenty natural amino acids other than N. In certain embodiments, the amino acid at position N88 as set forth in SEQ ID NO: 1, 2, 3, 4, or 5 is A.

In certain embodiments, the amino acid at position I92 as set forth in SEQ ID NO: 1, 2, 3, 4, or 5 is one of the twenty natural amino acids other than I. In certain embodiments, the amino acid at position I92 as set forth in SEQ ID NO: 1, 2, 3, 4, or 5 is A, D, E, or G. In certain embodiments, the amino acid at position I92 as set forth in SEQ ID NO: 1, 2, 3, 4, or 5 is A. In certain embodiments, the amino acid at position I92 as set forth in SEQ ID NO: 1, 2, 3, 4, or 5 is D. In certain embodiments, the amino acid at position I92 as set forth in SEQ ID NO: 1, 2, 3, 4, or 5 is E. In certain embodiments, the amino acid at position I92 as set forth in SEQ ID NO: 1, 2, 3, 4, or 5 is G.

In yet another embodiment, the IL-2 domain in a fusion protein provided herein comprises, as set forth in SEQ ID NO: 1, 2, 3, 4, or 5, (i) an amino acid substitution of K8D, K8E, K8Q, K9D, K9E, K9Q, Q13E, Q13N, E15K, E15Q, E15V, H16E, H16F, H16I, H16V, L19S, D20A, D20E, D20K, D20T, M23K, K32D, K32E, K32Q, P34N, K35N, L36S, L36T, T37N, R38E, R38N, M39N, L40S, L40T, T41N, F42A, F42C, F42K, F42N, K43N, F44N, Y45N, M46S, M46T, P47S, P47T, E61N, E62N, L63S, L63T, K64N, P65N, L66N, E67S, E67T, E68N, V69C, V69N, L70S, L70T, N71S, N71T, L72N, A73S, A73T, Q74S, Q74T, K76D, K76E, K76Q, H79D, H79E, H79Q, R81D, R81E, R81Q, D84T, S87D, S87E, N88A, V91I, I92A, I92D, I92E, I92G, I92L, E95K, E95N, E95Q, Y107N, D109N, T111S, S127A, S127E, S127F, S127W, S130A, S130E, S130F, or S130W; (ii) a disulfide bond formed between two different amino acid residues, each independently at position K35, R38, F42, Y45, E62, V69, or L72; or (iii) a replacement of the amino acid residues from positions N29 to A50 having an amino acid sequence of SEQ ID NO: 38 with an IL-15 hinge fragment-containing peptide.

In yet another embodiment, the IL-2 domain in a fusion protein provided herein comprises, as set forth in SEQ ID NO: 1, 2, 3, 4, or 5, (i) an amino acid substitution of E15K, H16E, H16F, H16I, H16V, D20A, D20E, D20K, D20T, K32E, R38E, R38N, L40S, L40T, F42A, F42K, F42N, K76D, K76E, K76Q, S87D, S87E, N88A, I92A, I92D, I92E, or I92G; (ii) a disulfide bond formed between F42C and V69C; or (iii) a replacement of the amino acid residues from positions N29 to L40 having an amino acid sequence of SEQ ID NO: 39 with an IL-15 hinge fragment-containing peptide.

In one embodiment, the IL-15 hinge fragment-containing peptide comprises an amino acid sequence of SEQ ID NO: 40 or 41. In another embodiment, the IL-15 hinge fragment-containing peptide comprises an amino acid sequence of SEQ ID NO: 40. In yet another embodiment, the IL-15 hinge fragment-containing peptide comprises an amino acid sequence of SEQ ID NO: 41.

In still another embodiment, the IL-2 domain in a fusion protein provided herein comprises, as set forth in SEQ ID NO: 1, 2, 3, 4, or 5, (i) an amino acid substitution of E15K, H16E, H16F, H16I, H16V, D20A, D20E, D20K, D20T, K32E, R38E, R38N, L40T, F42A, F42K, K76E, S87D, N88A, I92A, I92D, I92E, or I92G; (ii) a disulfide bond formed between F42C and V69C; or (iii) a replacement of the amino acid residues from positions N29 to L40 having an amino acid sequence of SEQ ID NO: 39 with an IL-15 hinge fragment-containing peptide having an amino acid sequence of SEQ ID NO: 40 or 41.

In one embodiment, the IL-2 domain in a fusion protein provided herein comprises an amino acid substitution at position K8, K9, Q13, E15, H16, L19, D20, M23, K32, P34, K35, L36, T37, R38, M39, L40, T41, F42, K43, F44, Y45, M46, P47, E61, E62, L63, K64, P65, L66, E67, E68, V69, L70, N71, L72, A73, Q74, K76, H79, R81, D84, S87, N88, V91, I92, E95, Y107, D109, T111, S127, or S130 as set forth in SEQ ID NO: 1, 2, 3, 4, or 5.

In another embodiment, the IL-2 domain in a fusion protein provided herein comprises an amino acid substitution of K8D, K8E, K8Q, K9D, K9E, K9Q, Q13E, Q13N, E15K, E15Q, E15V, H16E, H16F, H16I, H16V, L19S, D20A, D20E, D20K, D20T, M23K, K32D, K32E, K32Q, P34N, K35N, L36S, L36T, T37N, R38E, R38N, M39N, L40S, L40T, T41N, F42A, F42C, F42K, F42N, K43N, F44N, Y45N, M46S, M46T, P47S, P47T, E61N, E62N, L63S, L63T, K64N, P65N, L66N, E67S, E67T, E68N, V69C, V69N, L70S, L70T, N71S, N71T, L72N, A73S, A73T, Q74S, Q74T, K76D, K76E, K76Q, H79D, H79E, H79Q, R81D, R81E, R81Q, D84T, S87D, S87E, N88A, V91I, I92A, I92D, I92E, I92G, I92L, E95K, E95N, E95Q, Y107N, D109N, T111S, S127A, S127E, S127F, S127W, S130A, S130E, S130F, or S130W as set forth in SEQ ID NO: 1, 2, 3, 4, or 5.

In yet another embodiment, the IL-2 domain in a fusion protein provided herein comprises an amino acid substitution at position E15, H16, D20, K32, R38, L40, F42, K76, S87, N88, or I92 as set forth in SEQ ID NO: 1, 2, 3, 4, or 5.

In yet another embodiment, the IL-2 domain in a fusion protein provided herein comprises an amino acid substitution of E15K, H16E, H16F, H16I, H16V, D20A, D20E, D20K, D20T, K32E, R38E, R38N, L40S, L40T, F42A, F42K, F42N, K76D, K76E, K76Q, S87D, S87E, N88A, I92A, I92D, I92E, or I92G as set forth in SEQ ID NO: 1, 2, 3, 4, or 5.

In still another embodiment, the IL-2 domain in a fusion protein provided herein comprises an amino acid substitution of E15K, H16E, H16F, H16I, H16V, D20A, D20E, D20K, D20T, K32E, R38E, R38N, L40T, F42A, F42K, K76E, S87D, N88A, I92A, I92D, I92E, or I92G as set forth in SEQ ID NO: 1, 2, 3, 4, or 5.

In one embodiment, the IL-2 domain in a fusion protein provided herein comprises an amino acid substitution at position E15 as set forth in SEQ ID NO: 1, 2, 3, 4, or 5. In another embodiment, the IL-2 domain in a fusion protein provided herein comprises an amino acid substitution of E15K as set forth in SEQ ID NO: 1, 2, 3, 4, or 5.

In one embodiment, the IL-2 domain in a fusion protein provided herein comprises an amino acid substitution at position H16 as set forth in SEQ ID NO: 1, 2, 3, 4, or 5. In another embodiment, the IL-2 domain in a fusion protein provided herein comprises an amino acid substitution of H16E, H16F, H16I, or H16V as set forth in SEQ ID NO: 1, 2, 3, 4, or 5. In yet another embodiment, the IL-2 domain in a fusion protein provided herein comprises an amino acid substitution of H16E, H16I, or H16V as set forth in SEQ ID NO: 1, 2, 3, 4, or 5. In yet another embodiment, the IL-2 domain in a fusion protein provided herein comprises an amino acid substitution of H16E as set forth in SEQ ID NO: 1, 2, 3, 4, or 5. In yet another embodiment, the IL-2 domain in a fusion protein provided herein comprises an amino acid substitution of H16F as set forth in SEQ ID NO: 1, 2, 3, 4, or 5. In yet another embodiment, the IL-2 domain in a fusion protein provided herein comprises an amino acid substitution of H16I as set forth in SEQ ID NO: 1, 2, 3, 4, or 5. In still another embodiment, the IL-2 domain in a fusion protein provided herein comprises an amino acid substitution of H16V as set forth in SEQ ID NO: 1, 2, 3, 4, or 5.

In one embodiment, the IL-2 domain in a fusion protein provided herein comprises an amino acid substitution at position D20 as set forth in SEQ ID NO: 1, 2, 3, 4, or 5. In another embodiment, the IL-2 domain in a fusion protein provided herein comprises an amino acid substitution of D20A, D20E, D20K, or D20T as set forth in SEQ ID NO: 1, 2, 3, 4, or 5. In yet another embodiment, the IL-2 domain in a fusion protein provided herein comprises an amino acid substitution of D20A as set forth in SEQ ID NO: 1, 2, 3, 4, or 5. In yet another embodiment, the IL-2 domain in a fusion protein provided herein comprises an amino acid substitution of D20E as set forth in SEQ ID NO: 1, 2, 3, 4, or 5. In yet another embodiment, the IL-2 domain in a fusion protein provided herein comprises an amino acid substitution of D20K as set forth in SEQ ID NO: 1, 2, 3, 4, or 5. In still another embodiment, the IL-2 domain in a fusion protein provided herein comprises an amino acid substitution of D20T as set forth in SEQ ID NO: 1, 2, 3, 4, or 5.

In one embodiment, the IL-2 domain in a fusion protein provided herein comprises an amino acid substitution at position K32 as set forth in SEQ ID NO: 1, 2, 3, 4, or 5. In another embodiment, the IL-2 domain in a fusion protein provided herein comprises an amino acid substitution of K32E as set forth in SEQ ID NO: 1, 2, 3, 4, or 5.

In one embodiment, the IL-2 domain in a fusion protein provided herein comprises an amino acid substitution at position R38 as set forth in SEQ ID NO: 1, 2, 3, 4, or 5. In another embodiment, the IL-2 domain in a fusion protein provided herein comprises an amino acid substitution of R38E or R38N as set forth in SEQ ID NO: 1, 2, 3, 4, or 5. In yet another embodiment, the IL-2 domain in a fusion protein provided herein comprises an amino acid substitution of R38E as set forth in SEQ ID NO: 1, 2, 3, 4, or 5. In still another embodiment, the IL-2 domain in a fusion protein provided herein comprises an amino acid substitution of R38N as set forth in SEQ ID NO: 1, 2, 3, 4, or 5.

In one embodiment, the IL-2 domain in a fusion protein provided herein comprises an amino acid substitution at position L40 as set forth in SEQ ID NO: 1, 2, 3, 4, or 5. In another embodiment, the IL-2 domain in a fusion protein provided herein comprises an amino acid substitution of L40S or L40T as set forth in SEQ ID NO: 1, 2, 3, 4, or 5. In yet another embodiment, the IL-2 domain in a fusion protein provided herein comprises an amino acid substitution of L40S as set forth in SEQ ID NO: 1, 2, 3, 4, or 5. In still another embodiment, the IL-2 domain in a fusion protein provided herein comprises an amino acid substitution of L40T as set forth in SEQ ID NO: 1, 2, 3, 4, or 5.

In one embodiment, the IL-2 domain in a fusion protein provided herein comprises an amino acid substitution at position F42 as set forth in SEQ ID NO: 1, 2, 3, 4, or 5. In another embodiment, the IL-2 domain in a fusion protein provided herein comprises an amino acid substitution of F42A, F42C, or F42K as set forth in SEQ ID NO: 1, 2, 3, 4, or 5. In yet another embodiment, the IL-2 domain in a fusion protein provided herein comprises an amino acid substitution of F42A or F42K as set forth in SEQ ID NO: 1, 2, 3, 4, or 5. In yet another embodiment, the IL-2 domain in a fusion protein provided herein comprises an amino acid substitution of F42A as set forth in SEQ ID NO: 1, 2, 3, 4, or 5. In yet another embodiment, the IL-2 domain in a fusion protein provided herein comprises an amino acid substitution of F42C as set forth in SEQ ID NO: 1, 2, 3, 4, or 5. In still another embodiment, the IL-2 domain in a fusion protein provided herein comprises an amino acid substitution of F42K as set forth in SEQ ID NO: 1, 2, 3, 4, or 5.

In one embodiment, the IL-2 domain in a fusion protein provided herein comprises an amino acid substitution at position K76 as set forth in SEQ ID NO: 1, 2, 3, 4, or 5. In another embodiment, the IL-2 domain in a fusion protein provided herein comprises an amino acid substitution of K76E as set forth in SEQ ID NO: 1, 2, 3, 4, or 5.

In one embodiment, the IL-2 domain in a fusion protein provided herein comprises an amino acid substitution at position S87 as set forth in SEQ ID NO: 1, 2, 3, 4, or 5. In another embodiment, the IL-2 domain in a fusion protein provided herein comprises an amino acid substitution of S87D as set forth in SEQ ID NO: 1, 2, 3, 4, or 5.

In one embodiment, the IL-2 domain in a fusion protein provided herein comprises an amino acid substitution at position N88 as set forth in SEQ ID NO: 1, 2, 3, 4, or 5. In another embodiment, the IL-2 domain in a fusion protein provided herein comprises an amino acid substitution of N88A as set forth in SEQ ID NO: 1, 2, 3, 4, or 5.

In one embodiment, the IL-2 domain in a fusion protein provided herein comprises an amino acid substitution at position I92 as set forth in SEQ ID NO: 1, 2, 3, 4, or 5. In another embodiment, the IL-2 domain in a fusion protein provided herein comprises an amino acid substitution of I92A, I92D, I92E, or I92G as set forth in SEQ ID NO: 1, 2, 3, 4, or 5. In yet another embodiment, the IL-2 domain in a fusion protein provided herein comprises an amino acid substitution of I92A as set forth in SEQ ID NO: 1, 2, 3, 4, or 5. In yet another embodiment, the IL-2 domain in a fusion protein provided herein comprises an amino acid substitution of I92D as set forth in SEQ ID NO: 1, 2, 3, 4, or 5. In yet another embodiment, the IL-2 domain in a fusion protein provided herein comprises an amino acid substitution of I92E as set forth in SEQ ID NO: 1, 2, 3, 4, or 5. In still another embodiment, the IL-2 domain in a fusion protein provided herein comprises an amino acid substitution of I92G as set forth in SEQ ID NO: 1, 2, 3, 4, or 5.

In one embodiment, the IL-2 domain in a fusion protein provided herein comprises, as set forth in SEQ ID NO: 1, 2, 3, 4, or 5, amino acid substitutions at positions R38 and L40, and optionally an amino acid substitution at position E15, H16, D20, K32, K76, S87, N88, or I92. In another embodiment, the IL-2 domain in a fusion protein provided herein comprises, as set forth in SEQ ID NO: 1, 2, 3, 4, or 5, amino acid substitutions at positions R38 and L40, and optionally an amino acid substitution at position E15, H16, D20, K76, S87, N88, or I92. In yet another embodiment, the IL-2 domain in a fusion protein provided herein comprises, as set forth in SEQ ID NO: 1, 2, 3, 4, or 5, amino acid substitutions at positions E15, R38, and L40, and optionally an amino acid substitution at position H16, D20, K76, S87, N88, or I92. In yet another embodiment, the IL-2 domain in a fusion protein provided herein comprises, as set forth in SEQ ID NO: 1, 2, 3, 4, or 5, amino acid substitutions at positions R38, L40, and K76, and optionally an amino acid substitution at position E15, H16, D20, S87, N88, or I92. In still another embodiment, the IL-2 domain in a fusion protein provided herein comprises, as set forth in SEQ ID NO: 1, 2, 3, 4, or 5, amino acid substitutions at positions E15, R38, L40, and K76, and optionally an amino acid substitution at position H16, D20, S87, N88, or I92.

In one embodiment, the IL-2 domain in a fusion protein provided herein comprises, as set forth in SEQ ID NO: 1, 2, 3, 4, or 5, amino acid substitutions of R38N and L40S or L40T, and optionally an amino acid substitution of E15K, H16E, H16F, H16I, H16V, D20A, D20E, D20K, D20T, K32E, K76E, S87D, N88A, I92A, I92D, I92E, or I92G. In another embodiment, the IL-2 domain in a fusion protein provided herein comprises, as set forth in SEQ ID NO: 1, 2, 3, 4, or 5, amino acid substitutions of R38N and L40S or L40T, and optionally an amino acid substitution of E15K, H16E, H16I, H16V, D20A, D20E, D20K, D20T, K76E, S87D, N88A, I92A, I92D, I92E, or I92G. In yet another embodiment, the IL-2 domain in a fusion protein provided herein comprises, as set forth in SEQ ID NO: 1, 2, 3, 4, or 5, amino acid substitutions of E15K, R38N and L40S or L40T, and optionally an amino acid substitution of H16E, H16F, H16I, H16V, D20A, D20E, D20K, D20T, K76E, S87D, N88A, I92A, I92D, I92E, or I92G. In yet another embodiment, the IL-2 domain in a fusion protein provided herein comprises, as set forth in SEQ ID NO: 1, 2, 3, 4, or 5, amino acid substitutions of R38N, L40S or L40T, and K76E, and optionally an amino acid substitution of E15K, H16E, H16F, H16I, H16V, D20A, D20E, D20K, D20T, S87D, N88A, I92A, I92D, I92E, or I92G. In still another embodiment, the IL-2 domain in a fusion protein provided herein comprises, as set forth in SEQ ID NO: 1, 2, 3, 4, or 5, amino acid substitutions of E15K, R38N, L40S or L40T, and K76E, and optionally an amino acid substitution of H16E, H16F, H16I, H16V, D20A, D20E, D20K, D20T, S87D, N88A, I92A, I92D, I92E, or I92G.

In one embodiment, the IL-2 domain in a fusion protein provided herein comprises, as set forth in SEQ ID NO: 1, 2, 3, 4, or 5, amino acid substitutions of R38N and L40S, and optionally an amino acid substitution of E15K, H16E, H16F, H16I, H16V, D20A, D20E, D20K, D20T, K76E, S87D, N88A, I92A, I92D, I92E, or I92G. In another embodiment, the IL-2 domain in a fusion protein provided herein comprises, as set forth in SEQ ID NO: 1, 2, 3, 4, or 5, amino acid substitutions of E15K, R38N and L40S, and optionally an amino acid substitution of H16E, H16F, H16I, H16V, D20A, D20E, D20K, D20T, K76E, S87D, N88A, I92A, I92D, I92E, or I92G. In yet another embodiment, the IL-2 domain in a fusion protein provided herein comprises, as set forth in SEQ ID NO: 1, 2, 3, 4, or 5, amino acid substitutions of R38N, L40S, and K76E, and optionally an amino acid substitution of E15K, H16E, H16F, H16I, H16V, D20A, D20E, D20K, D20T, S87D, N88A, I92A, I92D, I92E, or I92G. In still another embodiment, the IL-2 domain in a fusion protein provided herein comprises, as set forth in SEQ ID NO: 1, 2, 3, 4, or 5, amino acid substitutions of E15K, R38N, L40S, and K76E, and optionally an amino acid substitution of H16E, H16F, H16I, H16V, D20A, D20E, D20K, D20T, S87D, N88A, I92A, I92D, I92E, or I92G.

In one embodiment, the IL-2 domain in a fusion protein provided herein comprises, as set forth in SEQ ID NO: 1, 2, 3, 4, or 5, amino acid substitutions of R38N and L40T, and optionally an amino acid substitution of E15K, H16E, H16F, H16I, H16V, D20A, D20E, D20K, D20T, K76E, S87D, N88A, I92A, I92D, I92E, or I92G. In another embodiment, the IL-2 domain in a fusion protein provided herein comprises, as set forth in SEQ ID NO: 1, 2, 3, 4, or 5, amino acid substitutions of E15K, R38N and L40T, and optionally an amino acid substitution of H16E, H16F, H16I, H16V, D20A, D20E, D20K, D20T, K76E, S87D, N88A, I92A, I92D, I92E, or I92G. In yet another embodiment, the IL-2 domain in a fusion protein provided herein comprises, as set forth in SEQ ID NO: 1, 2, 3, 4, or 5, amino acid substitutions of R38N, L40T, and K76E, and optionally an amino acid substitution of E15K, H16E, H16F, H16I, H16V, D20A, D20E, D20K, D20T, S87D, N88A, I92A, I92D, I92E, or I92G. In still another embodiment, the IL-2 domain in a fusion protein provided herein comprises, as set forth in SEQ ID NO: 1, 2, 3, 4, or 5, amino acid substitutions of E15K, R38N, L40T, and K76E, and optionally an amino acid substitution of H16E, H16F, H16I, H16V, D20A, D20E, D20K, D20T, S87D, N88A, I92A, I92D, I92E, or I92G.

In one embodiment, the IL-2 domain in a fusion protein provided herein comprises amino acid substitutions at positions R38 and L40 as set forth in SEQ ID NO: 1, 2, 3, 4, or 5. In another embodiment, the IL-2 domain in a fusion protein provided herein comprises amino acid substitutions of R38N and L40S or L40T as set forth in SEQ ID NO: 1, 2, 3, 4, or 5. In yet another embodiment, the IL-2 domain in a fusion protein provided herein comprises amino acid substitutions of R38N and L40S as set forth in SEQ ID NO: 1, 2, 3, 4, or 5. In still another embodiment, the IL-2 domain in a fusion protein provided herein comprises amino acid substitutions of R38N and L40T as set forth in SEQ ID NO: 1, 2, 3, 4, or 5.

In one embodiment, the IL-2 domain in a fusion protein provided herein comprises amino acid substitutions at positions E15, R38, and L40 as set forth in SEQ ID NO: 1, 2, 3, 4, or 5. In another embodiment, the IL-2 domain in a fusion protein provided herein comprises amino acid substitutions of E15K, R38N, and L40S or L40T as set forth in SEQ ID NO: 1, 2, 3, 4, or 5. In yet another embodiment, the IL-2 domain in a fusion protein provided herein comprises amino acid substitutions of E15K, R38N, and L40S as set forth in SEQ ID NO: 1, 2, 3, 4, or 5. In still another embodiment, the IL-2 domain in a fusion protein provided herein comprises amino acid substitutions of E15K, R38N, and L40T as set forth in SEQ ID NO: 1, 2, 3, 4, or 5.

In one embodiment, the IL-2 domain in a fusion protein provided herein comprises amino acid substitutions at positions E15, R38, L40, and K76 as set forth in SEQ ID NO: 1, 2, 3, 4, or 5. In another embodiment, the IL-2 domain in a fusion protein provided herein comprises amino acid substitutions of E15K, R38N, L40S or L40T, and K76E as set forth in SEQ ID NO: 1, 2, 3, 4, or 5. In another embodiment, the IL-2 domain in a fusion protein provided herein comprises amino acid substitutions of E15K, R38N, L40S, and K76E as set forth in SEQ ID NO: 1, 2, 3, 4, or 5. In still another embodiment, the IL-2 domain in a fusion protein provided herein comprises amino acid substitutions of E15K, R38N, L40T, and K76E as set forth in SEQ ID NO: 1, 2, 3, 4, or 5.

In one embodiment, the IL-2 domain in a fusion protein provided herein comprises an amino acid sequence of any one of SEQ ID NOs: 6 to 13.

In one embodiment, the IL-2 domain in a fusion protein provided is glycosylated. In another embodiment, the IL-2 domain in a fusion protein provided is N-glycosylated. In yet another embodiment, the IL-2 domain in the fusion protein provided herein is glycosylate at the nitrogen in the side chain of an asparagine residue. In still another embodiment, the IL-2 domain in a fusion protein provided comprises an amino acid substitution of R38N that is N-glycosylated.

In one embodiment, the IL-2 domain in a fusion protein provided herein is an N-glycosylated IL-2 domain comprising, as set forth in SEQ ID NO: 1, 2, 3, 4, or 5, amino acid substitutions at positions R38 and L40, and optionally an amino acid substitution at position E15, H16, D20, E32, K76, S87, N88, or I92. In another embodiment, the IL-2 domain in a fusion protein provided herein is an N-glycosylated IL-2 domain comprising, as set forth in SEQ ID NO: 1, 2, 3, 4, or 5, amino acid substitutions at positions R38 and L40, and optionally an amino acid substitution at position E15, H16, D20, K76, S87, N88, or I92. In yet another embodiment, the IL-2 domain in a fusion protein provided herein is an N-glycosylated IL-2 domain comprising, as set forth in SEQ ID NO: 1, 2, 3, 4, or 5, amino acid substitutions at positions E15, R38, and L40, and optionally an amino acid substitution at position H16, D20, K76, S87, N88, or I92. In yet another embodiment, the IL-2 domain in a fusion protein provided herein is an N-glycosylated IL-2 domain comprising, as set forth in SEQ ID NO: 1, 2, 3, 4, or 5, amino acid substitutions at positions R38, L40, and K76, and optionally an amino acid substitution at position E15, H16, D20, S87, N88, or I92. In still another embodiment, the IL-2 domain in a fusion protein provided herein is an N-glycosylated IL-2 domain comprising, as set forth in SEQ ID NO: 1, 2, 3, 4, or 5, amino acid substitutions at positions E15, R38, L40, and K76, and optionally an amino acid substitution at position H16, D20, S87, N88, or I92.

In one embodiment, the IL-2 domain in a fusion protein provided herein is an N-glycosylated IL-2 domain comprising, as set forth in SEQ ID NO: 1, 2, 3, 4, or 5, amino acid substitutions of R38N and L40S or L40T, and optionally an amino acid substitution of E15K, H16E, H16F, H16I, H16V, D20A, D20E, D20K, D20T, E32K, K76E, S87D, N88A, I92A, I92D, I92E, or I92G. In another embodiment, the IL-2 domain in a fusion protein provided herein is an N-glycosylated IL-2 domain comprising, as set forth in SEQ ID NO: 1, 2, 3, 4, or 5, amino acid substitutions of R38N and L40S or L40T, and optionally an amino acid substitution of E15K, H16E, H16F, H16I, H16V, D20A, D20E, D20K, D20T, K76E, S87D, N88A, I92A, I92D, I92E, or I92G. In yet another embodiment, the IL-2 domain in a fusion protein provided herein is an N-glycosylated IL-2 domain comprising, as set forth in SEQ ID NO: 1, 2, 3, 4, or 5, amino acid substitutions of E15K, R38N and L40S or L40T, and optionally an amino acid substitution of H16E, H16F, H16I, H16V, D20A, D20E, D20K, D20T, K76E, S87D, N88A, I92A, I92D, I92E, or I92G. In yet another embodiment, the IL-2 domain in a fusion protein provided herein is an N-glycosylated IL-2 domain comprising, as set forth in SEQ ID NO: 1, 2, 3, 4, or 5, amino acid substitutions of R38N, L40S or L40T, and K76E, and optionally an amino acid substitution of E15K, H16E, H16F, H16I, H16V, D20A, D20E, D20K, D20T, S87D, N88A, I92A, I92D, I92E, or I92G. In still another embodiment, the IL-2 domain in a fusion protein provided herein is an N-glycosylated IL-2 domain comprising, as set forth in SEQ ID NO: 1, 2, 3, 4, or 5, amino acid substitutions of E15K, R38N, L40S or L40T, and K76E, and optionally an amino acid substitution of H16E, H16F, H16I, H16V, D20A, D20E, D20K, D20T, S87D, N88A, I92A, I92D, I92E, or I92G.

In one embodiment, the IL-2 domain in a fusion protein provided herein is an N-glycosylated IL-2 domain comprising, as set forth in SEQ ID NO: 1, 2, 3, 4, or 5, amino acid substitutions of R38N and L40S, and optionally an amino acid substitution of E15K, H16E, H16F, H16I, H16V, D20A, D20E, D20K, D20T, K76E, S87D, N88A, I92A, I92D, I92E, or I92G. In another embodiment, the IL-2 domain in a fusion protein provided herein is an N-glycosylated IL-2 domain comprising, as set forth in SEQ ID NO: 1, 2, 3, 4, or 5, amino acid substitutions of E15K, R38N and L40S, and optionally an amino acid substitution of H16E, H16F, H16I, H16V, D20A, D20E, D20K, D20T, K76E, S87D, N88A, I92A, I92D, I92E, or I92G. In yet another embodiment, the IL-2 domain in a fusion protein provided herein is an N-glycosylated IL-2 domain comprising, as set forth in SEQ ID NO: 1, 2, 3, 4, or 5, amino acid substitutions of R38N, L40S, and K76E, and optionally an amino acid substitution of E15K, H16E, H16F, H16I, H16V, D20A, D20E, D20K, D20T, S87D, N88A, I92A, I92D, I92E, or I92G. In still another embodiment, the IL-2 domain in a fusion protein provided herein is an N-glycosylated IL-2 domain comprising, as set forth in SEQ ID NO: 1, 2, 3, 4, or 5, amino acid substitutions of E15K, R38N, L40S, and K76E, and optionally an amino acid substitution of H16E, H16F, H16I, H16V, D20A, D20E, D20K, D20T, S87D, N88A, I92A, I92D, I92E, or I92G.

In one embodiment, the IL-2 domain in a fusion protein provided herein is an N-glycosylated IL-2 domain comprising, as set forth in SEQ ID NO: 1, 2, 3, 4, or 5, amino acid substitutions of R38N and L40T, and optionally an amino acid substitution of E15K, H16E, H16F, H16I, H16V, D20A, D20E, D20K, D20T, K76E, S87D, N88A, I92A, I92D, I92E, or I92G. In another embodiment, the IL-2 domain in a fusion protein provided herein is an N-glycosylated IL-2 domain comprising, as set forth in SEQ ID NO: 1, 2, 3, 4, or 5, amino acid substitutions of E15K, R38N and L40T, and optionally an amino acid substitution of H16E, H16F, H16I, H16V, D20A, D20E, D20K, D20T, K76E, S87D, N88A, I92A, I92D, I92E, or I92G. In yet another embodiment, the IL-2 domain in a fusion protein provided herein is an N-glycosylated IL-2 domain comprising, as set forth in SEQ ID NO: 1, 2, 3, 4, or 5, amino acid substitutions of R38N, L40T, and K76E, and optionally an amino acid substitution of E15K, H16E, H16F, H16I, H16V, D20A, D20E, D20K, D20T, S87D, N88A, I92A, I92D, I92E, or I92G. In still another embodiment, the IL-2 domain in a fusion protein provided herein is an N-glycosylated IL-2 domain comprising, as set forth in SEQ ID NO: 1, 2, 3, 4, or 5, amino acid substitutions of E15K, R38N, L40T, and K76E, and optionally an amino acid substitution of H16E, H16F, H16I, H16V, D20A, D20E, D20K, D20T, S87D, N88A, I92A, I92D, I92E, or I92G.

In one embodiment, the IL-2 domain in a fusion protein provided herein is an N-glycosylated IL-2 domain comprising, amino acid substitutions at positions R38 and L40 as set forth in SEQ ID NO: 1, 2, 3, 4, or 5. In another embodiment, the IL-2 domain in a fusion protein provided herein is an N-glycosylated IL-2 domain comprising, amino acid substitutions of R38N and L40S or L40T as set forth in SEQ ID NO: 1, 2, 3, 4, or 5. In yet another embodiment, the IL-2 domain in a fusion protein provided herein is an N-glycosylated IL-2 domain comprising, amino acid substitutions of R38N and L40S as set forth in SEQ ID NO: 1, 2, 3, 4, or 5. In still another embodiment, the IL-2 domain in a fusion protein provided herein is an N-glycosylated IL-2 domain comprising, amino acid substitutions of R38N and L40T as set forth in SEQ ID NO: 1, 2, 3, 4, or 5.

In one embodiment, the IL-2 domain in a fusion protein provided herein is an N-glycosylated IL-2 domain comprising, amino acid substitutions at positions E15, R38, and L40 as set forth in SEQ ID NO: 1, 2, 3, 4, or 5. In another embodiment, the IL-2 domain in a fusion protein provided herein is an N-glycosylated IL-2 domain comprising, amino acid substitutions of E15K, R38N, and L40S or L40T as set forth in SEQ ID NO: 1, 2, 3, 4, or 5. In yet another embodiment, the IL-2 domain in a fusion protein provided herein is an N-glycosylated IL-2 domain comprising, amino acid substitutions of E5K, R38N, and L40S as set forth in SEQ ID NO: 1, 2, 3, 4, or 5. In still another embodiment, the IL-2 domain in a fusion protein provided herein is an N-glycosylated IL-2 domain comprising, amino acid substitutions of E15K, R38N, and L40T as set forth in SEQ ID NO: 1, 2, 3, 4, or 5.

In one embodiment, the IL-2 domain in a fusion protein provided herein is an N-glycosylated IL-2 domain comprising, amino acid substitutions at positions E15, R38, L40, and K76 as set forth in SEQ ID NO: 1, 2, 3, 4, or 5. In another embodiment, the IL-2 domain in a fusion protein provided herein is an N-glycosylated IL-2 domain comprising, amino acid substitutions of E15K, R38N, L40S or L40T, and K76E as set forth in SEQ ID NO: 1, 2, 3, 4, or 5. In another embodiment, the IL-2 domain in a fusion protein provided herein is an N-glycosylated IL-2 domain comprising, amino acid substitutions of E15K, R38N, L40S, and K76E as set forth in SEQ ID NO: 1, 2, 3, 4, or 5. In still another embodiment, the IL-2 domain in a fusion protein provided herein is an N-glycosylated IL-2 domain comprising, amino acid substitutions of E15K, R38N, L40T, and K76E as set forth in SEQ ID NO: 1, 2, 3, 4, or 5.

In one embodiment, the N-glycosylated IL-2 domain in a fusion protein provided herein comprises an amino acid sequence of any one of SEQ ID NOs: 6 to 13.

In one embodiment, the N-glycosylated IL-2 domain in the fusion protein provided herein has one glycan. In another embodiment, the N-glycosylated IL-2 domain in the fusion protein provided herein has one glycan attached to the nitrogen in the side chain of an asparagine residue. In yet another embodiment, the N-glycosylated IL-2 domain in the fusion protein provided herein has one glycan attached to the nitrogen in the side chain of an asparagine residue at position R38N.

In one embodiment, the N-glycosylated IL-2 domain in the fusion protein provided herein has two glycans. In another embodiment, the N-glycosylated IL-2 domain in the fusion protein provided herein has two glycans, of which at least one glycan is attached to the nitrogen in the side chain of an asparagine residue. In yet another embodiment, the N-glycosylated IL-2 domain in the fusion protein provided herein has two glycans, each of which is attached to the nitrogen in the side chain of an asparagine residue.

In one embodiment, the N-glycosylated IL-2 domain in a fusion protein provided herein has three glycans. In one embodiment, the glycan is an N-glycan.

In one embodiment, the N-glycan on the N-glycosylated IL-2 domain in a fusion protein provided herein is oligomannose-type. In another embodiment, the N-glycan on the N-glycosylated IL-2 domain in a fusion protein provided herein is complex-type. In another embodiment, the N-glycan on the N-glycosylated IL-2 domain in a fusion protein provided herein is hydride-type.

In one embodiment, the N-glycan on the N-glycosylated IL-2 domain in a fusion protein provided herein is biantennary complex-type. In another embodiment, the N-glycan on the N-glycosylated IL-2 domain in a fusion protein provided herein is triantennary complex-type. In yet another embodiment, the N-glycan on the N-glycosylated IL-2 domain in a fusion protein provided herein is tetraantennary complex-type.

In one embodiment, the N-glycan on the N-glycosylated IL-2 domain in a fusion protein provided herein is one of the glycans described in Szabo et al., J. Proteome. Res. 2018, 17, 1559-1574, the disclosure of which is incorporated herein by reference in its entirety.

In one embodiment, the IL-2 domain in a fusion protein provided herein comprises, as set forth in SEQ ID NO: 1, 2, 3, 4, or 5, amino acid substitutions at positions R38 and F42, and optionally an amino acid substitution at position E15, H16, D20, K32, K76, S87, N88, or I92. In another embodiment, the IL-2 domain in a fusion protein provided herein comprises, as set forth in SEQ ID NO: 1, 2, 3, 4, or 5, amino acid substitutions at positions K32, R38, and F42, and optionally an amino acid substitution at position E15, H16, D20, K76, S87, N88, or I92. In yet another embodiment, the IL-2 domain in a fusion protein provided herein comprises, as set forth in SEQ ID NO: 1, 2, 3, 4, or 5, amino acid substitutions at positions R38, F42, and K76, and optionally an amino acid substitution at position E15, H16, D20, K32, S87, N88, or I92. In yet another embodiment, the IL-2 domain in a fusion protein provided herein comprises, as set forth in SEQ ID NO: 1, 2, 3, 4, or 5, amino acid substitutions at positions E15, K32, R38, and F42, and optionally an amino acid substitution at position H16, D20, K76, S87, N88, or I92. In still another embodiment, the IL-2 domain in a fusion protein provided herein comprises, as set forth in SEQ ID NO: 1, 2, 3, 4, or 5, amino acid substitutions at positions E15, R38, F42, and K76, and optionally an amino acid substitution at position H16, D20, K32, S87, N88, or I92.

In one embodiment, the IL-2 domain in a fusion protein provided herein comprises, as set forth in SEQ ID NO: 1, 2, 3, 4, or 5, amino acid substitutions of R38E and F42A or F42K, and optionally an amino acid substitution of E15K, H16E, H16F, H16I, H16V, D20A, D20E, D20K, D20T, K32E, K76E, S87D, N88A, I92A, I92D, I92E, or I92G. In another embodiment, the IL-2 domain in a fusion protein provided herein comprises, as set forth in SEQ ID NO: 1, 2, 3, 4, or 5, amino acid substitutions of K32E, R38E, and F42A or F42K, and optionally an amino acid substitution of E15K, H16E, H16F, H16I, H16V, D20A, D20E, D20K, D20T, K76E, S87D, N88A, I92A, I92D, I92E, or I92G. In yet another embodiment, the IL-2 domain in a fusion protein provided herein comprises, as set forth in SEQ ID NO: 1, 2, 3, 4, or 5, amino acid substitutions of R38E, F42A or F42K, and K76E, and optionally an amino acid substitution of E15K, H16E, H16F, H16I, H16V, D20A, D20E, D20K, D20T, K32E, S87D, N88A, I92A, I92D, I92E, or I92G. In yet another embodiment, the IL-2 domain in a fusion protein provided herein comprises, as set forth in SEQ ID NO: 1, 2, 3, 4, or 5, amino acid substitutions of E15K, K32E, R38E, and F42A or F42K, and optionally an amino acid substitution of H16E, H16F, H16I, H16V, D20A, D20E, D20K, D20T, K76E, S87D, N88A, I92A, I92D, I92E, or I92G. In still another embodiment, the IL-2 domain in a fusion protein provided herein comprises, as set forth in SEQ ID NO: 1, 2, 3, 4, or 5, amino acid substitutions of E15K, R38E, F42A or F42K, and K76E, and optionally an amino acid substitution of H16E, H16F, H16I, H16V, D20A, D20E, D20K, D20T, K32E, S87D, N88A, I92A, I92D, I92E, or I92G.

In one embodiment, the interleukin-2 domain in a fusion protein provided herein comprises amino acid substitutions at positions K32, R38, and F42 as set forth in SEQ ID NO: 1, 2, 3, 4, or 5. In another embodiment, the interleukin-2 domain in a fusion protein provided herein comprises amino acid substitutions of K32E, R38E, and F42A or F42K as set forth in SEQ ID NO: 1, 2, 3, 4, or 5. In yet another embodiment, the interleukin-2 domain in a fusion protein provided herein comprises amino acid substitutions of K32E, R38E, and F42A as set forth in SEQ ID NO: 1, 2, 3, 4, or 5. In still another embodiment, the interleukin-2 domain in a fusion protein provided herein comprises amino acid substitutions of K32E, R38E, and F42K as set forth in SEQ ID NO: 1, 2, 3, 4, or 5.

In one embodiment, the interleukin-2 domain in a fusion protein provided herein comprises amino acid substitutions at positions R38, F42, and K76 as set forth in SEQ ID NO: 1, 2, 3, 4, or 5. In another embodiment, the interleukin-2 domain in a fusion protein provided herein comprises amino acid substitutions of R38E, F42A or F42K, and K76E as set forth in SEQ ID NO: 1, 2, 3, 4, or 5. In yet another embodiment, the interleukin-2 domain in a fusion protein provided herein comprises amino acid substitutions of R38E, F42A, and K76E as set forth in SEQ ID NO: 1, 2, 3, 4, or 5. In still another embodiment, the interleukin-2 domain in a fusion protein provided herein comprises amino acid substitutions of R38E, F42K, and K76E as set forth in SEQ ID NO: 1, 2, 3, 4, or 5.

In one embodiment, the interleukin-2 domain in a fusion protein provided herein comprises amino acid substitutions at positions E15, K32, R38, and F42 as set forth in SEQ ID NO: 1, 2, 3, 4, or 5. In another embodiment, the interleukin-2 domain in a fusion protein provided herein comprises amino acid substitutions of E15K, K32E, R38E, and F42A or F42K as set forth in SEQ ID NO: 1, 2, 3, 4, or 5. In yet another embodiment, the interleukin-2 domain in a fusion protein provided herein comprises amino acid substitutions of E15K, K32E, R38E, and F42A as set forth in SEQ ID NO: 1, 2, 3, 4, or 5. In still another embodiment, the interleukin-2 domain in a fusion protein provided herein comprises amino acid substitutions of E15K, K32E, R38E, and F42K as set forth in SEQ ID NO: 1, 2, 3, 4, or 5.

In one embodiment, the interleukin-2 domain in a fusion protein provided herein comprises amino acid substitutions at positions E15, R38, F42, and K76 as set forth in SEQ ID NO: 1, 2, 3, 4, or 5. In another embodiment, the interleukin-2 domain in a fusion protein provided herein comprises amino acid substitutions of E15K, R38E, F42A or F42K, and K76E as set forth in SEQ ID NO: 1, 2, 3, 4, or 5. In yet another embodiment, the interleukin-2 domain in a fusion protein provided herein comprises amino acid substitutions of E15K, R38E, F42A, and K76E as set forth in SEQ ID NO: 1, 2, 3, 4, or 5. In still another embodiment, the interleukin-2 domain in a fusion protein provided herein comprises amino acid substitutions of E15K, R38E, F42K, and K76E as set forth in SEQ ID NO: 1, 2, 3, 4, or 5.

In one embodiment, the IL-2 domain in a fusion protein provided herein comprises an amino acid sequence of any one of SEQ ID NOs: 14 to 25.

In one embodiment, the interleukin-2 domain in a fusion protein provided herein comprises, set forth in SEQ ID NO: 1, 2, 3, 4, or 5, a disulfide bond formed between two different amino acid residues from positions N30 to L80; and optionally an amino acid substitution at position E15, H16, D20, K32, K76, S87, N88, or I92. In another embodiment, the interleukin-2 domain in a fusion protein provided herein comprises, set forth in SEQ ID NO: 1, 2, 3, 4, or 5, a disulfide bond formed between two different amino acid residues from positions K35 to L72; and optionally an amino acid substitution at position E15, H16, D20, K32, K76, S87, N88, or I92. In yet another embodiment, the interleukin-2 domain in a fusion protein provided herein comprises, set forth in SEQ ID NO: 1, 2, 3, 4, or 5, a disulfide bond formed between two different amino acid residues from positions K35 to L69; and optionally an amino acid substitution at position E15, H16, D20, K32, K76, S87, N88, or I92.

In one embodiment, the interleukin-2 domain in a fusion protein provided herein comprises, set forth in SEQ ID NO: 1, 2, 3, 4, or 5, a disulfide bond formed between two different amino acid residues from positions N30 to L80; and optionally an amino acid substitution of E15K, H16E, H16F, H16I, H16V, D20A, D20E, D20K, D20T, K32E, K76E, S87D, N88A, I92A, I92D, I92E, or I92G. In another embodiment, the interleukin-2 domain in a fusion protein provided herein comprises, set forth in SEQ ID NO: 1, 2, 3, 4, or 5, a disulfide bond formed between two different amino acid residues from positions K35 to L72; and optionally an amino acid substitution of E15K, H16E, H16F, H16I, H16V, D20A, D20E, D20K, D20T, K32E, K76E, S87D, N88A, I92A, I92D, I92E, or I92G. In yet another embodiment, the interleukin-2 domain in a fusion protein provided herein comprises, set forth in SEQ ID NO: 1, 2, 3, 4, or 5, a disulfide bond formed between two different amino acid residues from positions K35 to L69; and optionally an amino acid substitution of E15K, H16E, H16F, H16I, H16V, D20A, D20E, D20K, D20T, K32E, K76E, S87D, N88A, I92A, I92D, I92E, or I92G.

In one embodiment, the interleukin-2 domain in a fusion protein provided herein comprises a disulfide bond formed between two different amino acid residues from positions N30 to L80 as set forth in SEQ ID NO: 1, 2, 3, 4, or 5. In another embodiment, the interleukin-2 domain in a fusion protein provided herein comprises a disulfide bond formed between two different amino acid residues from positions K35 to L72 as set forth in SEQ ID NO: 1, 2, 3, 4, or 5. In yet another embodiment, the interleukin-2 domain in a fusion protein provided herein comprises a disulfide bond formed between two different amino acid residues from positions K35 to L69 as set forth in SEQ ID NO: 1, 2, 3, 4, or 5.

In one embodiment, the interleukin-2 domain in a fusion protein provided herein comprises a disulfide bond formed between two different amino acid residues, each independently at K35, R38, F42, Y45, E62, V69, or L72 as set forth in SEQ ID NO: 1, 2, 3, 4, or 5. In another embodiment, the interleukin-2 domain in a fusion protein provided herein comprises a disulfide bond formed between an amino acid residue at position K35, R38, F42, or Y45; and an amino acid residue at position E62, V69, or L72 as set forth in SEQ ID NO: 1, 2, 3, 4, or 5. In yet another embodiment, the interleukin-2 domain in a fusion protein provided herein comprises a disulfide bond formed between an amino acid residue at position F42 and an amino acid residue at position E62, V69, or L72 as set forth in SEQ ID NO: 1, 2, 3, 4, or 5. In still another embodiment, the interleukin-2 domain in a fusion protein provided herein comprises a disulfide bond formed between an amino acid residue at position K35, R38, F42, or Y45, and an amino acid residue at position V69 as set forth in SEQ ID NO: 1, 2, 3, 4, or 5.

In one embodiment, the interleukin-2 domain in a fusion protein provided herein comprises a disulfide bond formed between K35C and L72C, R38C and L72C, F42C and V69C, Y42C and L72C, or Y45C and E62C. In another embodiment, the interleukin-2 domain in the fusion protein comprises a disulfide bond formed between K35C and L72C. In yet another embodiment, the interleukin-2 domain in the fusion protein comprises a disulfide bond formed between R38C and L72C. In yet another embodiment, the interleukin-2 domain in the fusion protein comprises a disulfide bond formed between F42C and V69C. In yet another embodiment, the interleukin-2 domain in the fusion protein comprises a disulfide bond formed between Y42C and L72C. In still another embodiment, the interleukin-2 domain in the fusion protein comprises a disulfide bond formed between Y45C and E62C.

In one embodiment, the interleukin-2 domain in a fusion protein provided herein comprises, as set forth in SEQ ID NO: 1, 2, 3, 4, or 5, (i) an amino acid substitution at position K8, K9, Q13, E15, H16, L19, D20, M23, K32, P34, K35, L36, T37, R38, M39, L40, T41, F42, K43, F44, Y45, M46, P47, E61, E62, L63, K64, P65, L66, E67, E68, V69, L70, N71, L72, A73, Q74, K76, H79, R81, D84, S87, N88, V91, I92, E95, Y107, D109, T111, S127, or S130; and (ii) a disulfide bond formed between two different amino acid residues from positions N30 to L80.

In another embodiment, the interleukin-2 domain in a fusion protein provided herein comprises, as set forth in SEQ ID NO: 1, 2, 3, 4, or 5, (i) an amino acid substitution of K8D, K8E, K8Q, K9D, K9E, K9Q, Q13E, Q13N, E15K, E15Q, E15V, H16E, H16F, H16I, H16V, L19S, D20A, D20E, D20K, D20T, M23K, K32D, K32E, K32Q, P34N, K35N, L36S, L36T, T37N, R38E, R38N, M39N, L40S, L40T, T41N, F42A, F42C, F42K, F42N, K43N, F44N, Y45N, M46S, M46T, P47S, P47T, E61N, E62N, L63S, L63T, K64N, P65N, L66N, E67S, E67T, E68N, V69N, L70S, L70T, N71S, N71T, L72N, A73S, A73T, Q74S, Q74T, K76D, K76E, K76Q, H79D, H79E, H79Q, R81D, R81E, R81Q, D84T, S87D, S87E, N88A, V91I, I92A, I92D, I92E, I92G, E95K, E95N, E95Q, Y107N, D109N, T111S, S127A, S127E, S127F, S127W, S130A, S130E, S130F, or; and (ii) a disulfide bond formed between two different amino acid residues, each independently at position K35, R38, F42, Y45, E62, V69, or L72.

In yet another embodiment, the interleukin-2 domain in a fusion protein provided herein comprises, as set forth in SEQ ID NO: 1, 2, 3, 4, or 5, (i) an amino acid substitution of E15K, H16E, H16F, H16I, H16V, D20A, D20E, D20K, D20T, K32E, K76E, S87D, N88A, I92A, I92D, I92E, or I92G; and (ii) a disulfide bond formed between F42C and V69C.

In yet another embodiment, the interleukin-2 domain in a fusion protein provided herein comprises, as set forth in SEQ ID NO: 1, 2, 3, 4, or 5, (i) an amino acid substitution of E15K, H16E, H16I, H16V, D20A, D20E, D20K, D20T, K32E, K76E, S87D, N88A, I92A, I92D, I92E, or I92G; and (ii) a disulfide bond formed between F42C and V69C.

In still another embodiment, the interleukin-2 domain in a fusion protein provided herein comprises, as set forth in SEQ ID NO: 1, 2, 3, 4, or 5, (i) an amino acid substitution of E15K, K32E, or K76E; and (ii) a disulfide bond formed between F42C and V69C.

In one embodiment, the interleukin-2 domain in a fusion protein provided herein comprises a disulfide bond formed between F42C and V69C as set forth in SEQ ID NO: 1, 2, 3, 4, or 5. In another embodiment, the interleukin-2 domain in a fusion protein provided herein comprises, as set forth in SEQ ID NO: 1, 2, 3, 4, or 5, a disulfide bond formed between F42C and V69C, an amino acid substitution at position K32 or K76, and optionally an amino acid substitution at position E15. In yet another embodiment, the interleukin-2 domain in a fusion protein provided herein comprises, as set forth in SEQ ID NO: 1, 2, 3, 4, or 5, a disulfide bond formed between F42C and V69C, and an amino acid substitution of K32E. In yet another embodiment, the interleukin-2 domain in a fusion protein provided herein comprises, as set forth in SEQ ID NO: 1, 2, 3, 4, or 5, a disulfide bond formed between F42C and V69C, and an amino acid substitution of K76E. In yet another embodiment, the interleukin-2 domain in a fusion protein provided herein comprises, as set forth in SEQ ID NO: 1, 2, 3, 4, or 5, a disulfide bond formed between F42C and V69C, and amino acid substitutions of E15K and K32E. In still another embodiment, the interleukin-2 domain in a fusion protein provided herein comprises, as set forth in SEQ ID NO: 1, 2, 3, 4, or 5, a disulfide bond formed between F42C and V69C, and amino acid substitutions of E15K and K76E.

In one embodiment, the IL-2 domain in a fusion protein provided herein comprises an amino acid sequence of any one of SEQ ID NOs: 26 to 37.

In one embodiment, the interleukin-2 domain in a fusion protein provided herein comprises, as set forth in SEQ ID NO: 1, 2, 3, 4, or 5, a replacement of the amino acid residues from positions N29 to A50 with an IL-15 hinge fragment-containing peptide, and optionally an amino acid substitution at position E15, H16, D20, K32, K76, S87, N88, or I92. In another embodiment, the interleukin-2 domain in a fusion protein provided herein comprises, as set forth in SEQ ID NO: 1, 2, 3, 4, or 5, a replacement of the amino acid residues from positions N29 to L40 with an IL-15 hinge fragment-containing peptide, and optionally an amino acid substitution at position E15, H16, D20, K32, K76, S87, N88, or I92. In yet another embodiment, the interleukin-2 domain in a fusion protein provided herein comprises, as set forth in SEQ ID NO: 1, 2, 3, 4, or 5, a replacement of the amino acid residues from positions N29 to L40 having an amino acid sequence of SEQ ID NO: 39 with an IL-15 hinge fragment-containing peptide having an amino acid sequence of SEQ ID NO: 40 or 41, and optionally an amino acid substitution at position E15, H16, D20, K32, K76, S87, N88, or I92.

In one embodiment, the interleukin-2 domain in a fusion protein provided herein comprises, as set forth in SEQ ID NO: 1, 2, 3, 4, or 5, a replacement of the amino acid residues from positions N29 to A50 with an IL-15 hinge fragment-containing peptide, and optionally an amino acid substitution of E15K, H16E, H16F, H16I, H16V, D20A, D20E, D20K, D20T, K32E, K76E, S87D, N88A, I92A, I92D, I92E, or I92G. In another embodiment, the interleukin-2 domain in a fusion protein provided herein comprises, as set forth in SEQ ID NO: 1, 2, 3, 4, or 5, a replacement of the amino acid residues from positions N29 to L40 with an IL-15 hinge fragment-containing peptide, and optionally an amino acid substitution of E15K, H16E, H16F, H16I, H16V, D20A, D20E, D20K, D20T, K32E, K76E, S87D, N88A, I92A, I92D, I92E, or I92G. In yet another embodiment, the interleukin-2 domain in a fusion protein provided herein comprises, as set forth in SEQ ID NO: 1, 2, 3, 4, or 5, a replacement of the amino acid residues from positions N29 to L40 having an amino acid sequence of SEQ ID NO: 39 with an IL-15 hinge fragment-containing peptide having an amino acid sequence of SEQ ID NO: 40 or 41, and optionally an amino acid substitution of E15K, H16E, H16F, H16I, H16V, D20A, D20E, D20K, D20T, K32E, K76E, S87D, N88A, I92A, I92D, I92E, or I92G.

In one embodiment, the IL-15 hinge fragment-containing peptide has an amino acid sequence of SEQ ID NO: 40 or 41. In another embodiment, the IL-15 hinge fragment-containing peptide has an amino acid sequence of SEQ ID NO: 40. In yet another embodiment, the IL-15 hinge fragment-containing peptide has an amino acid sequence of SEQ ID NO: 41.

In one embodiment, the interleukin-2 domain in a fusion protein provided herein comprises a replacement of the amino acid residues from positions N29 to A50 as set forth in SEQ ID NO: 1, 2, 3, 4, or 5 with an IL-15 hinge fragment-containing peptide. In another embodiment, the interleukin-2 domain in a fusion protein provided herein comprises a replacement of the amino acid residues from positions N29 to L40 as set forth in SEQ ID NO: 1, 2, 3, 4, or 5 with an IL-15 hinge fragment-containing peptide. In yet another embodiment, the interleukin-2 domain in a fusion protein provided herein comprises a replacement of the amino acid residues from positions N29 to L40 having an amino acid sequence of SEQ ID NO: 39 as set forth in SEQ ID NO: 1, 2, 3, 4, or 5 with an IL-15 hinge fragment-containing peptide having an amino acid sequence of SEQ ID NO: 40 or 41.

In one embodiment, the interleukin-2 domain in a fusion protein provided herein comprises, as set forth in SEQ ID NO: 1, 2, 3, 4, or 5, (i) an amino acid substitution at position K8, K9, Q13, E15, H16, L19, D20, M23, E61, E62, L63, K64, P65, L66, E67, E68, V69, L70, N71, L72, A73, Q74, K76, H79, R81, D84, S87, N88, V91, I92, E95, Y107, D109, T111, S127, or S130; and (ii) a replacement of the amino acid residues from positions N29 to A50 with an IL-15 hinge fragment-containing peptide.

In another embodiment, the interleukin-2 domain in a fusion protein provided herein comprises, as set forth in SEQ ID NO: 1, 2, 3, 4, or 5, (i) an amino acid substitution of K8D, K8E, K8Q, K9D, K9E, K9Q, Q13E, Q13N, E15K, E15Q, E15V, H16D, H16E, H16F, H16I, H16N, H16Q, H16V, L19S, D20A, D20E, D20K, D20T, M23K, E61N, E62N, L63S, L63T, K64N, P65N, L66N, E67S, E67T, E68N, V69N, L70S, L70T, N71S, N71T, L72N, A73S, A73T, Q74S, Q74T, K76D, K76E, K76Q, H79D, H79E, H79Q, R81D, R81E, R81Q, D84T, S87D, S87E, N88A, V91I, I92A, I92D, I92E, I92G, I92L, E95K, E95N, E95Q, Y107N, D109N, T111S, S127A, S127E, S127F, S127W, S130A, S130E, S130F, or S130W; and (ii) a replacement of the amino acid residues from positions N29 to A50 having an amino acid sequence of SEQ ID NO: 38 with an IL-15 hinge fragment-containing peptide.

In yet another embodiment, the interleukin-2 domain in a fusion protein provided herein comprises, as set forth in SEQ ID NO: 1, 2, 3, 4, or 5, (i) an amino acid substitution of K8D, K8E, K8Q, K9D, K9E, K9Q, Q13E, Q13N, E15K, E15Q, E15V, H16D, H16E, H16F, H16I, H16N, H16Q, H16V, L19S, D20A, D20E, D20K, D20T, M23K, E61N, E62N, L63S, L63T, K64N, P65N, L66N, E67S, E67T, E68N, V69N, L70S, L70T, N71S, N71T, L72N, A73S, A73T, Q74S, Q74T, K76D, K76E, K76Q, H79D, H79E, H79Q, R81D, R81E, R81Q, D84T, S87D, S87E, N88A, V91I, I92A, I92D, I92E, I92G, I92L, E95K, E95N, E95Q, Y107N, D109N, T111S, S127A, S127E, S127F, S127W, S130A, S130E, S130F, or S130W; and (ii) a replacement of the amino acid residues from positions N29 to L40 having an amino acid sequence of SEQ ID NO: 39 with an IL-15 hinge fragment-containing peptide.

In one embodiment, the interleukin-2 domain in a fusion protein provided herein comprises, as set forth in SEQ ID NO: 1, 2, 3, 4, or 5, (i) an amino acid substitution at position E15, H16, D20, S87, N88, or I92; and (ii) a replacement of the amino acid residues from positions N29 to A50 with an IL-15 hinge fragment-containing peptide.

In another embodiment, the interleukin-2 domain in a fusion protein provided herein comprises, as set forth in SEQ ID NO: 1, 2, 3, 4, or 5, (i) an amino acid substitution of E15K, H16E, H16F, H16I, H16V, D20A, D20E, D20K, D20T, S87D, N88A, I92A, I92D, I92E, or I92G; and (ii) a replacement of the amino acid residues from positions N29 to A50 having an amino acid sequence of SEQ ID NO: 38 with an IL-15 hinge fragment-containing peptide.

In yet another embodiment, the interleukin-2 domain in a fusion protein provided herein comprises, as set forth in SEQ ID NO: 1, 2, 3, 4, or 5, (i) an amino acid substitution of E15K, H16E, H16F, H16I, H16V, D20A, D20E, D20K, D20T, S87D, N88A, I92A, I92D, I92E, or I92G; and (ii) a replacement of the amino acid residues from positions N29 to L40 having an amino acid sequence of SEQ ID NO: 39 with an IL-15 hinge fragment-containing peptide.

In yet another embodiment, the interleukin-2 domain in a fusion protein provided herein comprises, as set forth in SEQ ID NO: 1, 2, 3, 4, or 5, (i) an amino acid substitution of E15K, H16E, H16F, H16I, H16V, D20A, D20E, D20K, D20T, S87D, N88A, I92A, I92D, I92E, or I92G; and (ii) a replacement of the amino acid residues from positions N29 to L40 having an amino acid sequence of SEQ ID NO: 39 with an IL-15 hinge fragment-containing peptide.

In still another embodiment, the interleukin-2 domain in a fusion protein provided herein comprises, as set forth in SEQ ID NO: 1, 2, 3, 4, or 5, (i) an amino acid substitution of E15K, H16E, H16F, H16I, H16V, D20A, D20E, D20K, D20T, S87D, N88A, I92A, I92D, I92E, or I92G; and (ii) a replacement of the amino acid residues from positions N29 to L40 having an amino acid sequence of SEQ ID NO: 39 with an IL-15 hinge fragment-containing peptide having an amino acid sequence of SEQ ID NO: 40 or 41.

In one embodiment, the interleukin-2 domain in a fusion protein provided herein comprises, as set forth in SEQ ID NO: 1, 2, 3, 4, or 5, (i) an amino acid substitution at E15; and (ii) a replacement of the amino acid residues from positions N29 to L40 having an amino acid sequence of SEQ ID NO: 39 with an IL-15 hinge fragment-containing peptide having an amino acid sequence of SEQ ID NO: 40 or 41. In another embodiment, the interleukin-2 domain in a fusion protein provided herein comprises, as set forth in SEQ ID NO: 1, 2, 3, 4, or 5, (i) an amino acid substitution of E15K; and (ii) a replacement of the amino acid residues from positions N29 to L40 having an amino acid sequence of SEQ ID NO: 39 with an IL-15 hinge fragment-containing peptide having an amino acid sequence of SEQ ID NO: 40 or 41.

In one embodiment, the interleukin-2 domain in a fusion protein provided herein comprises, as set forth in SEQ ID NO: 1, 2, 3, 4, or 5, (i) an amino acid substitution of H16; and (ii) a replacement of the amino acid residues from positions N29 to L40 having an amino acid sequence of SEQ ID NO: 39 with an IL-15 hinge fragment-containing peptide having an amino acid sequence of SEQ ID NO: 40 or 41. In another embodiment, the interleukin-2 domain in a fusion protein provided herein comprises, as set forth in SEQ ID NO: 1, 2, 3, 4, or 5, (i) an amino acid substitution of H16E, H16F, H16I, or H16V; and (ii) a replacement of the amino acid residues from positions N29 to L40 having an amino acid sequence of SEQ ID NO: 39 with an IL-15 hinge fragment-containing peptide having an amino acid sequence of SEQ ID NO: 40 or 41. In yet another embodiment, the interleukin-2 domain in a fusion protein provided herein comprises, as set forth in SEQ ID NO: 1, 2, 3, 4, or 5, (i) an amino acid substitution of H16E, H16I, or H16V; and (ii) a replacement of the amino acid residues from positions N29 to L40 having an amino acid sequence of SEQ ID NO: 39 with an IL-15 hinge fragment-containing peptide having an amino acid sequence of SEQ ID NO: 40 or 41. In yet another embodiment, the interleukin-2 domain in a fusion protein provided herein comprises, as set forth in SEQ ID NO: 1, 2, 3, 4, or 5, (i) an amino acid substitution of H16E; and (ii) a replacement of the amino acid residues from positions N29 to L40 having an amino acid sequence of SEQ ID NO: 39 with an IL-15 hinge fragment-containing peptide having an amino acid sequence of SEQ ID NO: 40 or 41. In yet another embodiment, the interleukin-2 domain in a fusion protein provided herein comprises, as set forth in SEQ ID NO: 1, 2, 3, 4, or 5, (i) an amino acid substitution of H16F; and (ii) a replacement of the amino acid residues from positions N29 to L40 having an amino acid sequence of SEQ ID NO: 39 with an IL-15 hinge fragment-containing peptide having an amino acid sequence of SEQ ID NO: 40 or 41. In yet another embodiment, the interleukin-2 domain in a fusion protein provided herein comprises, as set forth in SEQ ID NO: 1, 2, 3, 4, or 5, (i) an amino acid substitution of H16I; and (ii) a replacement of the amino acid residues from positions N29 to L40 having an amino acid sequence of SEQ ID NO: 39 with an IL-15 hinge fragment-containing peptide having an amino acid sequence of SEQ ID NO: 40 or 41. In still another embodiment, the interleukin-2 domain in a fusion protein provided herein comprises, as set forth in SEQ ID NO: 1, 2, 3, 4, or 5, (i) an amino acid substitution of H16V; and (ii) a replacement of the amino acid residues from positions N29 to L40 having an amino acid sequence of SEQ ID NO: 39 with an IL-15 hinge fragment-containing peptide having an amino acid sequence of SEQ ID NO: 40 or 41.

In one embodiment, the interleukin-2 domain in a fusion protein provided herein comprises, as set forth in SEQ ID NO: 1, 2, 3, 4, or 5, (i) an amino acid substitution of D20; and (ii) a replacement of the amino acid residues from positions N29 to L40 having an amino acid sequence of SEQ ID NO: 39 with an IL-15 hinge fragment-containing peptide having an amino acid sequence of SEQ ID NO: 40 or 41. In another embodiment, the interleukin-2 domain in a fusion protein provided herein comprises, as set forth in SEQ ID NO: 1, 2, 3, 4, or 5, (i) an amino acid substitution of D20A, D20E, D20K, or D20T; and (ii) a replacement of the amino acid residues from positions N29 to L40 having an amino acid sequence of SEQ ID NO: 39 with an IL-15 hinge fragment-containing peptide having an amino acid sequence of SEQ ID NO: 40 or 41. In yet another embodiment, the interleukin-2 domain in a fusion protein provided herein comprises, as set forth in SEQ ID NO: 1, 2, 3, 4, or 5, (i) an amino acid substitution of D20A; and (ii) a replacement of the amino acid residues from positions N29 to L40 having an amino acid sequence of SEQ ID NO: 39 with an IL-15 hinge fragment-containing peptide having an amino acid sequence of SEQ ID NO: 40 or 41. In yet another embodiment, the interleukin-2 domain in a fusion protein provided herein comprises, as set forth in SEQ ID NO: 1, 2, 3, 4, or 5, (i) an amino acid substitution of D20E; and (ii) a replacement of the amino acid residues from positions N29 to L40 having an amino acid sequence of SEQ ID NO: 39 with an IL-15 hinge fragment-containing peptide having an amino acid sequence of SEQ ID NO: 40 or 41. In yet another embodiment, the interleukin-2 domain in a fusion protein provided herein comprises, as set forth in SEQ ID NO: 1, 2, 3, 4, or 5, (i) an amino acid substitution of D20K; and (ii) a replacement of the amino acid residues from positions N29 to L40 having an amino acid sequence of SEQ ID NO: 39 with an IL-15 hinge fragment-containing peptide having an amino acid sequence of SEQ ID NO: 40 or 41. In still another embodiment, the interleukin-2 domain in a fusion protein provided herein comprises, as set forth in SEQ ID NO: 1, 2, 3, 4, or 5, (i) an amino acid substitution of D20T; and (ii) a replacement of the amino acid residues from positions N29 to L40 having an amino acid sequence of SEQ ID NO: 39 with an IL-15 hinge fragment-containing peptide having an amino acid sequence of SEQ ID NO: 40 or 41.

In one embodiment, the interleukin-2 domain in a fusion protein provided herein comprises, as set forth in SEQ ID NO: 1, 2, 3, 4, or 5, (i) an amino acid substitution at S87; and (ii) a replacement of the amino acid residues from positions N29 to L40 having an amino acid sequence of SEQ ID NO: 39 with an IL-15 hinge fragment-containing peptide having an amino acid sequence of SEQ ID NO: 40 or 41. In another embodiment, the interleukin-2 domain in a fusion protein provided herein comprises, as set forth in SEQ ID NO: 1, 2, 3, 4, or 5, (i) an amino acid substitution of S87D; and (ii) a replacement of the amino acid residues from positions N29 to L40 having an amino acid sequence of SEQ ID NO: 39 with an IL-15 hinge fragment-containing peptide having an amino acid sequence of SEQ ID NO: 40 or 41.

In one embodiment, the interleukin-2 domain in a fusion protein provided herein comprises, as set forth in SEQ ID NO: 1, 2, 3, 4, or 5, (i) an amino acid substitution at N88; and (ii) a replacement of the amino acid residues from positions N29 to L40 having an amino acid sequence of SEQ ID NO: 39 with an IL-15 hinge fragment-containing peptide having an amino acid sequence of SEQ ID NO: 40 or 41. In another embodiment, the interleukin-2 domain in a fusion protein provided herein comprises, as set forth in SEQ ID NO: 1, 2, 3, 4, or 5, (i) an amino acid substitution of N88A; and (ii) a replacement of the amino acid residues from positions N29 to L40 having an amino acid sequence of SEQ ID NO: 39 with an IL-15 hinge fragment-containing peptide having an amino acid sequence of SEQ ID NO: 40 or 41.

In one embodiment, the interleukin-2 domain in a fusion protein provided herein comprises, as set forth in SEQ ID NO: 1, 2, 3, 4, or 5, (i) an amino acid substitution at I92; and (ii) a replacement of the amino acid residues from positions N29 to L40 having an amino acid sequence of SEQ ID NO: 39 with an IL-15 hinge fragment-containing peptide having an amino acid sequence of SEQ ID NO: 40 or 41. In another embodiment, the interleukin-2 domain in a fusion protein provided herein comprises, as set forth in SEQ ID NO: 1, 2, 3, 4, or 5, (i) an amino acid substitution of I92A, I92D, I92E, or I92G; and (ii) a replacement of the amino acid residues from positions N29 to L40 having an amino acid sequence of SEQ ID NO: 39 with an IL-15 hinge fragment-containing peptide having an amino acid sequence of SEQ ID NO: 40 or 41. In yet another embodiment, the interleukin-2 domain in a fusion protein provided herein comprises, as set forth in SEQ ID NO: 1, 2, 3, 4, or 5, (i) an amino acid substitution of I92A; and (ii) a replacement of the amino acid residues from positions N29 to L40 having an amino acid sequence of SEQ ID NO: 39 with an IL-15 hinge fragment-containing peptide having an amino acid sequence of SEQ ID NO: 40 or 41. In yet another embodiment, the interleukin-2 domain in a fusion protein provided herein comprises, as set forth in SEQ ID NO: 1, 2, 3, 4, or 5, (i) an amino acid substitution of I92D; and (ii) a replacement of the amino acid residues from positions N29 to L40 having an amino acid sequence of SEQ ID NO: 39 with an IL-15 hinge fragment-containing peptide having an amino acid sequence of SEQ ID NO: 40 or 41. In yet another embodiment, the interleukin-2 domain in a fusion protein provided herein comprises, as set forth in SEQ ID NO: 1, 2, 3, 4, or 5, (i) an amino acid substitution of I92E; and (ii) a replacement of the amino acid residues from positions N29 to L40 having an amino acid sequence of SEQ ID NO: 39 with an IL-15 hinge fragment-containing peptide having an amino acid sequence of SEQ ID NO: 40 or 41. In still another embodiment, the interleukin-2 domain in a fusion protein provided herein comprises, as set forth in SEQ ID NO: 1, 2, 3, 4, or 5, (i) an amino acid substitution of I92G; and (ii) a replacement of the amino acid residues from positions N29 to L40 having an amino acid sequence of SEQ ID NO: 39 with an IL-15 hinge fragment-containing peptide having an amino acid sequence of SEQ ID NO: 40 or 41.

In one embodiment, the interleukin-2 domain in a fusion protein provided herein comprises, as set forth in SEQ ID NO: 1, 2, 3, 4, or 5, (i) one or two amino acid substitutions of E15K, H16I, D20T, N88A, and I92A; and (ii) a replacement of the amino acid residues from positions N29 to L40 having an amino acid sequence of SEQ ID NO: 39 with an IL-15 hinge fragment-containing peptide having an amino acid sequence of SEQ ID NO: 40 or 41. In another embodiment, the interleukin-2 domain in a fusion protein provided herein comprises, as set forth in SEQ ID NO: 1, 2, 3, 4, or 5, (i) an amino acid substitution of E15K, H16I, D20T, N88A, or I92A; and (ii) a replacement of the amino acid residues from positions N29 to L40 having an amino acid sequence of SEQ ID NO: 39 with an IL-15 hinge fragment-containing peptide having an amino acid sequence of SEQ ID NO: 40 or 41. In yet another embodiment, the interleukin-2 domain in a fusion protein provided herein comprises, as set forth in SEQ ID NO: 1, 2, 3, 4, or 5, (i) an amino acid substitution of E15K; (ii) an amino acid substitution of H16I, D20T, N88A, or I92A; and (iii) a replacement of the amino acid residues from positions N29 to L40 having an amino acid sequence of SEQ ID NO: 39 with an IL-15 hinge fragment-containing peptide having an amino acid sequence of SEQ ID NO: 40 or 41.

In one embodiment, the IL-2 domain in a fusion protein provided herein comprises an amino acid sequence of any one of SEQ ID NOs: 42 to 81, 255, and 256. In another embodiment, the IL-2 domain in a fusion protein provided herein comprises an amino acid sequence of SEQ ID NO: 56, 60, 68, 70, 80, 255, or 256. In yet another embodiment, the IL-2 domain in a fusion protein provided herein comprises an amino acid sequence of SEQ ID NO: 56. In yet another embodiment, the IL-2 domain in a fusion protein provided herein comprises an amino acid sequence of SEQ ID NO: 60. In yet another embodiment, the IL-2 domain in a fusion protein provided herein comprises an amino acid sequence of SEQ ID NO: 68. In yet another embodiment, the IL-2 domain in a fusion protein provided herein comprises an amino acid sequence of SEQ ID NO: 70. In yet another embodiment, the IL-2 domain in a fusion protein provided herein comprises an amino acid sequence of SEQ ID NO: 80. In yet another embodiment, the IL-2 domain in a fusion protein provided herein comprises an amino acid sequence of SEQ ID NO: 255. In still another embodiment, the IL-2 domain in a fusion protein provided herein comprises an amino acid sequence of SEQ ID NO: 256.

In certain embodiments, the interleukin-2 domain in a fusion protein provided herein comprises an amino acid sequence that is no less than about 80%, no less than about 85%, no less than about 90%, no less than about 91%, no less than about 92%, no less than about 93%, no less than about 94%, no less than about 95%, no less than about 96%, no less than about 97%, no less than about 98%, or no less than about 99% identical to the amino acid sequence of SEQ ID NO: 1, 2, 3, 4, or 5. In certain embodiments, the interleukin-2 domain in a fusion protein provided herein comprises an amino acid sequence that is no less than about 80% identical to the amino acid sequence of SEQ ID NO: 1, 2, 3, 4, or 5. In certain embodiments, the interleukin-2 domain in a fusion protein provided herein comprises an amino acid sequence that is no less than about 85% identical to the amino acid sequence of SEQ ID NO: 1, 2, 3, 4, or 5. In certain embodiments, the interleukin-2 domain in a fusion protein provided herein comprises an amino acid sequence that is no less than about 90% identical to the amino acid sequence of SEQ ID NO: 1, 2, 3, 4, or 5. In certain embodiments, the interleukin-2 domain in a fusion protein provided herein comprises an amino acid sequence that is no less than about 91% identical to the amino acid sequence of SEQ ID NO: 1, 2, 3, 4, or 5. In certain embodiments, the interleukin-2 domain in a fusion protein provided herein comprises an amino acid sequence that is no less than about 92% identical to the amino acid sequence of SEQ ID NO: 1, 2, 3, 4, or 5. In certain embodiments, the interleukin-2 domain in a fusion protein provided herein comprises an amino acid sequence that is no less than about 93% identical to the amino acid sequence of SEQ ID NO: 1, 2, 3, 4, or 5. In certain embodiments, the interleukin-2 domain in a fusion protein provided herein comprises an amino acid sequence that is no less than about 94% identical to the amino acid sequence of SEQ ID NO: 1, 2, 3, 4, or 5. In certain embodiments, the interleukin-2 domain in a fusion protein provided herein comprises an amino acid sequence that is no less than about 95% identical to the amino acid sequence of SEQ ID NO: 1, 2, 3, 4, or 5. In certain embodiments, the interleukin-2 domain in a fusion protein provided herein comprises an amino acid sequence that is no less than about 96% identical to the amino acid sequence of SEQ ID NO: 1, 2, 3, 4, or 5. In certain embodiments, the interleukin-2 domain in a fusion protein provided herein comprises an amino acid sequence that is no less than about 97% identical to the amino acid sequence of SEQ ID NO: 1, 2, 3, 4, or 5. In certain embodiments, the interleukin-2 domain in a fusion protein provided herein comprises an amino acid sequence that is no less than about 98% identical to the amino acid sequence of SEQ ID NO: 1, 2, 3, 4, or 5. In certain embodiments, the interleukin-2 domain in a fusion protein provided herein comprises an amino acid sequence that is no less than about 99% identical to the amino acid sequence of SEQ ID NO: 1, 2, 3, 4, or 5.

In certain embodiments, the interleukin-2 domain in a fusion protein provided herein comprises one of the amino acid sequences of interleukin-2 variants and muteins described in CN 111018961 A, US 2018/0326010 A1, and WO 2020/005819 A1, the disclosure of each of which is incorporated herein by reference in its entirety.

In certain embodiments, the interleukin-2 domain in a fusion protein provided herein further includes one or more additional substitutions, deletions, and/or insertions; and/or one or more additional post-translational modifications.

### PD-1 Binding Domain

In one embodiment, the PD-1 binding domain comprises:
(i) a light chain complementarity determining region 1 (CDRL1) of SEQ ID NO: 111,
   a light chain complementarity determining region 2 (CDRL2) of DAS,
   a light chain complementarity determining region 3 (CDRL3) of SEQ ID NO: 112,
   a heavy chain complementarity determining region 1 (CDRH1) of SEQ ID NO: 113,
   a heavy chain complementarity determining region 2 (CDRH2) of SEQ ID NO: 114, and
   a heavy chain complementarity determining region 3 (CDRH3) of SEQ ID NO: 115;
(ii) a CDRL1 of SEQ ID NO: 120, a CDRL2 of LAS, a CDRL3 of SEQ ID NO: 121, a CDRH1 of SEQ ID NO: 122, a CDRH2 of SEQ ID NO: 123, and a CDRH3 of SEQ ID NO: 124;
(iii) a CDRL1 of SEQ ID NO: 129, a CDRL2 of KVS, a CDRL3 of SEQ ID NO: 130, a CDRH1 of SEQ ID NO: 131, a CDRH2 of SEQ ID NO: 132, and a CDRH3 of SEQ ID NO: 133;
(iv) a CDRL1 of SEQ ID NO: 138, a CDRL2 of TAT, a CDRL3 of SEQ ID NO: 139, a CDRH1 of SEQ ID NO: 140, a CDRH2 of SEQ ID NO: 141, and a CDRH3 of SEQ ID NO: 142;
(v) a CDRL1 of SEQ ID NO: 147, a CDRL2 of AAS, a CDRL3 of SEQ ID NO: 148, a CDRH1 of SEQ ID NO: 149, a CDRH2 of SEQ ID NO: 150, and a CDRH3 of SEQ ID NO: 151;
(vi) a CDRL1 of SEQ ID NO: 156, a CDRL2 of WAS, a CDRL3 of SEQ ID NO: 157, a CDRH1 of SEQ ID NO: 158, a CDRH2 of SEQ ID NO: 159, and a CDRH3 of SEQ ID NO: 160;
(vii) a CDRL1 of SEQ ID NO: 165, a CDRL2 of YAF, a CDRL3 of SEQ ID NO: 166, a CDRH1 of SEQ ID NO: 167, a CDRH2 of SEQ ID NO: 168, and a CDRH3 of SEQ ID NO: 169;
(viii) a CDRL1 of SEQ ID NO: 174, a CDRL2 of AAS, a CDRL3 of SEQ ID NO: 175, a CDRH1 of SEQ ID NO: 176, a CDRH2 of SEQ ID NO: 177, and a CDRH3 of SEQ ID NO: 178;
(ix) a CDRL1 of SEQ ID NO: 183, a CDRL2 of WAS, a CDRL3 of SEQ ID NO: 184, a CDRH1 of SEQ ID NO: 185, a CDRH2 of SEQ ID NO: 186, and a CDRH3 of SEQ ID NO: 187; or
(x) a CDRL1 of SEQ ID NO: 192, a CDRL2 of AAS, a CDRL3 of SEQ ID NO: 193, a CDRH1 of SEQ ID NO: 194, a CDRH2 of SEQ ID NO: 195, and a CDRH3 of SEQ ID NO: 196.

In one embodiment, the PD-1 binding domain comprises a CDRL1 of SEQ ID NO: 111, a CDRL2 of DAS, a CDRL3 of SEQ ID NO: 112, a CDRH1 of SEQ ID NO: 113, a CDRH2 of SEQ ID NO: 114, and a CDRH3 of SEQ ID NO: 115.

In another embodiment, the PD-1 binding domain comprises a CDRL1 of SEQ ID NO: 120, a CDRL2 of LAS, a CDRL3 of SEQ ID NO: 121, a CDRH1 of SEQ ID NO: 122, a CDRH2 of SEQ ID NO: 123, and a CDRH3 of SEQ ID NO: 124.

In yet another embodiment, the PD-1 binding domain comprises a CDRL1 of SEQ ID NO: 129, a CDRL2 of KVS, a CDRL3 of SEQ ID NO: 130, a CDRH1 of SEQ ID NO: 131, a CDRH2 of SEQ ID NO: 132, and a CDRH3 of SEQ ID NO: 133.

In yet another embodiment, the PD-1 binding domain comprises a CDRL1 of SEQ ID NO: 138, a CDRL2 of TAT, a CDRL3 of SEQ ID NO: 139, a CDRH1 of SEQ ID NO: 140, a CDRH2 of SEQ ID NO: 141, and a CDRH3 of SEQ ID NO: 142.

In yet another embodiment, the PD-1 binding domain comprises a CDRL1 of SEQ ID NO: 147, a CDRL2 of AAS, a CDRL3 of SEQ ID NO: 148, a CDRH1 of SEQ ID NO: 149, a CDRH2 of SEQ ID NO: 150, and a CDRH3 of SEQ ID NO: 151.

In yet another embodiment, the PD-1 binding domain comprises a CDRL1 of SEQ ID NO: 156, a CDRL2 of WAS, a CDRL3 of SEQ ID NO: 157, a CDRH1 of SEQ ID NO: 158, a CDRH2 of SEQ ID NO: 159, and a CDRH3 of SEQ ID NO: 160.

In yet another embodiment, the PD-1 binding domain comprises a CDRL1 of SEQ ID NO: 165, a CDRL2 of YAF, a CDRL3 of SEQ ID NO: 166, a CDRH1 of SEQ ID NO: 167, a CDRH2 of SEQ ID NO: 168, and a CDRH3 of SEQ ID NO: 169.

In yet another embodiment, the PD-1 binding domain comprises a CDRL1 of SEQ ID NO: 174, a CDRL2 of AAS, a CDRL3 of SEQ ID NO: 175, a CDRH1 of SEQ ID NO: 176, a CDRH2 of SEQ ID NO: 177, and a CDRH3 of SEQ ID NO: 178.

In yet another embodiment, the PD-1 binding domain comprises a CDRL1 of SEQ ID NO: 183, a CDRL2 of WAS, a CDRL3 of SEQ ID NO: 184, a CDRH1 of SEQ ID NO: 185, a CDRH2 of SEQ ID NO: 186, and a CDRH3 of SEQ ID NO: 187.

In still another embodiment, the PD-1 binding domain comprises a CDRL1 of SEQ ID NO: 192, a CDRL2 of AAS, a CDRL3 of SEQ ID NO: 193, a CDRH1 of SEQ ID NO: 194, a CDRH2 of SEQ ID NO: 195, and a CDRH3 of SEQ ID NO: 196.

In certain embodiments, the CDRs provided herein are defined according to the IMGT or Kabat numbering system. In certain embodiments, the CDRs provided herein are defined according to the IMGT numbering system. In certain embodiments, the CDRs provided herein are defined according to the Kabat numbering system.

In another embodiment, the PD-1 binding domain comprises:
(i) a light chain variable region of SEQ ID NO: 116 and a heavy chain variable region of SEQ ID NO: 117;
(ii) a light chain variable region of SEQ ID NO: 125 and a heavy chain variable region of SEQ ID NO: 126;
(iii) a light chain variable region of SEQ ID NO: 134 and a heavy chain variable region of SEQ ID NO: 135;
(iv) a light chain variable region of SEQ ID NO: 143 and a heavy chain variable region of SEQ ID NO: 144;
(v) a light chain variable region of SEQ ID NO: 152 and a heavy chain variable region of SEQ ID NO: 153;
(vi) a light chain variable region of SEQ ID NO: 161 and a heavy chain variable region of SEQ ID NO: 162;
(vii) a light chain variable region of SEQ ID NO: 170 and a heavy chain variable region of SEQ ID NO: 171;
(viii) a light chain variable region of SEQ ID NO: 179 and a heavy chain variable region of SEQ ID NO: 180;
(ix) a light chain variable region of SEQ ID NO: 188 and a heavy chain variable region of SEQ ID NO: 189;
(x) a light chain variable region of SEQ ID NO: 197 and a heavy chain variable region of SEQ ID NO: 198.

In one embodiment, the PD-1 binding domain comprises a light chain variable region of SEQ ID NO: 116 and a heavy chain variable region of SEQ ID NO: 117. In another embodiment, the PD-1 binding domain comprises a light chain variable region of SEQ ID NO: 125 and a heavy chain variable region of SEQ ID NO: 126. In yet another embodiment, the PD-1 binding domain comprises a light chain variable region of SEQ ID NO: 134 and a heavy chain variable region of SEQ ID NO: 135. In yet another embodiment, the PD-1 binding domain comprises a light chain variable region of SEQ ID NO: 143 and a heavy chain variable region of SEQ ID NO: 144. In yet another embodiment, the PD-1 binding domain comprises a light chain variable region of SEQ ID NO: 152 and a heavy chain variable region of SEQ ID NO: 153. In yet another embodiment, the PD-1 binding domain comprises a light chain variable region of SEQ ID NO: 161 and a heavy chain variable region of SEQ ID NO: 162. In yet another embodiment, the PD-1 binding domain comprises a light chain variable region of SEQ ID NO: 170 and a heavy chain variable region of SEQ ID NO: 171. In yet another embodiment, the PD-1 binding domain comprises a light chain variable region of SEQ ID NO: 179 and a heavy chain variable region of SEQ ID NO: 180. In yet another embodiment, the PD-1 binding domain comprises a light chain variable region of SEQ ID NO: 188 and a heavy chain variable region of SEQ ID NO: 189. In still another embodiment, the PD-1 binding domain comprises a light chain variable region of SEQ ID NO: 197 and a heavy chain variable region of SEQ ID NO: 198.

In one embodiment, the PD-1 binding domain is a single-chain variable fragment (scFv), Fab, Fab′, F(ab)₂, F(ab′)₂, Fv, diabody, triabody, tetrabody, or minibody.

In another embodiment, the PD-1 binding domain is an scFv. In yet another embodiment, the PD-1 binding domain is an scFv comprising a light chain (V_{L}), a heavy chain (V_{H}), and optionally a peptide linker, wherein the C-terminal of the light chain is connected to the N-terminal of the light chain directly or via the peptide linker, or wherein the N-terminal of the light chain is connected to the C-terminal of the light chain directly or via the peptide linker. In yet another embodiment, the PD-1 binding domain is an scFv comprising a light chain (V_{L}), a heavy chain (V_{H}), and a peptide linker, wherein the C-terminal of the light chain is connected to the N-terminal of the light chain via the peptide linker, or wherein the N-terminal of the light chain is connected to the C-terminal of the light chain via the peptide linker. In yet another embodiment, the PD-1 binding domain is an scFv comprising a light chain (V_{L}), a heavy chain (V_{H}), and a peptide linker, wherein the C-terminal of the light chain is connected to the N-terminal of the light chain via the peptide linker. In yet another embodiment, the PD-1 binding domain is an scFv comprising a light chain (V_{L}), a heavy chain (V_{H}), and a peptide linker, wherein the N-terminal of the light chain is connected to the C-terminal of the light chain via the peptide linker. In certain embodiments, the peptide linker has the amino acid sequence of SEQ ID NO: 215 or 216.

In yet another embodiment, the PD-1 binding domain is a Fab. In yet another embodiment, the PD-1 binding domain is a Fab′. In yet another embodiment, the PD-1 binding domain is a F(ab)₂. In yet another embodiment, the PD-1 binding domain is a F(ab′)2. In yet another embodiment, the PD-1 binding domain is a Fv. In yet another embodiment, the PD-1 binding domain is a diabody. In yet another embodiment, the PD-1 binding domain is a triabody. In yet another embodiment, the PD-1 binding domain is a tetrabody. In yet another embodiment, the PD-1 binding domain is a minibody. In still another embodiment, the PD-1 binding domain is a V_{H}H single domain antibody.

In one embodiment, the PD-1 binding domain and the half-life extension domain are parts of an intact antibody comprising two light chains and two heavy chains.

In one embodiment, the intact antibody is an IgA, IgD, IgE, IgG, or IgM antibody. In another embodiment, the intact antibody is an IgA antibody. In yet another embodiment, the intact antibody is an IgD antibody. In yet another embodiment, the intact antibody is an IgE antibody. In yet another embodiment, the intact antibody is an IgG antibody. In still another embodiment, the intact antibody is an IgM antibody.

In one embodiment, the intact antibody is an IgA1, IgA2, IgG1, IgG2, IgG3, or IgG4 antibody. In another embodiment, the intact antibody is an IgA1 or IgA2. In yet another embodiment, the intact antibody is an IgA1. In yet another embodiment, the intact antibody is an IgA2. In yet another embodiment, the intact antibody is an IgG1, IgG2, IgG3, or IgG4 antibody. In yet another embodiment, the intact antibody is an IgG1 antibody. In yet another embodiment, the intact antibody is an IgG2 antibody. In yet another embodiment, the intact antibody is an IgG3 antibody. In still another embodiment, the intact antibody is an IgG4 antibody.

In one embodiment, the light chain is a kappa or lambda chain. In another embodiment, the light chain is a kappa chain. In yet another embodiment, the light chain is a lambda chain.

In one embodiment, the antibody comprises:
(i) a light chain of SEQ ID NO: 118 and a heavy chain of SEQ ID NO: 119;
(ii) a light chain of SEQ ID NO: 127 and a heavy chain of SEQ ID NO: 128;
(iii) a light chain of SEQ ID NO: 136 and a heavy chain of SEQ ID NO: 137;
(iv) a light chain of SEQ ID NO: 145 and a heavy chain of SEQ ID NO: 146;
(v) a light chain of SEQ ID NO: 154 and a heavy chain of SEQ ID NO: 155;
(vi) a light chain of SEQ ID NO: 163 and a heavy chain of SEQ ID NO: 164;
(vii) a light chain of SEQ ID NO: 172 and a heavy chain of SEQ ID NO: 173;
(viii) a light chain of SEQ ID NO: 181 and a heavy chain of SEQ ID NO: 182;
(ix) a light chain of SEQ ID NO: 190 and a heavy chain of SEQ ID NO: 191;
(x) a light chain of SEQ ID NO: 199 and a heavy chain of SEQ ID NO: 200.

In one embodiment, the antibody comprises a light chain of SEQ ID NO: 118 and a heavy chain of SEQ ID NO: 119. In another embodiment, the antibody comprises a light chain of SEQ ID NO: 127 and a heavy chain of SEQ ID NO: 128. In yet another embodiment, the antibody comprises a light chain of SEQ ID NO: 136 and a heavy chain of SEQ ID NO: 137. In yet another embodiment, the antibody comprises a light chain of SEQ ID NO: 145 and a heavy chain of SEQ ID NO: 146. In yet another embodiment, the antibody comprises a light chain of SEQ ID NO: 154 and a heavy chain of SEQ ID NO: 156. In yet another embodiment, the antibody comprises a light chain of SEQ ID NO: 163 and a heavy chain of SEQ ID NO: 164. In yet another embodiment, the antibody comprises a light chain of SEQ ID NO: 172 and a heavy chain of SEQ ID NO: 173. In yet another embodiment, the antibody comprises a light chain of SEQ ID NO: 181 and a heavy chain of SEQ ID NO: 182. In yet another embodiment, the antibody comprises a light chain of SEQ ID NO: 190 and a heavy chain of SEQ ID NO: 191. In still another embodiment, the antibody comprises a light chain of SEQ ID NO: 199 and a heavy chain of SEQ ID NO: 200.

### Peptide Linkers

In one embodiment, each peptide linker in a fusion protein provided herein independently comprises an amino acid sequence of GSG or one of SEQ ID NOs: 206 to 245. In another embodiment, each peptide linker in a fusion protein provided herein independently comprises an amino acid sequence of GSG or SEQ ID NO: 206, 207, or 208. In yet another embodiment, each peptide linker in a fusion protein provided herein independently comprises an amino acid sequence of SEQ ID NO: 209, 210, 211, or 212. In yet another embodiment, each peptide linker in a fusion protein provided herein independently comprises an amino acid sequence of SEQ ID NO: 213, 214, 215, or 216. In yet another embodiment, each peptide linker in a fusion protein provided herein independently comprises an amino acid sequence of SEQ ID NO: 217, 218, 219, or 220. In yet another embodiment, each peptide linker in a fusion protein provided herein independently comprises an amino acid sequence of SEQ ID NO: 221, 222, 223, or 224. In yet another embodiment, each peptide linker in a fusion protein provided herein independently comprises an amino acid sequence of SEQ ID NO: 225, 226, 227, or 228. In yet another embodiment, each peptide linker in a fusion protein provided herein independently comprises an amino acid sequence of SEQ ID NO: 229, 230, 231, or 232. In yet another embodiment, each peptide linker in a fusion protein provided herein independently comprises an amino acid sequence of SEQ ID NO: 233, 234, 235, or 236. In yet another embodiment, each peptide linker in a fusion protein provided herein independently comprises an amino acid sequence of SEQ ID NO: 237. In yet another embodiment, each peptide linker in a fusion protein provided herein is independently comprises an amino acid sequence of SEQ ID NO: 238 or 239. In yet another embodiment, each peptide linker in a fusion protein provided herein is independently comprises an amino acid sequence of SEQ ID NO: 240 or 241. In yet another embodiment, each peptide linker in a fusion protein provided herein is independently comprises an amino acid sequence of SEQ ID NO: 242 or 243. In still another embodiment, each peptide linker in a fusion protein provided herein is independently comprises an amino acid sequence of SEQ ID NO: 244 or 245.

### Pharmaceutical Compositions

In one embodiment, provided herein is a pharmaceutical composition comprising a fusion protein provided herein and a pharmaceutically acceptable excipient.

In one embodiment, the pharmaceutical composition is formulated as single dosage form.

In one embodiment, the pharmaceutical composition provided herein is a solid formulation. In another embodiment, the pharmaceutical composition provided herein is a lyophilized solid formulation. In yet another embodiment, the pharmaceutical composition provided herein is a solution. In still another embodiment, the pharmaceutical composition provided herein is an aqueous solution.

In one embodiment, the pharmaceutical composition provided herein is formulated in a dosage form for parenteral administration. In another embodiment, the pharmaceutical composition provided herein is formulated in a dosage form for intravenous administration. In yet another embodiment, the pharmaceutical composition provided herein is formulated in a dosage form for intramuscular administration. In yet another embodiment, the pharmaceutical composition provided herein is formulated in a dosage form for subcutaneous administration. In still another embodiment, the pharmaceutical composition provided herein is formulated in a dosage form for intratumoral administration.

### Methods of Use

In one embodiment, provided herein is a method of treating, preventing, or ameliorating one or more symptoms of a disorder, disease, or condition mediated by a PD-1 in a subject, comprising administering to the subject in need thereof a therapeutically effective amount of a fusion protein provided herein.

In certain embodiments, the disorder, disease, or condition mediated by a PD-1 is a proliferative disease.

In another embodiment, provided herein is a method of treating, preventing, or ameliorating one or more symptoms of a disorder, disease, or condition mediated by an IL-2 in a subject, comprising administering to the subject in need thereof a therapeutically effective amount of a fusion protein provided herein.

In certain embodiments, the disorder, disease, or condition mediated by an IL-2 is a proliferative disease.

In yet another embodiment, provided herein is a method for treating, preventing, or ameliorating a proliferative disease in a subject, comprising administering to the subject in need thereof a therapeutically effective amount of a fusion protein provided herein.

In one embodiment, the proliferative disease is cancer. In another embodiment, the proliferative disease is colorectal cancer.

In certain embodiments, the cancer is refractory and/or relapsed. In certain embodiments, the cancer is refractory. In certain embodiments, the cancer is relapsed. In certain embodiments, the cancer is metastatic. In certain embodiments, the cancer is resectable. In certain embodiments, the cancer is unresectable. In certain embodiments, the cancer is metastatic.

In certain embodiments, the cancer is drug-resistant. In certain embodiment, the cancer is multidrug-resistant. In certain embodiments, the cancer is resistant to a chemotherapy. In certain embodiments, the cancer is resistant to an immunotherapy. In certain embodiments, the cancer is resistant to a standard therapy for the cancer.

In certain embodiments, the subject is a mammal. In certain embodiments, the subject is a human.

In another embodiment, provided herein is a method of inhibiting the growth of a cell, comprising contacting the cell with an effective amount of a fusion protein provided herein. In certain embodiments, the cell is a cancerous cell. In certain embodiments, the cell is a human cancerous cell. In certain embodiments, the cell is a metastatic cancerous cell.

In yet another embodiment, provided herein is a method of activating an immune effector cell, comprising contacting the effector cell with an effective amount of a fusion protein provided herein. In certain embodiments, the effector cell is a human effector cell.

In certain embodiments, the therapeutically effective amount is ranging from about 0.001 mg per kg subject body weight every month (mg/kg per month) to 100 mg per kg subject body weight per day (mg/kg per day), from about 0.01 mg/kg per month to about 75 mg/kg per day, from about 0.1 mg/kg per month to about 50 mg/kg per day, from about 0.5 mg/kg per month to about 25 mg/kg per day, or from about 1 mg/kg per month to about 20 mg/kg per day, which can be administered in single or multiple doses. Within this range, the dosage can be ranging from about 0.005 mg/kg per month to about 0.05 mg/kg per day, from about 0.05 mg/kg per month to about 0.5 mg/kg per day, from about 0.5 mg/kg per month to about 5.0 mg/kg per day, from about 1 mg/kg per month to about 15 mg/kg per day, from about 1 mg/kg per month to about 20 mg/kg per day, or from about 1 mg/kg per month to about 50 mg/kg per day.

The disclosure will be further understood by the following non-limiting examples.

### EXAMPLES

### Example 1

### Cloning, Expression, and Purification of Fusion Proteins

The amino acid sequence of human IL-2 (hIL-2) was obtained from UNIPROT (hIL-2: P60568, 21-153 aa). A mutant human IL-2 was generated by introducing a mutation to abolish the CD25 binding as well as reducing the CD122 binding. The amino acid sequence of a human anti-PD-1 antibody (VH & VL) was obtained from the Therapeutic Antibody Database (TABS). The deoxyoligonucleotide (DNA) sequences encoding the mutant human IL2 and human anti-PD1 antibody were codon optimized for CHO cell expression.

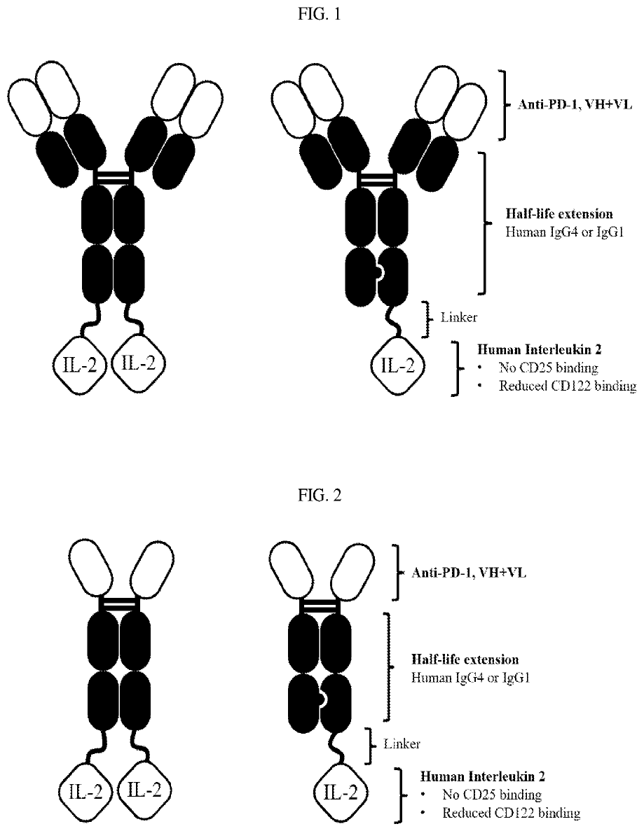

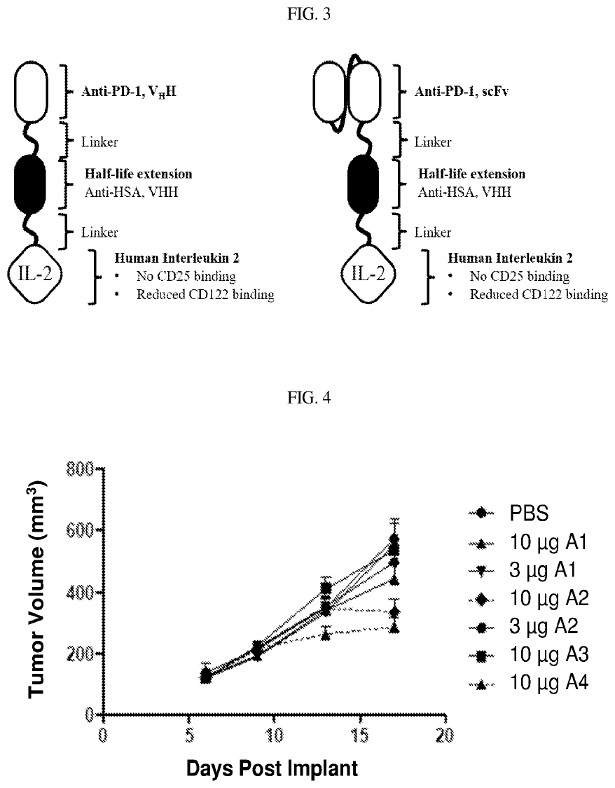

Certain configurations of fusion proteins containing (i) an hIL-2 mutein and (ii) a human anti-PD-1 antibody or a fragment thereof are illustrated in FIGS. 1 and 2. The deoxyoligonucleotide sequences encoding (i) the hIL-2 mutein, (ii) the human anti-PD-1 antibody or a fragment thereof, and (iii) other sequences such as a peptide linker were seamlessly assembled together by homology assembly cloning with commercially available kits. The oligonucleotides of each fusion protein were inserted into a UCOE® expression vector CET1019-AS-Puro for CHO cell expression.

The oligonucleotide sequence encoding a fusion protein was transiently expressed in EXPICHO™ cells. Briefly, on Day −1, EXPICHO-S™ cells were seeded at 3-4×10⁶ cells/mL with the EXPICHO™ expression medium in a vented Erlenmeyer shake flask. The flask was placed on a 125-rpm orbital shaker in a 37° C. incubator with 8% C02. On Day 0, plasmid DNA was mixed with the EXPIFECTAMINE™ CHO reagent. The mixture was then slowly added to the cells. After 16 hours, the cells were transferred to a 32° C. incubator with 5% C02. The cells were fed twice on Day 1 and Day 5 with the EXPICHO™ feed. The CHO cells were harvested on Day 8-12.

Each fusion protein produced in the CHO cells was purified by a two-step purification process: protein A affinity chromatography using protein A (e.g., AMSPHERE™ A3) resin and ion exchange chromatography (e.g., CAPTO™ Q ImpRes).

For the protein A affinity chromatography, a protein A affinity column was loaded with a clarified CHO medium and then washed once with 20 mM sodium phosphate at pH 7.5, once with 20 mM sodium phosphate with 0.5 M NaCl at pH 7.5, and once again with 20 mM sodium phosphate at pH 7.5. The fusion protein was eluted with 50 mM sodium acetate at pH 3.0 supplied with 1% isopropanol by volume.

The purified fusion protein was then buffer exchanged into 20 mM Tris-HCl at pH 8.5 in preparation of AKTA™ purification. The fusion protein was loaded onto 1 mL HiTrap CAPTO™ Q ImpRes column. The column was then washed with 20 mM Tris-HCl at pH 8.5 for 5 column volumes (CV) and eluted with 20 mM Tris-HCl at pH 8.5 plus 1 M NaCl by a gradient of 0-20% in 20 CV. The fusion protein was eluted off between 9˜12 mS/cm. Eluted fractions were pooled and buffer exchanged into a solution containing 20 mM sodium phosphate at pH 6.5 for storage.

### Example 2

### Stability Determination

The thermostability of purified fusion proteins was measured via dynamic light scattering using a DYNAPRO® NANOSTAR® dynamic light scattering detector. The fusion proteins were each buffer exchanged into 20 mM sodium phosphate at pH 6.5 and then incubated in a continuous temperature ramp ranging from 30 to 80° C. while measuring the radius of each fusion protein every 1° C. The results are summarized in Table 1. The fusion proteins were observed to have two Tₒₙₛₑₜₛ. Tₒₙₛₑₜ 1 was calculated to be between 55-59° C. depending on mutations in the IL-2 muteins. Tₒₙₛₑₜ 2 was calculated to be 70° C.

Anti-hPD-1/hIL-2 fusion protein A1 in a knobs-in-holes configuration has a light chain of SEQ ID NO: 136, a heavy chain of SEQ ID NO: 248, and an hIL-2 fused heavy chain of SEQ ID NO: 249, comprising an hIL-2 domain of SEQ ID NO: 42 and a peptide linker of SEQ ID NO: 215, wherein the N-terminus of the hIL-2 domain is connected to the C-terminus of the second heavy chain via the peptide linker. Anti-hPD-1/hIL-2 fusion protein A2 in a knobs-in-holes configuration has a light chain of SEQ ID NO: 136, a heavy chain of SEQ ID NO: 248, and an hIL-2 fused heavy chain of SEQ ID NO: 250, comprising an hIL-2 domain of SEQ ID NO: 54 and a peptide linker of SEQ ID NO: 215, wherein the N-terminus of the hIL-2 domain is connected to the C-terminus of the second heavy chain via the peptide linker. Anti-hPD-1/hIL-2 fusion protein A3 in a knobs-in-holes configuration has a light chain of SEQ ID NO: 136, a heavy chain of SEQ ID NO: 248, and an hIL-2 fused heavy chain of SEQ ID NO: 251, comprising an hIL-2 domain of SEQ ID NO: 68 and a peptide linker of SEQ ID NO: 215; and wherein the N-terminus of the hIL-2 domain is connected to the C-terminus of the second heavy chain via the peptide linker. Anti-hPD-1/hIL-2 fusion protein A4 in a knobs-in-holes configuration has a light chain of SEQ ID NO: 136, a heavy chain of SEQ ID NO: 248, and an hIL-2 fused heavy chain of SEQ ID NO: 252, comprising an hIL-2 domain of SEQ ID NO: 70 and a peptide linker of SEQ ID NO: 215, wherein the N-terminus of the hIL-2 domain is connected to the C-terminus of the second heavy chain via the peptide linker. Anti-hPD-1/hIL-2 fusion protein A5 in a knobs-in-holes configuration has a light chain of SEQ ID NO: 136, a heavy chain of SEQ ID NO: 248, and an hIL-2 fused heavy chain of SEQ ID NO: 253, comprising an hIL-2 domain of SEQ ID NO: 80 and a peptide linker of SEQ ID NO: 215, wherein the N-terminus of the hIL-2 domain is connected to the C-terminus of the second heavy chain via the peptide linker. Anti-hPD-1/hIL-2 fusion protein A6 in a knobs-in-holes configuration has a light chain of SEQ ID NO: 136, a heavy chain of SEQ ID NO: 248, and an hIL-2 fused heavy chain of SEQ ID NO: 254, comprising an hIL-2 domain of SEQ ID NO: 50 and a peptide linker of SEQ ID NO: 215, wherein the N-terminus of the hIL-2 domain is connected to the C-terminus of the second heavy chain via the peptide linker. Anti-hPD-1/hIL-2 fusion protein A19 in a knobs-in-holes configuration has a light chain of SEQ ID NO: 118, a heavy chain of SEQ ID NO: 201, and an hIL-2 fused heavy chain of SEQ ID NO: 203, comprising an hIL-2 domain of SEQ ID NO: 70 and a peptide linker of SEQ ID NO: 215, wherein the N-terminus of the hIL-2 domain is connected to the C-terminus of the second heavy chain via the peptide linker. Anti-hPD-1/hIL-2 fusion protein A20 in a knobs-in-holes configuration has a light chain of SEQ ID NO: 118, a heavy chain of SEQ ID NO: 201, and an hIL-2 fused heavy chain of SEQ ID NO: 204, comprising an hIL-2 domain of SEQ ID NO: 256 and a peptide linker of SEQ ID NO: 215, wherein the N-terminus of the hIL-2 domain is connected to the C-terminus of the second heavy chain via the peptide linker. Anti-hPD-1/hIL-2 fusion protein A21 has a light chain of SEQ ID NO: 118, and an hIL-2 fused heavy chain of SEQ ID NO: 205, comprising an hIL-2 domain of SEQ ID NO: 256 and a peptide linker of SEQ ID NO: 215, wherein the N-terminus of the hIL-2 domain is connected to the C-terminus of the second heavy chain via the peptide linker.

TABLE 1
Thermostability Determination of Fusion Proteins

| Protein | Tₒₙₛₑₜ 1 (° C.) | Tₒₙₛₑₜ 2 (° C.) |
|---|---|---|
| Anti-hPD-1/hIL-2 fusion protein A1 | 57.7 | 69.5 |
| Anti-hPD-1/hIL-2 fusion protein A2 | 58.2 | 70 |
| Anti-hPD-1/hIL-2 fusion protein A3 | 57.5 | 70.3 |
| Anti-hPD-1/hIL-2 fusion protein A4 | 56.8 | 70.6 |
| Anti-hPD-1/hIL-2 fusion protein A5 | 55.3 | 68.8 |
| Anti-hPD-1/hIL-2 fusion protein A6 | 57.9 | 69.8 |
| Anti-hPD-1/hIL-2 fusion protein A19 | 62.5 | N.A. |
| Anti-hPD-1/hIL-2 fusion protein A20 | 62.3 | N.A. |
| Anti-hPD-1/hIL-2 fusion protein A21 | 62 | N.A. |

### Example 3

### Binding Studies of IL-2 Muteins to CD25 and CD122

OCTET® RED96 was used to characterize the interactions of wildtype IL-2 and IL-2 muteins with CD25 and CD122. Briefly, an CD25- or CD122-Fc fusion protein was loaded onto an anti-human IgG Fc capture (AHC) biosensor. The biosensor was then dipped into a solution containing wild-type human IL-2 or an IL-2 mutein at 400 nM. The results are summarized in Table 2. All IL-2 muteins tested were found to have abolished or significantly reduced CD25 binding.

TABLE 2
Interactions of Wildtype IL-2 and
IL-2 Muteins with CD25 and CD122

| | SEQ | Binding Response | |
|---|---|---|---|
| Fusion Protein | ID NO: | CD25 | CD122 |
| Wildtype hIL-2 | 2 | 1 | 1 |
| hIL-2 Mutein (R38N, L40T, C125S) | 6 | <0.05 | >0.5 |
| hIL-2 Mutein (K32E, R38E, F42K, C125S) | 16 | <0.05 | >0.5 |
| hIL-2 Mutein (K32E, F42C, V69C, C125S) | 28 | <0.05 | >0.5 |
| IL-2-IL-15 Chimera 1 (C125S) | 42 | <0.05 | >0.5 |
| IL-2-IL-15 Chimera 5 (E15K, C125S) | 46 | <0.05 | >0.5 |
| IL-2-IL-15 Chimera 11 (H16E, C125S) | 52 | <0.05 | <0.2 |
| IL-2-IL-15 Chimera 15 (H16I, C125S) | 56 | <0.05 | <0.1 |
| IL-2-IL-15 Chimera 17 (H16V, C125S) | 58 | <0.05 | <0.1 |
| IL-2-IL-15 Chimera 19 (D20T, C125S) | 60 | <0.05 | <0.1 |
| IL-2-IL-15 Chimera 21 (D20A, C125S) | 62 | <0.05 | <0.1 |
| IL-2-IL-15 Chimera 23 (D20E, C125S) | 64 | <0.05 | <0.1 |
| IL-2-IL-15 Chimera 25 (D20K, C125S) | 66 | <0.05 | <0.1 |
| IL-2-IL-15 Chimera 27 (N88A, C125S) | 68 | <0.05 | <0.1 |
| IL-2-IL-15 Chimera 29 (I92A, C125S) | 70 | <0.05 | <0.1 |
| IL-2-IL-15 Chimera 29 (I92G, C125S) | 72 | <0.05 | <0.2 |
| IL-2-IL-15 Chimera 29 (I92D, C125S) | 74 | <0.05 | <0.1 |
| IL-2-IL-15 Chimera 29 (I92E, C125S) | 76 | <0.05 | <0.1 |

### Example 4

### Antitumor Activity of Anti-PD-1/IL-2 Fusion Protein in a Xenograft Mouse Model

MC38 cells are cultured and maintained in DMEM media supplemented with 10% fetal bovine serum, GLUTAMAX™, non-essential amino acids (NEAA), sodium pyruvate, and penicillin/streptomycin. The cells are trypsinized, washed with the media, and counted. The cells are diluted with PBS and 5×10⁵ cells in PBS (50 μL) are injected subcutaneously into anesthetized C57BL/6 mice using an 18-gauge needle. A stock solution of a fusion protein is diluted in PBS on the day of dosing and the mice are dosed intraperitoneally with PBS (control), a mouse anti-PD-1 (anti-mPD-1) (1 to 20 μg), a corresponding hIL-2 fusion protein (1 to 100 μg), a combination of the anti-mPD-1 (1 to 100 μg) and corresponding hIL-2 fusion protein (1 to 100 μg), or an anti-mPD-1/hIL-2 fusion protein (20 μg) in PBS (100 μL) twice a week for two weeks. Tumor sizes (length (L) and width (W)) are measured twice per week using a digital caliper, and the tumor volume is calculated (L×W×W)/2. The hIL-2 fusion protein comprises an hIL-2 domain described herein and an anti-HSA domain, wherein the C-terminus of the hIL-2 domain is connected to the N-terminus of the anti-HSA domain via the peptide linker. The anti-mPD-1/hIL-2 fusion protein is in a knobs-in-holes configuration, comprising the same hIL-2 domain and a peptide linker, wherein the N-terminus of the hIL-2 domain is connected to the C-terminus of the second heavy chain via the peptide linker.

### Example 5. Antitumor Activity of Anti-PD-1/IL-2 Fusion Protein in a Xenograft Model Using PD-1 Knock-In Mice

MC38 cells or B16F10 cells are cultured and maintained in DMEM media supplemented with 10% fetal bovine serum, GLUTAMAX™, non-essential amino acids (NEAA), sodium pyruvate, and penicillin/streptomycin. The cells are trypsinized, washed with the media, and counted. The cells are diluted with PBS and 5×10⁵ cells in PBS (50 μL) are injected subcutaneously into anesthetized C57BL/6 mice with human PD-1 knock-in using an 18-gauge needle. A stock solution of a fusion protein is diluted in PBS on the day of dosing and the mice are dosed intraperitoneally with PBS (control), a human anti-PD-1 (anti-hPD-1) (1 to 20 g), a corresponding hIL-2 fusion protein (1 to 100 μg), a combination of the anti-hPD-1 (1 to 100 μg) and corresponding hIL-2 fusion protein (1 to 100 μg), or an anti-hPD-1/hIL-2 fusion protein (20 ag) in PBS (100 μL) twice a week for two weeks. Tumor sizes (length (L) and width (W)) are measured twice per week using a digital caliper, and the tumor volume is calculated (L×W×W)/2. The hIL-2 fusion protein comprises an hIL-2 domain described herein and an anti-HSA domain, wherein the C-terminus of the hIL-2 domain is connected to the N-terminus of the anti-HSA domain via the peptide linker. The anti-hPD-1/hIL-2 fusion protein is in a knobs-in-holes configuration, comprising the same hIL-2 domain and a peptide linker, wherein the N-terminus of the hIL-2 domain is connected to the C-terminus of the second heavy chain via the peptide linker.

### Example 6

### Antitumor Activity of Anti-PD-1/IL-2 Fusion Protein in a Xenograft Mouse Model

CT26 mouse cells are cultured and maintained in RPMI media supplemented with 10% fetal bovine serum, GLUTAMAX™, and penicillin/streptomycin. The cells are trypsinized, washed with media, and counted. The cells are diluted with PBS and 1×10⁶ cells in PBS (100 μL) are injected subcutaneously into anesthetized BALB/c mice using an 18-gauge needle. A stock solution of a fusion protein is diluted in PBS on the day of dosing and the mice are dosed intraperitoneally twice per week for two weeks with (i) PBS (control), an anti-mPD-1 (20 μg), an anti-mPD-1/hIL-2 fusion protein (5 or 20 μg), or an anti-mPD-1/mIL-2 fusion protein (5 or 20 g). Tumor sizes (length (L) and width (W)) are measured twice per week using a digital caliper, and the tumor volume is calculated (L×W×W)/2.

### Example 7

### Antitumor Activity of Anti-PD-1/IL-2 Fusion Protein in a Xenograft Mouse Model

HT-29 cells were cultured and maintained in McCoys 5a media supplemented with 10% fetal bovine serum and penicillin/streptomycin. The cells were trypsinized, washed with media, counted, and washed with PBS. The cell suspension (1×10⁶ cells in PBS (100 μL)) was injected subcutaneously into anesthetized NCG mice using a 27-gauge needle. After 6 days, human PBMCs (1×10⁷ cells in PBS (100 μL)) were injected into the tail vein of each mouse. A stock solution of a fusion protein was diluted in PBS on the day of dosing and the mice were dosed intraperitoneally twice per week for two weeks. Tumor sizes (length (L) and width (W)) were measured twice per week using a digital caliper, and the tumor volume was calculated (L×W×W)/2. The results are shown in FIG. 4.

### Example 8

### Effect of Anti-PD-1/IL-2 Fusion Proteins on STAT5 Signaling

Anti-PD-1/IL-2 fusion proteins were evaluated for their effect on pSTAT5 signaling via its IL-2 domain in CD3 T-cells. Briefly, CD3 T-cells (100,000) were treated with an anti-PD-1/IL-2 fusion protein for 30 min at 37 C and 5% CO₂ in Hanks balanced salt solution containing 10 mM HEPES. Phospho-STAT5 was measured using a phosphor-STAT5 (Tyr694) HTRF assay. The signal ratio at 665 nm/620 nm was multiplied by 1,000 and the data was analyzed with global fitting to determine EC₅₀ values. The results are summarized in Table 3, where “A” represents an EC₅₀ value of no greater than 10 nM, “B” represents an EC₅₀ value of greater than 10 nM and no greater than 100 nM, “C” represents an EC₅₀ value of greater than 100 nM and no greater than 1,000 nM, and “D” represents an EC₅₀ value of greater than 1,000 nM.

TABLE 3
STAT5 Signaling in CD3⁺ T-Cells

| | EC₅₀ (nM) | | |
|---|---|---|---|
| | Mouse T-cell | Human CD3⁺ | Human CD3⁺ |
| Fusion Protein | (CTLL2) | T-cells | PD1⁺ T-cells |
| Anti-hPD-1/hIL-2 A1 | ND | A | A |
| Anti-hPD-1/hIL-2 A2 | ND | C | B |
| Anti-hPD-1/hIL-2 A3 | B | C | B |
| Anti-hPD-1/hIL-2 A4 | A | B | A |
| Anti-hPD-1/hIL-2 A5 | ND | B | ND |
| Anti-hPD-1/hIL-2 A6 | ND | A | A |
| Anti-hPD-1/hIL-2 A19 | B | B | A |
| Anti-hPD-1/hIL-2 A20 | D | D | C |
| Anti-hPD-1/hIL-2 A21 | ND | C | N.D. |

Sequences described herein are provided in the sequence table below.

SEQUENCE TABLE

| SEQ ID NO: | Description | Amino Acid Sequence |
|---|---|---|
| 1 | hIL-2 | APTSSSTKKTQLQLEHLLLDLQMILNGINNYKNPKL |
| | | TRMLTFKFYMPKKATELKHLQCLEEELKPLEEVLN |
| | | LAQSKNFHLRPRDLISNINVIVLELKGSETTFMCEY |
| | | ADETATIVEFLNRWITFCQSIISTLT |
| 2 | hIL-2 (C125S) | APTSSSTKKTQLQLEHLLLDLQMILNGINNYKNPKL |
| | | TRMLTFKFYMPKKATELKHLQCLEEELKPLEEVLN |
| | | LAQSKNFHLRPRDLISNINVIVLELKGSETTFMCEY |
| | | ADETATIVEFLNRWITFSQSIISTLT |
| 3 | hIL-2 (C125A) | APTSSSTKKTQLQLEHLLLDLQMILNGINNYKNPKL |
| | | TRMLTFKFYMPKKATELKHLQCLEEELKPLEEVLN |
| | | LAQSKNFHLRPRDLISNINVIVLELKGSETTFMCEY |
| | | ADETATIVEFLNRWITFAQSIISTLT |
| 4 | hIL-2 (T3A, C125S) | APASSSTKKTQLQLEHLLLDLQMILNGINNYKNPK |
| | | LTRMLTFKFYMPKKATELKHLQCLEEELKPLEEVL |
| | | NLAQSKNFHLRPRDLISNINVIVLELKGSETTFMCE |
| | | YADETATIVEFLNRWITFSQSIISTLT |
| 5 | hIL-2 (T3A, C125A) | APASSSTKKTQLQLEHLLLDLQMILNGINNYKNPK |
| | | LTRMLTFKFYMPKKATELKHLQCLEEELKPLEEVL |
| | | NLAQSKNFHLRPRDLISNINVIVLELKGSETTFMCE |
| | | YADETATIVEFLNRWITFAQSIISTLT |
| 6 | hIL-2 Mutein | APTSSSTKKTQLQLEHLLLDLQMILNGINNYKNPKL |
| | (R38N, L40T, C125S) | TNMTTFKFYMPKKATELKHLQCLEEELKPLEEVLN |
| | | LAQSKNFHLRPRDLISNINVIVLELKGSETTFMCEY |
| | | ADETATIVEFLNRWITFSQSIISTLT |
| 7 | hIL-2 Mutein | APASSSTKKTQLQLEHLLLDLQMILNGINNYKNPK |
| | (T3A, R38N, L40T, C125S) | LTNMTTFKFYMPKKATELKHLQCLEEELKPLEEVL |
| | | NLAQSKNFHLRPRDLISNINVIVLELKGSETTFMCE |
| | | YADETATIVEFLNRWITFSQSIISTLT |
| 8 | hIL-2 Mutein | APTSSSTKKTQLQLKHLLLDLQMILNGINNYKNPK |
| | (E15K, R38N, L40T, C125S) | LTNMTTFKFYMPKKATELKHLQCLEEELKPLEEVL |
| | | NLAQSKNFHLRPRDLISNINVIVLELKGSETTFMCE |
| | | YADETATIVEFLNRWITFSQSIISTLT |
| 9 | hIL-2 Mutein | APASSSTKKTQLQLKHLLLDLQMILNGINNYKNPK |
| | (T3A, E15K, R38N, L40T, | LTNMTTFKFYMPKKATELKHLQCLEEELKPLEEVL |
| | C125S) | NLAQSKNFHLRPRDLISNINVIVLELKGSETTFMCE |
| | | YADETATIVEFLNRWITFSQSIISTLT |
| 10 | hIL-2 Mutein | APTSSSTKKTQLQLKHLLLDLQMILNGINNYENPKL |
| | (E15K, K32E, R38N, L40T, | TNMTTFKFYMPKKATELKHLQCLEEELKPLEEVLN |
| | C125S) | LAQSKNFHLRPRDLISNINVIVLELKGSETTFMCEY |
| | | ADETATIVEFLNRWITFSQSIISTLT |
| 11 | hIL-2 Mutein | APASSSTKKTQLQLKHLLLDLQMILNGINNYENPK |
| | (T3A, E15K, K32E, R38N, | LTNMTTFKFYMPKKATELKHLQCLEEELKPLEEVL |
| | L40T, C125S) | NLAQSKNFHLRPRDLISNINVIVLELKGSETTFMCE |
| | | YADETATIVEFLNRWITFSQSIISTLT |
| 12 | hIL-2 Mutein | APTSSSTKKTQLQLKHLLLDLQMILNGINNYKNPK |
| | (E15K, R38N, L40T, K76E, | LTNMTTFKFYMPKKATELKHLQCLEEELKPLEEVL |
| | C125S) | NLAQSENFHLRPRDLISNINVIVLELKGSETTFMCE |
| | | YADETATIVEFLNRWITFSQSIISTLT |
| 13 | hIL-2 Mutein | APASSSTKKTQLQLKHLLLDLQMILNGINNYKNPK |
| | (T3A, E15K, R38N, L40T, | LTNMTTFKFYMPKKATELKHLQCLEEELKPLEEVL |
| | K76E, C125S) | NLAQSENFHLRPRDLISNINVIVLELKGSETTFMCE |
| | | YADETATIVEFLNRWITFSQSIISTLT |
| 14 | hIL-2 Mutein | APTSSSTKKTQLQLEHLLLDLQMILNGINNYENPKL |
| | (K32E, R38E, F42A, C125S) | TEMLTAKFYMPKKATELKHLQCLEEELKPLEEVLN |
| | | LAQSKNFHLRPRDLISNINVIVLELKGSETTFMCEY |
| | | ADETATIVEFLNRWITFSQSIISTLT |
| 15 | hIL-2 Mutein | APASSSTKKTQLQLEHLLLDLQMILNGINNYENPKL |
| | (T3A, K32E, R38E, F42A, | TEMLTAKFYMPKKATELKHLQCLEEELKPLEEVLN |
| | C125S) | LAQSKNFHLRPRDLISNINVIVLELKGSETTFMCEY |
| | | ADETATIVEFLNRWITFSQSIISTLT |
| 16 | hIL-2 Mutein | APTSSSTKKTQLQLEHLLLDLQMILNGINNYENPKL |
| | (K32E, R38E, F42K, C125S) | TEMLTKKFYMPKKATELKHLQCLEEELKPLEEVLN |
| | | LAQSKNFHLRPRDLISNINVIVLELKGSETTFMCEY |
| | | ADETATIVEFLNRWITFSQSIISTLT |
| 17 | hIL-2 Mutein | APASSSTKKTQLQLEHLLLDLQMILNGINNYENPKL |
| | (T3A, K32E, R38E, F42K, | TEMLTKKFYMPKKATELKHLQCLEEELKPLEEVLN |
| | C125S) | LAQSKNFHLRPRDLISNINVIVLELKGSETTFMCEY |
| | | ADETATIVEFLNRWITFSQSIISTLT |
| 18 | hIL-2 Mutein | APTSSSTKKTQLQLKHLLLDLQMILNGINNYENPKL |
| | (E15K, K32E, R38E, F42K, | TEMLTKKFYMPKKATELKHLQCLEEELKPLEEVLN |
| | C125S) | LAQSKNFHLRPRDLISNINVIVLELKGSETTFMCEY |
| | | ADETATIVEFLNRWITFSQSIISTLT |
| 19 | hIL-2 Mutein | APASSSTKKTQLQLKHLLLDLQMILNGINNYENPK |
| | (T3A, E15K, K32E, R38E, | LTEMLTKKFYMPKKATELKHLQCLEEELKPLEEVL |
| | F42K, C125S) | NLAQSKNFHLRPRDLISNINVIVLELKGSETTFMCE |
| | | YADETATIVEFLNRWITFSQSIISTLT |
| 20 | hIL-2 Mutein | APTSSSTKKTQLQLEHLLLDLQMILNGINNYKNPKL |
| | (R38E, F42A, K76E, C125S) | TEMLTAKFYMPKKATELKHLQCLEEELKPLEEVLN |
| | | LAQSENFHLRPRDLISNINVIVLELKGSETTFMCEY |
| | | ADETATIVEFLNRWITFSQSIISTLT |
| 21 | hIL-2 Mutein | APASSSTKKTQLQLEHLLLDLQMILNGINNYKNPK |
| | (T3A, R38E, F42A, K76E, | LTEMLTAKFYMPKKATELKHLQCLEEELKPLEEVL |
| | C125S) | NLAQSENFHLRPRDLISNINVIVLELKGSETTFMCE |
| | | YADETATIVEFLNRWITFSQSIISTLT |
| 22 | hIL-2 Mutein | APTSSSTKKTQLQLEHLLLDLQMILNGINNYKNPKL |
| | (R38E, F42K, K76E, C125S) | TEMLTKKFYMPKKATELKHLQCLEEELKPLEEVLN |
| | | LAQSENFHLRPRDLISNINVIVLELKGSETTFMCEY |
| | | ADETATIVEFLNRWITFSQSIISTLT |
| 23 | hIL-2 Mutein | APASSSTKKTQLQLEHLLLDLQMILNGINNYKNPK |
| | (T3A, R38E, F42K, K76E, | LTEMLTKKFYMPKKATELKHLQCLEEELKPLEEVL |
| | C125S) | NLAQSENFHLRPRDLISNINVIVLELKGSETTFMCE |
| | | YADETATIVEFLNRWITFSQSIISTLT |
| 24 | hIL-2 Mutein | APTSSSTKKTQLQLKHLLLDLQMILNGINNYKNPK |
| | (E15K, R38E, F42K, K76E, | LTEMLTKKFYMPKKATELKHLQCLEEELKPLEEVL |
| | C125S) | NLAQSENFHLRPRDLISNINVIVLELKGSETTFMCE |
| | | YADETATIVEFLNRWITFSQSIISTLT |
| 25 | hIL-2 Mutein | APASSSTKKTQLQLKHLLLDLQMILNGINNYKNPK |
| | (T3A, E15K, R38E, F42K, | LTEMLTKKFYMPKKATELKHLQCLEEELKPLEEVL |
| | K76E, C125S) | NLAQSENFHLRPRDLISNINVIVLELKGSETTFMCE |
| | | YADETATIVEFLNRWITFSQSIISTLT |
| 26 | hIL-2 Mutein | APTSSSTKKTQLQLEHLLLDLQMILNGINNYKNPKL |
| | (F42C, V69C, C125S) | TRMLTCKFYMPKKATELKHLQCLEEELKPLEECLN |
| | | LAQSKNFHLRPRDLISNINVIVLELKGSETTFMCEY |
| | | ADETATIVEFLNRWITFSQSIISTLT |
| 27 | hIL-2 Mutein | APASSSTKKTQLQLEHLLLDLQMILNGINNYKNPK |
| | (T3A, F42C, V69C, C125S) | LTRMLTCKFYMPKKATELKHLQCLEEELKPLEECL |
| | | NLAQSKNFHLRPRDLISNINVIVLELKGSETTFMCE |
| | | YADETATIVEFLNRWITFSQSIISTLT |
| 28 | hIL-2 Mutein | APTSSSTKKTQLQLEHLLLDLQMILNGINNYENPKL |
| | (K32E, F42C, V69C, C125S) | TRMLTCKFYMPKKATELKHLQCLEEELKPLEECLN |
| | | LAQSKNFHLRPRDLISNINVIVLELKGSETTFMCEY |
| | | ADETATIVEFLNRWITFSQSIISTLT |
| 29 | hIL-2 Mutein | APASSSTKKTQLQLEHLLLDLQMILNGINNYENPKL |
| | (T3A, K32E, F42C, V69C, | TRMLTCKFYMPKKATELKHLQCLEEELKPLEECLN |
| | C125S) | LAQSKNFHLRPRDLISNINVIVLELKGSETTFMCEY |
| | | ADETATIVEFLNRWITFSQSIISTLT |
| 30 | hIL-2 Mutein | APTSSSTKKTQLQLEHLLLDLQMILNGINNYKNPKL |
| | (F42C, V69C, K76E, C125S) | TRMLTCKFYMPKKATELKHLQCLEEELKPLEECLN |
| | | LAQSENFHLRPRDLISNINVIVLELKGSETTFMCEY |
| | | ADETATIVEFLNRWITFSQSIISTLT |
| 31 | hIL-2 Mutein | APASSSTKKTQLQLEHLLLDLQMILNGINNYKNPK |
| | (T3A, F42C, V69C, K76E, | LTRMLTCKFYMPKKATELKHLQCLEEELKPLEECL |
| | C125S) | NLAQSENFHLRPRDLISNINVIVLELKGSETTFMCE |
| | | YADETATIVEFLNRWITFSQSIISTLT |
| 32 | hIL-2 Mutein | APTSSSTKKTQLQLKHLLLDLQMILNGINNYKNPK |
| | (E15K, F42C, V69C, C125S) | LTRMLTCKFYMPKKATELKHLQCLEEELKPLEECL |
| | | NLAQSKNFHLRPRDLISNINVIVLELKGSETTFMCE |
| | | YADETATIVEFLNRWITFSQSIISTLT |
| 33 | hIL-2 Mutein | APASSSTKKTQLQLKHLLLDLQMILNGINNYKNPK |
| | (T3A, E15K, F42C, V69C, | LTRMLTCKFYMPKKATELKHLQCLEEELKPLEECL |
| | C125S) | NLAQSKNFHLRPRDLISNINVIVLELKGSETTFMCE |
| | | YADETATIVEFLNRWITFSQSIISTLT |
| 34 | hIL-2 Mutein | APTSSSTKKTQLQLKHLLLDLQMILNGINNYENPKL |
| | (E15K, K32E, F42C, V69C, | TRMLTCKFYMPKKATELKHLQCLEEELKPLEECLN |
| | C125S) | LAQSKNFHLRPRDLISNINVIVLELKGSETTFMCEY |
| | | ADETATIVEFLNRWITFSQSIISTLT |
| 35 | hIL-2 Mutein | APASSSTKKTQLQLKHLLLDLQMILNGINNYENPK |
| | (T3A, E15K, K32E, F42C, | LTRMLTCKFYMPKKATELKHLQCLEEELKPLEECL |
| | V69C, C125S) | NLAQSKNFHLRPRDLISNINVIVLELKGSETTFMCE |
| | | YADETATIVEFLNRWITFSQSIISTLT |
| 36 | hIL-2 Mutein | APTSSSTKKTQLQLKHLLLDLQMILNGINNYKNPK |
| | (E15K, F42C, V69C, K76E, | LTRMLTCKFYMPKKATELKHLQCLEEELKPLEECL |
| | C125S) | NLAQSENFHLRPRDLISNINVIVLELKGSETTFMCE |
| | | YADETATIVEFLNRWITFSQSIISTLT |
| 37 | hIL-2 Mutein | APASSSTKKTQLQLKHLLLDLQMILNGINNYKNPK |
| | (T3A, E15K, F42C, V69C, | LTRMLTCKFYMPKKATELKHLQCLEEELKPLEECL |
| | K76E, C125S) | NLAQSENFHLRPRDLISNINVIVLELKGSETTFMCE |
| | | YADETATIVEFLNRWITFSQSIISTLT |
| 38 | hIL-2 Fragment | NNYKNPKLTRMLTFKFYMPKKA |
| | (N29 to A50) | |
| 39 | hIL-2 Fragment | NNYKNPKLTRML |
| | (N29 to L40) | |
| 40 | IL-15 Hinge Fragment- | QSMEIDAT |
| | containing Peptide 1 | |
| 41 | IL-15 Hinge Fragment- | QSMHIDAT |
| | containing peptide 2 | |
| 42 | IL-2-IL-15 Chimera 1 | APTSSSTKKTQLQLEHLLLDLQMILNGIQSMEIDAT |
| | (C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 43 | IL-2-IL-15 Chimera 2 | APASSSTKKTQLQLEHLLLDLQMILNGIQSMEIDAT |
| | (T3A, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 44 | IL-2-IL-15 Chimera 3 | APTSSSTKKTQLQLEHLLLDLQMILNGIQSMHIDAT |
| | (E32H, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 45 | IL-2-IL-15 Chimera 4 | APASSSTKKTQLQLEHLLLDLQMILNGIQSMHIDAT |
| | (T3A, E32H, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 46 | IL-2-IL-15 Chimera 5 | APTSSSTKKTQLQLKHLLLDLQMILNGIQSMEIDAT |
| | (E15K, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 47 | IL-2-IL-15 Chimera 6 | APASSSTKKTQLQLKHLLLDLQMILNGIQSMEIDAT |
| | (T3A, E15K, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 48 | IL-2-IL-15 Chimera 7 | APTSSSTKKTQLQLKHLLLDLQMILNGIQSMHIDAT |
| | (E15K, E32H, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 49 | IL-2-IL-15 Chimera 8 | APASSSTKKTQLQLKHLLLDLQMILNGIQSMHIDAT |
| | (T3A, E15K, E32H, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 50 | IL-2-IL-15 Chimera 9 | APTSSSTKKTQLQLKHLLLDLQMILNGIQSMHIDAT |
| | (E15K, E32H, S87D, C125S) | TFKFYMPKKATELKHLQCLEEELKPLEEVLNLAQS |
| | | KNFHLRPRDLIDNINVIVLELKGSETTFMCEYADET |
| | | ATIVEFLNRWITFSQSIISTLT |
| 51 | IL-2-IL-15 Chimera 10 | APASSSTKKTQLQLKHLLLDLQMILNGIQSMHIDAT |
| | (T3A, E15K, E32H, S87D, | TFKFYMPKKATELKHLQCLEEELKPLEEVLNLAQS |
| | C125S) | KNFHLRPRDLIDNINVIVLELKGSETTFMCEYADET |
| | | ATIVEFLNRWITFSQSIISTLT |
| 52 | IL-2-IL-15 Chimera 11 | APTSSSTKKTQLQLEELLLDLQMILNGIQSMEIDAT |
| | (H16E, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 53 | IL-2-IL-15 Chimera 12 | APASSSTKKTQLQLEELLLDLQMILNGIQSMEIDAT |
| | (T3A, H16E, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 54 | IL-2-IL-15 Chimera 13 | APTSSSTKKTQLQLEFLLLDLQMILNGIQSMEIDAT |
| | (H16F, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 55 | IL-2-IL-15 Chimera 14 | APASSSTKKTQLQLEFLLLDLQMILNGIQSMEIDAT |
| | (T3A, H16F, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 56 | IL-2-IL-15 Chimera 15 | APTSSSTKKTQLQLEILLLDLQMILNGIQSMEIDATT |
| | (H16I, C125S) | FKFYMPKKATELKHLQCLEEELKPLEEVLNLAQSK |
| | | NFHLRPRDLISNINVIVLELKGSETTFMCEYADETA |
| | | TIVEFLNRWITFSQSIISTLT |
| 57 | IL-2-IL-15 Chimera 16 | APASSSTKKTQLQLEILLLDLQMILNGIQSMEIDATT |
| | (T3A, H16I, C125S) | FKFYMPKKATELKHLQCLEEELKPLEEVLNLAQSK |
| | | NFHLRPRDLISNINVIVLELKGSETTFMCEYADETA |
| | | TIVEFLNRWITFSQSIISTLT |
| 58 | IL-2-IL-15 Chimera 17 | APTSSSTKKTQLQLEVLLLDLQMILNGIQSMEIDAT |
| | (H16V, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 59 | IL-2-IL-15 Chimera 18 | APASSSTKKTQLQLEVLLLDLQMILNGIQSMEIDAT |
| | (T3A, H16V, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 60 | IL-2-IL-15 Chimera 19 | APTSSSTKKTQLQLEHLLLTLQMILNGIQSMEIDAT |
| | (D20T, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 61 | IL-2-IL-15 Chimera 20 | APASSSTKKTQLQLEHLLLTLQMILNGIQSMEIDAT |
| | (T3A, D20T, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 62 | IL-2-IL-15 Chimera 21 | APTSSSTKKTQLQLEHLLLALQMILNGIQSMEIDAT |
| | (D20A, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 63 | IL-2-IL-15 Chimera 22 | APASSSTKKTQLQLEHLLLALQMILNGIQSMEIDAT |
| | (T3A, D20A, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 64 | IL-2-IL-15 Chimera 23 | APTSSSTKKTQLQLEHLLLELQMILNGIQSMEIDAT |
| | (D20E, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 65 | IL-2-IL-15 Chimera 24 | APASSSTKKTQLQLEHLLLELQMILNGIQSMEIDAT |
| | (T3A, D20E, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 66 | IL-2-IL-15 Chimera 25 | APTSSSTKKTQLQLEHLLLKLQMILNGIQSMEIDAT |
| | (D20K, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 67 | IL-2-IL-15 Chimera 26 | APASSSTKKTQLQLEHLLLKLQMILNGIQSMEIDAT |
| | (T3A, D20K, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 68 | IL-2-IL-15 Chimera 27 | APTSSSTKKTQLQLEHLLLDLQMILNGIQSMEIDAT |
| | (N88A, C125S) | TFKFYMPKKATELKHLQCLEEELKPLEEVLNLAQS |
| | | KNFHLRPRDLISAINVIVLELKGSETTFMCEYADET |
| | | ATIVEFLNRWITFSQSIISTLT |
| 69 | IL-2-IL-15 Chimera 28 | APASSSTKKTQLQLEHLLLDLQMILNGIQSMEIDAT |
| | (T3A, N88A, C125S) | TFKFYMPKKATELKHLQCLEEELKPLEEVLNLAQS |
| | | KNFHLRPRDLISAINVIVLELKGSETTFMCEYADET |
| | | ATIVEFLNRWITFSQSIISTLT |
| 70 | IL-2-IL-15 Chimera 29 | APTSSSTKKTQLQLEHLLLDLQMILNGIQSMEIDAT |
| | (192A, C125S) | TFKFYMPKKATELKHLQCLEEELKPLEEVLNLAQS |
| | | KNFHLRPRDLISNINVAVLELKGSETTFMCEYADET |
| | | ATIVEFLNRWITFSQSIISTLT |
| 71 | IL-2-IL-15 Chimera 30 | APASSSTKKTQLQLEHLLLDLQMILNGIQSMEIDAT |
| | (T3A, 192A, C125S) | TFKFYMPKKATELKHLQCLEEELKPLEEVLNLAQS |
| | | KNFHLRPRDLISNINVAVLELKGSETTFMCEYADET |
| | | ATIVEFLNRWITFSQSIISTLT |
| 72 | IL-2-IL-15 Chimera 31 | APTSSSTKKTQLQLEHLLLDLQMILNGIQSMEIDAT |
| | (192G, C125S) | TFKFYMPKKATELKHLQCLEEELKPLEEVLNLAQS |
| | | KNFHLRPRDLISNINVGVLELKGSETTFMCEYADET |
| | | ATIVEFLNRWITFSQSIISTLT |
| 73 | IL-2-IL-15 Chimera 32 | APASSSTKKTQLQLEHLLLDLQMILNGIQSMEIDAT |
| | (T3A, 192G, C125S) | TFKFYMPKKATELKHLQCLEEELKPLEEVLNLAQS |
| | | KNFHLRPRDLISNINVGVLELKGSETTFMCEYADET |
| | | ATIVEFLNRWITFSQSIISTLT |
| 74 | IL-2-IL-15 Chimera 33 | APTSSSTKKTQLQLEHLLLDLQMILNGIQSMEIDAT |
| | (192D, C125S) | TFKFYMPKKATELKHLQCLEEELKPLEEVLNLAQS |
| | | KNFHLRPRDLISNINVDVLELKGSETTFMCEYADET |
| | | ATIVEFLNRWITFSQSIISTLT |
| 75 | IL-2-IL-15 Chimera 34 | APASSSTKKTQLQLEHLLLDLQMILNGIQSMEIDAT |
| | (T3A, I92D, C125S) | TFKFYMPKKATELKHLQCLEEELKPLEEVLNLAQS |
| | | KNFHLRPRDLISNINVDVLELKGSETTFMCEYADET |
| | | ATIVEFLNRWITFSQSIISTLT |
| 76 | IL-2-IL-15 Chimera 35 | APTSSSTKKTQLQLEHLLLDLQMILNGIQSMEIDAT |
| | (192E, C125S) | TFKFYMPKKATELKHLQCLEEELKPLEEVLNLAQS |
| | | KNFHLRPRDLISNINVEVLELKGSETTFMCEYADET |
| | | ATIVEFLNRWITFSQSIISTLT |
| 77 | IL-2-IL-15 Chimera 36 | APASSSTKKTQLQLEHLLLDLQMILNGIQSMEIDAT |
| | (T3A, I92E, C125S) | TFKFYMPKKATELKHLQCLEEELKPLEEVLNLAQS |
| | | KNFHLRPRDLISNINVEVLELKGSETTFMCEYADET |
| | | ATIVEFLNRWITFSQSIISTLT |
| 78 | IL-2-IL-15 Chimera 37 | APTSSSTKKTQLQLEELLLDLQMILNGIQSMEIDAT |
| | (H16E, I92A, C125S) | TFKFYMPKKATELKHLQCLEEELKPLEEVLNLAQS |
| | | KNFHLRPRDLISNINVAVLELKGSETTFMCEYADET |
| | | ATIVEFLNRWITFSQSIISTLT |
| 79 | IL-2-IL-15 Chimera 38 | APASSSTKKTQLQLEELLLDLQMILNGIQSMEIDAT |
| | (T3A, H16E, 192A, C125S) | TFKFYMPKKATELKHLQCLEEELKPLEEVLNLAQS |
| | | KNFHLRPRDLISNINVAVLELKGSETTFMCEYADET |
| | | ATIVEFLNRWITFSQSIISTLT |
| 80 | IL-2-IL-15 Chimera 39 | APTSSSTKKTQLQLKHLLLDLQMILNGIQSMEIDAT |
| | (E15K, I92A, C125S) | TFKFYMPKKATELKHLQCLEEELKPLEEVLNLAQS |
| | | KNFHLRPRDLISNINVAVLELKGSETTFMCEYADET |
| | | ATIVEFLNRWITFSQSIISTLT |
| 81 | IL-2-IL-15 Chimera 40 | APASSSTKKTQLQLKHLLLDLQMILNGIQSMEIDAT |
| | (T3A, E15K, 192A, C125S) | TFKFYMPKKATELKHLQCLEEELKPLEEVLNLAQS |
| | | KNFHLRPRDLISNINVAVLELKGSETTFMCEYADET |
| | | ATIVEFLNRWITFSQSIISTLT |
| 82 | Mouse IL-2-IL-15 Chimera | APTSSSTSSSTAEAQQQQQQQQQQQQHLEQLLMDL |
| | | QELLSRMQSMEIDATTFKFYLPKQATELKDLQCLE |
| | | DELGPLRHVLDLTQSKSFQLEDAENFISNIRVTVVK |
| | | LKGSDNTFECQFDDESATVVDFLRRWIAFSQSIISTS |
| | | PQ |
| 83 | Anti-HSA-1 CDR1 | GSTWSINT |
| 84 | Anti-HSA-1 CDR2 | ISSGGST |
| 85 | Anti-HSA-1 CDR3 | YAQSTWYPPS |
| 86 | Anti-HSA-1 FR1 | EVQLVESGGGLVQPGGSLRLSCAAS |
| 87 | Anti-HSA-1 FR2 | LAWYRQAPGKQRDLVAR |
| 88 | Anti-HSA-1 FR3 | YYADSVKGRFTISRDNSKNTLYLQMNSLRAEDTAV |
| | | YYC |
| 89 | Anti-HSA-1 FR4 | WGQGTLVTVSS |
| 90 | Anti-HSA-1 | EVQLVESGGGLVQPGGSLRLSCAASGSTWSINTLA |
| | | WYRQAPGKQRDLVARISSGGSTYYADSVKGRFTIS |
| | | RDNSKNTLYLQMNSLRAEDTAVYYCYAQSTWYPP |
| | | SWGQGTLVTVSS |
| 91 | Anti-HSA-2 CDR1 | GFAFRGFG |
| 92 | Anti-HSA-2 CDR2 | INNGGSDT |
| 93 | Anti-HSA-2 CDR3 | AIGGPGASP |
| 94 | Anti-HSA-2 FR1 | QVQLVESGGGVVQPGGSLRLSCAAS |
| 95 | Anti-HSA-2 FR2 | MSWVRQAPGKGLEWVSS |
| 96 | Anti-HSA-2 FR4 | SGQGTQVTVSS |
| 97 | Anti-HSA-2 | QVQLVESGGGVVQPGGSLRLSCAASGFAFRGFGM |
| | | SWVRQAPGKGLEWVSSINNGGSDTYYADSVKGRF |
| | | TISRDNSKNTLYLQMNSLRAEDTAVYYCAIGGPGA |
| | | SPSGQGTQVTVSS |
| 98 | hIgG1 C_{H} | ASTKGPSVFPLAPSSKSTSGGTAALGCLVKDYFPEP |
| | | VTVSWNSGALTSGVHTFPAVLQSSGLYSLSSVVTV |
| | | PSSSLGTQTYICNVNHKPSNTKVDKKVEPKSCDKT |
| | | HTCPPCPAPELLGGPSVFLFPPKPKDTLMISRTPEVT |
| | | CVVVDVSHEDPEVKFNWYVDGVEVHNAKTKPRE |
| | | EQYNSTYRVVSVLTVLHQDWLNGKEYKCKVSNK |
| | | ALPAPIEKTISKAKGQPREPQVYTLPPSREEMTKNQ |
| | | VSLTCLVKGFYPSDIAVEWESNGQPENNYKTTPPV |
| | | LDSDGSFFLYSKLTVDKSRWQQGNVFSCSVMHEA |
| | | LHNHYTQKSLSLSPGK |
| 99 | hIgG1 C_{H} Mutein | ASTKGPSVFPLAPSSKSTSGGTAALGCLVKDYFPEP |
| | (N297A) | VTVSWNSGALTSGVHTFPAVLQSSGLYSLSSVVTV |
| | | PSSSLGTQTYICNVNHKPSNTKVDKKVEPKSCDKT |
| | | HTCPPCPAPELLGGPSVFLFPPKPKDTLMISRTPEVT |
| | | CVVVDVSHEDPEVKFNWYVDGVEVHNAKTKPRE |
| | | EQYASTYRVVSVLTVLHQDWLNGKEYKCKVSNK |
| | | ALPAPIEKTISKAKGQPREPQVYTLPPSREEMTKNQ |
| | | VSLTCLVKGFYPSDIAVEWESNGQPENNYKTTPPV |
| | | LDSDGSFFLYSKLTVDKSRWQQGNVFSCSVMHEA |
| | | LHNHYTQKSLSLSPGK |
| 100 | hIgG1 C_{H} Mutein | ASTKGPSVFPLAPSSKSTSGGTAALGCLVKDYFPEP |
| | (LALA, N297A) | VTVSWNSGALTSGVHTFPAVLQSSGLYSLSSVVTV |
| | | PSSSLGTQTYICNVNHKPSNTKVDKKVEPKSCDKT |
| | | HTCPPCPAPEAAGGPSVFLFPPKPKDTLMISRTPEVT |
| | | CVVVDVSHEDPEVKFNWYVDGVEVHNAKTKPRE |
| | | EQYASTYRVVSVLTVLHQDWLNGKEYKCKVSNK |
| | | ALPAPIEKTISKAKGQPREPQVYTLPPSREEMTKNQ |
| | | VSLTCLVKGFYPSDIAVEWESNGQPENNYKTTPPV |
| | | LDSDGSFFLYSKLTVDKSRWQQGNVFSCSVMHEA |
| | | LHNHYTQKSLSLSPGK |
| 101 | hIgG1 C_{H} Mutein | ASTKGPSVFPLAPSSKSTSGGTAALGCLVKDYFPEP |
| | (S354C, T366W) | VTVSWNSGALTSGVHTFPAVLQSSGLYSLSSVVTV |
| | | PSSSLGTQTYICNVNHKPSNTKVDKKVEPKSCDKT |
| | | HTCPPCPAPELLGGPSVFLFPPKPKDTLMISRTPEVT |
| | | CVVVDVSHEDPEVKFNWYVDGVEVHNAKTKPRE |
| | | EQYNSTYRVVSVLTVLHQDWLNGKEYKCKVSNK |
| | | ALPAPIEKTISKAKGQPREPQVYTLPPCREEMTKNQ |
| | | VSLWCLVKGFYPSDIAVEWESNGQPENNYKTTPPV |
| | | LDSDGSFFLYSKLTVDKSRWQQGNVFSCSVMHEA |
| | | LHNHYTQKSLSLSPGK |
| 102 | hIgG1 C_{H} Mutein | ASTKGPSVFPLAPSSKSTSGGTAALGCLVKDYFPEP |
| | (Y349C, T366S, L368A, | VTVSWNSGALTSGVHTFPAVLQSSGLYSLSSVVTV |
| | Y407V) | PSSSLGTQTYICNVNHKPSNTKVDKKVEPKSCDKT |
| | | HTCPPCPAPELLGGPSVFLFPPKPKDTLMISRTPEVT |
| | | CVVVDVSHEDPEVKFNWYVDGVEVHNAKTKPRE |
| | | EQYNSTYRVVSVLTVLHQDWLNGKEYKCKVSNK |
| | | ALPAPIEKTISKAKGQPREPQVCTLPPSREEMTKNQ |
| | | VSLSCAVKGFYPSDIAVEWESNGQPENNYKTTPPV |
| | | LDSDGSFFLVSKLTVDKSRWQQGNVFSCSVMHEA |
| | | LHNHYTQKSLSLSPGK |
| 103 | hIgG1 C_{H} Mutein | ASTKGPSVFPLAPSSKSTSGGTAALGCLVKDYFPEP |
| | (S354C, T366W, N297A) | VTVSWNSGALTSGVHTFPAVLQSSGLYSLSSVVTV |
| | | PSSSLGTQTYICNVNHKPSNTKVDKKVEPKSCDKT |
| | | HTCPPCPAPELLGGPSVFLFPPKPKDTLMISRTPEVT |
| | | CVVVDVSHEDPEVKFNWYVDGVEVHNAKTKPRE |
| | | EQYASTYRVVSVLTVLHQDWLNGKEYKCKVSNK |
| | | ALPAPIEKTISKAKGQPREPQVYTLPPCREEMTKNQ |
| | | VSLWCLVKGFYPSDIAVEWESNGQPENNYKTTPPV |
| | | LDSDGSFFLYSKLTVDKSRWQQGNVFSCSVMHEA |
| | | LHNHYTQKSLSLSPGK |
| 104 | hIgG1 C_{H} Mutein | ASTKGPSVFPLAPSSKSTSGGTAALGCLVKDYFPEP |
| | (Y349C, T366S, L368A, | VTVSWNSGALTSGVHTFPAVLQSSGLYSLSSVVTV |
| | Y407V, N297A) | PSSSLGTQTYICNVNHKPSNTKVDKKVEPKSCDKT |
| | | HTCPPCPAPELLGGPSVFLFPPKPKDTLMISRTPEVT |
| | | CVVVDVSHEDPEVKFNWYVDGVEVHNAKTKPRE |
| | | EQYASTYRVVSVLTVLHQDWLNGKEYKCKVSNK |
| | | ALPAPIEKTISKAKGQPREPQVCTLPPSREEMTKNQ |
| | | VSLSCAVKGFYPSDIAVEWESNGQPENNYKTTPPV |
| | | LDSDGSFFLVSKLTVDKSRWQQGNVFSCSVMHEA |
| | | LHNHYTQKSLSLSPGK |
| 105 | hIgG1 C_{H} Mutein | ASTKGPSVFPLAPSSKSTSGGTAALGCLVKDYFPEP |
| | (S354C, T366W, LALA, | VTVSWNSGALTSGVHTFPAVLQSSGLYSLSSVVTV |
| | N297A) | PSSSLGTQTYICNVNHKPSNTKVDKKVEPKSCDKT |
| | | HTCPPCPAPEAAGGPSVFLFPPKPKDTLMISRTPEVT |
| | | CVVVDVSHEDPEVKFNWYVDGVEVHNAKTKPRE |
| | | EQYASTYRVVSVLTVLHQDWLNGKEYKCKVSNK |
| | | ALPAPIEKTISKAKGQPREPQVYTLPPCREEMTKNQ |
| | | VSLWCLVKGFYPSDIAVEWESNGQPENNYKTTPPV |
| | | LDSDGSFFLYSKLTVDKSRWQQGNVFSCSVMHEA |
| | | LHNHYTQKSLSLSPGK |
| 106 | hIgG1 C_{H} Mutein | ASTKGPSVFPLAPSSKSTSGGTAALGCLVKDYFPEP |
| | (Y349C, T366S, L368A, | VTVSWNSGALTSGVHTFPAVLQSSGLYSLSSVVTV |
| | Y407V, LALA, N297A) | PSSSLGTQTYICNVNHKPSNTKVDKKVEPKSCDKT |
| | | HTCPPCPAPEAAGGPSVFLFPPKPKDTLMISRTPEVT |
| | | CVVVDVSHEDPEVKFNWYVDGVEVHNAKTKPRE |
| | | EQYASTYRVVSVLTVLHQDWLNGKEYKCKVSNK |
| | | ALPAPIEKTISKAKGQPREPQVCTLPPSREEMTKNQ |
| | | VSLSCAVKGFYPSDIAVEWESNGQPENNYKTTPPV |
| | | LDSDGSFFLVSKLTVDKSRWQQGNVFSCSVMHEA |
| | | LHNHYTQKSLSLSPGK |
| 107 | hIgG4 C_{H} | ASTKGPSVFPLAPCSRSTSESTAALGCLVKDYFPEP |
| | | VTVSWNSGALTSGVHTFPAVLQSSGLYSLSSVVTV |
| | | PSSSLGTKTYTCNVDHKPSNTKVDKRVESKYGPPC |
| | | PPCPAPEFLGGPSVFLFPPKPKDTLMISRTPEVTCVV |
| | | VDVSQEDPEVQFNWYVDGVEVHNAKTKPREEQFN |
| | | STYRVVSVLTVLHQDWLNGKEYKCKVSNKGLPSSI |
| | | EKTISKAKGQPREPQVYTLPPSQEEMTKNQVSLTCL |
| | | VKGFYPSDIAVEWESNGQPENNYKTTPPVLDSDGS |
| | | FFLYSRLTVDKSRWQEGNVFSCSVMHEALHNHYT |
| | | QKSLSLSLGK |
| 108 | hIgG4 C_{H} Mutein | ASTKGPSVFPLAPCSRSTSESTAALGCLVKDYFPEP |
| | (T366W) | VTVSWNSGALTSGVHTFPAVLQSSGLYSLSSVVTV |
| | | PSSSLGTKTYTCNVDHKPSNTKVDKRVESKYGPPC |
| | | PPCPAPEFLGGPSVFLFPPKPKDTLMISRTPEVTCVV |
| | | VDVSQEDPEVQFNWYVDGVEVHNAKTKPREEQFN |
| | | STYRVVSVLTVLHQDWLNGKEYKCKVSNKGLPSSI |
| | | EKTISKAKGQPREPQVYTLPPSQEEMTKNQVSLWC |
| | | LVKGFYPSDIAVEWESNGQPENNYKTTPPVLDSDG |
| | | SFFLYSRLTVDKSRWQEGNVFSCSVMHEALHNHY |
| | | TQKSLSLSLGK |
| 109 | hIgG4 C_{H} Mutein | ASTKGPSVFPLAPCSRSTSESTAALGCLVKDYFPEP |
| | (T366S, T368A) | VTVSWNSGALTSGVHTFPAVLQSSGLYSLSSVVTV |
| | | PSSSLGTKTYTCNVDHKPSNTKVDKRVESKYGPPC |
| | | PPCPAPEFLGGPSVFLFPPKPKDTLMISRTPEVTCVV |
| | | VDVSQEDPEVQFNWYVDGVEVHNAKTKPREEQFN |
| | | STYRVVSVLTVLHQDWLNGKEYKCKVSNKGLPSSI |
| | | EKTISKAKGQPREPQVYTLPPSQEEMTKNQVSLSC |
| | | AVKGFYPSDIAVEWESNGQPENNYKTTPPVLDSDG |
| | | SFFLVSRLTVDKSRWQEGNVFSCSVMHEALHNHY |
| | | TQKSLSLSLGK |
| 110 | hKappa C_{L} | RTVAAPSVFIFPPSDEQLKSGTASVVCLLNNFYPRE |
| | | AKVQWKVDNALQSGNSQESVTEQDSKDSTYSLSS |
| | | TLTLSKADYEKHKVYACEVTHQGLSSPVTKSFNRG |
| | | EC |
| 111 | Anti-hPD-1-A CDRL1 | QSVSSY |
| | (IMGT) | |
| | Anti-hPD-1-A CDRL2 | DAS |
| | (IMGT) | |
| 112 | Anti-hPD-1-A CDRL3 | QQSSNWPRT |
| | (IMGT) | |
| 113 | Anti-hPD-1-A CDRH1 | GITFSNSG |
| | (IMGT) | |
| 114 | Anti-hPD-1-A CDRH2 | IWYDGSKR |
| | (IMGT) | |
| 115 | Anti-hPD-1-A CDRH3 | ATNDDY |
| | (IMGT) | |
| 116 | Anti-hPD-1-A Light Chain | EIVLTQSPATLSLSPGERATLSCRASQSVSSYLAWY |
| | Variable | QQKPGQAPRLLIYDASNRATGIPARFSGSGSGTDFT |
| | | LTISSLEPEDFAVYYCQQSSNWPRTFGQGTKVEIK |
| 117 | Anti-hPD-1-A Heavy Chain | QVQLVESGGGVVQPGRSLRLDCKASGITFSNSGMH |
| | Variable | WVRQAPGKGLEWVAVIWYDGSKRYYADSVKGRF |
| | | TISRDNSKNTLFLQMNSLRAEDTAVYYCATNDDY |
| | | WGQGTLVTVSS |
| 118 | Anti-hPD-1-A Light Chain | EIVLTQSPATLSLSPGERATLSCRASQSVSSYLAWY |
| | | QQKPGQAPRLLIYDASNRATGIPARFSGSGSGTDFT |
| | | LTISSLEPEDFAVYYCQQSSNWPRTFGQGTKVEIKR |
| | | TVAAPSVFIFPPSDEQLKSGTASVVCLLNNFYPREA |
| | | KVQWKVDNALQSGNSQESVTEQDSKDSTYSLSSTL |
| | | TLSKADYEKHKVYACEVTHQGLSSPVTKSFNRGEC |
| 119 | Anti-hPD-1-A Heavy Chain | QVQLVESGGGVVQPGRSLRLDCKASGITFSNSGMH |
| | | WVRQAPGKGLEWVAVIWYDGSKRYYADSVKGRF |
| | | TISRDNSKNTLFLQMNSLRAEDTAVYYCATNDDY |
| | | WGQGTLVTVSSASTKGPSVFPLAPCSRSTSESTAAL |
| | | GCLVKDYFPEPVTVSWNSGALTSGVHTFPAVLQSS |
| | | GLYSLSSVVTVPSSSLGTKTYTCNVDHKPSNTKVD |
| | | KRVESKYGPPCPPCPAPEFLGGPSVFLFPPKPKDTL |
| | | MISRTPEVTCVVVDVSQEDPEVQFNWYVDGVEVH |
| | | NAKTKPREEQFNSTYRVVSVLTVLHQDWLNGKEY |
| | | KCKVSNKGLPSSIEKTISKAKGQPREPQVYTLPPSQ |
| | | EEMTKNQVSLTCLVKGFYPSDIAVEWESNGQPENN |
| | | YKTTPPVLDSDGSFFLYSRLTVDKSRWQEGNVFSC |
| | | SVMHEALHNHYTQKSLSLSLGK |
| 120 | Anti-hPD-1-B CDRL1 | KGVSTSGYSY |
| | (IMGT) | |
| | Anti-hPD-1-B CDRL2 | LAS |
| | (IMGT) | |
| 121 | Anti-hPD-1-B CDRL3 | QHSRDLPLT |
| | (IMGT) | |
| 122 | Anti-hPD-1-B CDRH1 | GYTFTNYY |
| | (IMGT) | |
| 123 | Anti-hPD-1-B CDRH2 | INPSNGGT |
| | (IMGT) | |
| 124 | Anti-hPD-1-B CDRH3 | ARRDYRFDMGFDY |
| | (IMGT) | |
| 125 | Anti-hPD-1-B Light Chain | EIVLTQSPATLSLSPGERATLSCRASKGVSTSGYSYL |
| | Variable | HWYQQKPGQAPRLLIYLASYLESGVPARFSGSGSG |
| | | TDFTLTISSLEPEDFAVYYCQHSRDLPLTFGGGTKV |
| | | EIK |
| 126 | Anti-hPD-1-B Heavy Chain | QVQLVQSGVEVKKPGASVKVSCKASGYTFTNYYM |
| | Variable | YWVRQAPGQGLEWMGGINPSNGGTNFNEKFKNR |
| | | VTLTTDSSTTTAYMELKSLQFDDTAVYYCARRDY |
| | | RFDMGFDYWGQGTTVTVSS |
| 127 | Anti-hPD-1-B Light Chain | EIVLTQSPATLSLSPGERATLSCRASKGVSTSGYSYL |
| | | HWYQQKPGQAPRLLIYLASYLESGVPARFSGSGSG |
| | | TDFTLTISSLEPEDFAVYYCQHSRDLPLTFGGGTKV |
| | | EIKRTVAAPSVFIFPPSDEQLKSGTASVVCLLNNFYP |
| | | REAKVQWKVDNALQSGNSQESVTEQDSKDSTYSL |
| | | SSTLTLSKADYEKHKVYACEVTHQGLSSPVTKSFN |
| | | RGEC |
| 128 | Anti-hPD-1-B Heavy Chain | QVQLVQSGVEVKKPGASVKVSCKASGYTFTNYYM |
| | | YWVRQAPGQGLEWMGGINPSNGGTNFNEKFKNR |
| | | VTLTTDSSTTTAYMELKSLQFDDTAVYYCARRDY |
| | | RFDMGFDYWGQGTTVTVSSASTKGPSVFPLAPCSR |
| | | STSESTAALGCLVKDYFPEPVTVSWNSGALTSGVH |
| | | TFPAVLQSSGLYSLSSVVTVPSSSLGTKTYTCNVDH |
| | | KPSNTKVDKRVESKYGPPCPPCPAPEFLGGPSVFLF |
| | | PPKPKDTLMISRTPEVTCVVVDVSQEDPEVQFNWY |
| | | VDGVEVHNAKTKPREEQFNSTYRVVSVLTVLHQD |
| | | WLNGKEYKCKVSNKGLPSSIEKTISKAKGQPREPQ |
| | | VYTLPPSQEEMTKNQVSLTCLVKGFYPSDIAVEWE |
| | | SNGQPENNYKTTPPVLDSDGSFFLYSRLTVDKSRW |
| | | QEGNVFSCSVMHEALHNHYTQKSLSLSLGK |
| 129 | Anti-hPD-1-C CDRL1 | QSIVHSNGNTY |
| | (IMGT) | |
| | Anti-hPD-1-C CDRL2 | KVS |
| | (IMGT) | |
| 130 | Anti-hPD-1-C CDRL3 | FQGSHVPLT |
| | (IMGT) | |
| 131 | Anti-hPD-1-C CDRH1 | GYTFTDYE |
| | (IMGT) | |
| 132 | Anti-hPD-1-C CDRH2 | IESETGGT |
| | (IMGT) | |
| 133 | Anti-hPD-1-C CDRH3 | AREGITTVATTYYWYFDV |
| | (IMGT) | |
| 134 | Anti-hPD-1-C Light Chain | DVVMTQSPLSLPVTLGQPASISCRSSQSIVHSNGNT |
| | Variable | YLEWYLQKPGQSPQLLIYKVSNRFSGVPDRFSGSG |
| | | SGTDFTLKISRVEAEDVGVYYCFQGSHVPLTFGQG |
| | | TKLEIK |
| 135 | Anti-hPD-1-C Heavy Chain | QGQLVQSGAEVKKPGASVKVSCKASGYTFTDYEM |
| | Variable | HWVRQAPIHGLEWIGVIESETGGTAYNQKFKGRVT |
| | | ITADKSTSTAYMELSSLRSEDTAVYYCAREGITTVA |
| | | TTYYWYFDVWGQGTTVTVSS |
| 136 | Anti-hPD-1-C Light Chain | DVVMTQSPLSLPVTLGQPASISCRSSQSIVHSNGNT |
| | (Kappa) | YLEWYLQKPGQSPQLLIYKVSNRFSGVPDRFSGSG |
| | | SGTDFTLKISRVEAEDVGVYYCFQGSHVPLTFGQG |
| | | TKLEIKRTVAAPSVFIFPPSDEQLKSGTASVVCLLN |
| | | NFYPREAKVQWKVDNALQSGNSQESVTEQDSKDS |
| | | TYSLSSTLTLSKADYEKHKVYACEVTHQGLSSPVT |
| | | KSFNRGEC |
| 137 | Anti-hPD-1-C Heavy Chain | QGQLVQSGAEVKKPGASVKVSCKASGYTFTDYEM |
| | | HWVRQAPIHGLEWIGVIESETGGTAYNQKFKGRVT |
| | | ITADKSTSTAYMELSSLRSEDTAVYYCAREGITTVA |
| | | TTYYWYFDVWGQGTTVTVSSASTKGPSVFPLAPCS |
| | | RSTSESTAALGCLVKDYFPEPVTVSWNSGALTSGV |
| | | HTFPAVLQSSGLYSLSSVVTVPSSSLGTKTYTCNVD |
| | | HKPSNTKVDKRVESKYGPPCPPCPAPEFLGGPSVFL |
| | | FPPKPKDTLMISRTPEVTCVVVDVSQEDPEVQFNW |
| | | YVDGVEVHNAKTKPREEQFNSTYRVVSVLTVLHQ |
| | | DWLNGKEYKCKVSNKGLPSSIEKTISKAKGQPREP |
| | | QVYTLPPSQEEMTKNQVSLTCLVKGFYPSDIAVEW |
| | | ESNGQPENNYKTTPPVLDSDGSFFLYSRLTVDKSR |
| | | WQEGNVFSCSVMHEALHNHYTQKSLSLSLGK |
| 138 | Anti-hPD-1-D CDRL1 | QTIGTW |
| | (IMGT) | |
| | Anti-hPD-1-D CDRL2 | TAT |
| | (IMGT) | |
| 139 | Anti-hPD-1-D CDRL3 | QQVYSIPWT |
| | (IMGT) | |
| 140 | Anti-hPD-1-D CDRH1 | GFTFSSYM |
| | (IMGT) | |
| 141 | Anti-hPD-1-D CDRH2 | ISGGGANT |
| | (IMGT) | |
| 142 | Anti-hPD-1-D CDRH3 | ARQLYYFDY |
| | (IMGT) | |
| 143 | Anti-hPD-1-D Light Chain | DIQMTQSPSSLSASVGDRVTITCLASQTIGTWLTWY |
| | Variable | QQKPGKAPKLLIYTATSLADGVPSRFSGSGSGTDFT |
| | | LTISSLQPEDFATYYCQQVYSIPWTFGGGTKVEIK |
| 144 | Anti-hPD-1-D Heavy Chain | EVQLVESGGGLVQPGGSLRLSCAASGFTFSSYMMS |
| | Variable | WVRQAPGKGLEWVATISGGGANTYYPDSVKGRFT |
| | | ISRDNAKNSLYLQMNSLRAEDTAVYYCARQLYYF |
| | | DYWGQGTTVTVSS |
| 145 | Anti-hPD-1-D Light Chain | DIQMTQSPSSLSASVGDRVTITCLASQTIGTWLTWY |
| | | QQKPGKAPKLLIYTATSLADGVPSRFSGSGSGTDFT |
| | | LTISSLQPEDFATYYCQQVYSIPWTFGGGTKVEIKR |
| | | TVAAPSVFIFPPSDEQLKSGTASVVCLLNNFYPREA |
| | | KVQWKVDNALQSGNSQESVTEQDSKDSTYSLSSTL |
| | | TLSKADYEKHKVYACEVTHQGLSSPVTKSFNRGEC |
| 146 | Anti-hPD-1-D Heavy Chain | EVQLVESGGGLVQPGGSLRLSCAASGFTFSSYMMS |
| | | WVRQAPGKGLEWVATISGGGANTYYPDSVKGRFT |
| | | ISRDNAKNSLYLQMNSLRAEDTAVYYCARQLYYF |
| | | DYWGQGTTVTVSSASTKGPSVFPLAPCSRSTSESTA |
| | | ALGCLVKDYFPEPVTVSWNSGALTSGVHTFPAVLQ |
| | | SSGLYSLSSVVTVPSSSLGTKTYTCNVDHKPSNTKV |
| | | DKRVESKYGPPCPPCPAPEFLGGPSVFLFPPKPKDT |
| | | LMISRTPEVTCVVVDVSQEDPEVQFNWYVDGVEV |
| | | HNAKTKPREEQFNSTYRVVSVLTVLHQDWLNGKE |
| | | YKCKVSNKGLPSSIEKTISKAKGQPREPQVYTLPPS |
| | | QEEMTKNQVSLTCLVKGFYPSDIAVEWESNGQPEN |
| | | NYKTTPPVLDSDGSFFLYSRLTVDKSRWQEGNVFS |
| | | CSVMHEALHNHYTQKSLSLSLGK |
| 147 | Anti-hPD-1-E CDRL1 | QGISSW |
| | (IMGT) | |
| | Anti-hPD-1-E CDRL2 | AAS |
| | (IMGT) | |
| 148 | Anti-hPD-1-E CDRL3 | QQANHLPFT |
| | (IMGT) | |
| 149 | Anti-hPD-1-E CDRH1 | GGTFSSYA |
| | (IMGT) | |
| 150 | Anti-hPD-1-E CDRH2 | IIPMFDTA |
| | (IMGT) | |
| 151 | Anti-hPD-1-E CDRH3 | ARAEHSSTGTFDY |
| | (IMGT) | |
| 152 | Anti-hPD-1-E Light Chain | DIQMTQSPSSVSASVGDRVTITCRASQGISSWLAW |
| | Variable | YQQKPGKAPKLLISAASSLQSGVPSRFSGSGSGTDF |
| | | TLTISSLQPEDFATYYCQQANHLPFTFGGGTKVEIK |
| 153 | Anti-hPD-1-E Heavy Chain | QVQLVQSGAEVKKPGSSVKVSCKASGGTFSSYAIS |
| | Variable | WVRQAPGQGLEWMGLIIPMFDTAGYAQKFQGRV |
| | | AITVDESTSTAYMELSSLRSEDTAVYYCARAEHSST |
| | | GTFDYWGQGTLVTVSS |
| 154 | Anti-hPD-1-E Light Chain | DIQMTQSPSSVSASVGDRVTITCRASQGISSWLAW |
| | | YQQKPGKAPKLLISAASSLQSGVPSRFSGSGSGTDF |
| | | TLTISSLQPEDFATYYCQQANHLPFTFGGGTKVEIK |
| | | RTVAAPSVFIFPPSDEQLKSGTASVVCLLNNFYPRE |
| | | AKVQWKVDNALQSGNSQESVTEQDSKDSTYSLSS |
| | | TLTLSKADYEKHKVYACEVTHQGLSSPVTKSFNRG |
| | | EC |
| 155 | Anti-hPD-1-E Heavy Chain | QVQLVQSGAEVKKPGSSVKVSCKASGGTFSSYAIS |
| | | WVRQAPGQGLEWMGLIIPMEDTAGYAQKFQGRV |
| | | AITVDESTSTAYMELSSLRSEDTAVYYCARAEHSST |
| | | GTFDYWGQGTLVTVSSASTKGPSVFPLAPCSRSTSE |
| | | STAALGCLVKDYFPEPVTVSWNSGALTSGVHTFPA |
| | | VLQSSGLYSLSSVVTVPSSSLGTKTYTCNVDHKPSN |
| | | TKVDKRVESKYGPPCPPCPAPEFLGGPSVFLFPPKP |
| | | KDTLMISRTPEVTCVVVDVSQEDPEVQFNWYVDG |
| | | VEVHNAKTKPREEQFNSTYRVVSVLTVLHQDWLN |
| | | GKEYKCKVSNKGLPSSIEKTISKAKGQPREPQVYTL |
| | | PPSQEEMTKNQVSLTCLVKGFYPSDIAVEWESNGQ |
| | | PENNYKTTPPVLDSDGSFFLYSRLTVDKSRWQEGN |
| | | VFSCSVMHEALHNHYTQKSLSLSLGK |
| 156 | Anti-hPD-1-F CDRL1 | QSLLDSGNQKNF |
| | (IMGT) | |
| | Anti-hPD-1-F CDRL2 | WAS |
| | (IMGT) | |
| 157 | Anti-hPD-1-F CDRL3 | QNDYSYPYT |
| | (IMGT) | |
| 158 | Anti-hPD-1-F CDRH1 | GYTFTTYW |
| | (IMGT) | |
| 159 | Anti-hPD-1-F CDRH2 | IYPGTGGS |
| | (IMGT) | |
| 160 | Anti-hPD-1-F CDRH3 | TRWTTGTGAY |
| | (IMGT) | |
| 161 | Anti-hPD-1-F Light Chain | EIVLTQSPATLSLSPGERATLSCKSSQSLLDSGNQK |
| | Variable | NFLTWYQQKPGQAPRLLIYWASTRESGVPSRFSGS |
| | | GSGTDFTETISSLEAEDAATYYCQNDYSYPYTFGQ |
| | | GTKVEIK |
| 162 | Anti-hPD-1-F Heavy Chain | EVQLVQSGAEVKKPGESLRISCKGSGYTFTTYWMH |
| | Variable | WVRQATGQGLEWMGNIYPGTGGSNFDEKEKNRV |
| | | TITADKSTSTAYMELSSLRSEDTAVYYCTRWTTGT |
| | | GAYWGQGTTVTVSS |
| 163 | Anti-hPD-1-F Light Chain | EIVLTQSPATLSLSPGERATLSCKSSQSLLDSGNQK |
| | | NFLTWYQQKPGQAPRLLIYWASTRESGVPSRFSGS |
| | | GSGTDFTETISSLEAEDAATYYCQNDYSYPYTFGQ |
| | | GTKVEIKRTVAAPSVFIFPPSDEQLKSGTASVVCLL |
| | | NNFYPREAKVQWKVDNALQSGNSQESVTEQDSKD |
| | | STYSLSSTLTLSKADYEKHKVYACEVTHQGLSSPV |
| | | TKSFNRGEC |
| 164 | Anti-hPD-1-F Heavy Chain | EVQLVQSGAEVKKPGESLRISCKGSGYTFTTYWMH |
| | | WVRQATGQGLEWMGNIYPGTGGSNFDEKEKNRV |
| | | TITADKSTSTAYMELSSLRSEDTAVYYCTRWTTGT |
| | | GAYWGQGTTVTVSSASTKGPSVFPLAPCSRSTSEST |
| | | AALGCLVKDYFPEPVTVSWNSGALTSGVHTFPAVL |
| | | QSSGLYSLSSVVTVPSSSLGTKTYTCNVDHKPSNTK |
| | | VDKRVESKYGPPCPPCPAPEFLGGPSVFLFPPKPKD |
| | | TLMISRTPEVTCVVVDVSQEDPEVQFNWYVDGVE |
| | | VHNAKTKPREEQFNSTYRVVSVLTVLHQDWLNGK |
| | | EYKCKVSNKGLPSSIEKTISKAKGQPREPQVYTLPP |
| | | SQEEMTKNQVSLTCLVKGFYPSDIAVEWESNGQPE |
| | | NNYKTTPPVLDSDGSFFLYSRLTVDKSRWQEGNVF |
| | | SCSVMHEALHNHYTQKSLSLSLQG |
| 165 | Anti-hPD-1-G CDRL1 | ESVSND |
| | (IMGT) | |
| | Anti-hPD-1-G CDRL2 | YAF |
| | (IMGT) | |
| 166 | Anti-hPD-1-G CDRL3 | HQAYSSPYT |
| | (IMGT) | |
| 167 | Anti-hPD-1-G CDRH1 | GFSLTSYG |
| | (IMGT) | |
| 168 | Anti-hPD-1-G CDRH2 | IYADGST |
| | (IMGT) | |
| 169 | Anti-hPD-1-G CDRH3 | ARAYGNYWYIDV |
| | (IMGT) | |
| 170 | Anti-hPD-1-G Light Chain | DIVMTQSPDSLAVSLGERATINCKSSESVSNDVAW |
| | Variable | YQQKPGQPPKLLINYAFHRFTGVPDRFSGSGYGTD |
| | | FTLTISSLQAEDVAVYYCHQAYSSPYTFGQGTKLEI |
| | | K |
| 171 | Anti-hPD-1-G Heavy Chain | QVQLQESGPGLVKPSETLSLTCTVSGFSLTSYGVH |
| | Variable | WIRQPPGKGLEWIGVIYADGSTNYNPSLKSRVTISK |
| | | DTSKNQVSLKLSSVTAADTAVYYCARAYGNYWYI |
| | | DVWGQGTTVTVSS |
| 172 | Anti-hPD-1-G Light Chain | DIVMTQSPDSLAVSLGERATINCKSSESVSNDVAW |
| | | YQQKPGQPPKLLINYAFHRFTGVPDRFSGSGYGTD |
| | | FTLTISSLQAEDVAVYYCHQAYSSPYTFGQGTKLEI |
| | | KRTVAAPSVFIFPPSDEQLKSGTASVVCLLNNFYPR |
| | | EAKVQWKVDNALQSGNSQESVTEQDSKDSTYSLS |
| | | STLTLSKADYEKHKVYACEVTHQGLSSPVTKSFNR |
| | | GEC |
| 173 | Anti-hPD-1-G Heavy Chain | QVQLQESGPGLVKPSETLSLTCTVSGFSLTSYGVH |
| | | WIRQPPGKGLEWIGVIYADGSTNYNPSLKSRVTISK |
| | | DTSKNQVSLKLSSVTAADTAVYYCARAYGNYWYI |
| | | DVWGQGTTVTVSSASTKGPSVFPLAPCSRSTSESTA |
| | | ALGCLVKDYFPEPVTVSWNSGALTSGVHTFPAVLQ |
| | | SSGLYSLSSVVTVPSSSLGTKTYTCNVDHKPSNTKV |
| | | DKRVESKYGPPCPPCPAPPVAGGPSVFLFPPKPKDT |
| | | LMISRTPEVTCVVVAVSQEDPEVQENWYVDGVEV |
| | | HNAKTKPREEQENSTYRVVSVLTVVHQDWLNGKE |
| | | YKCKVSNKGLPSSIEKTISKAKGQPREPQVYTLPPS |
| | | QEEMTKNQVSLTCLVKGFYPSDIAVEWESNGQPEN |
| | | NYKTTPPVLDSDGSFFLYSKLTVDKSRWQEGNVFS |
| | | CSVMHEALHNHYTQKSLSLSLGIK |
| 174 | Anti-hPD-1-H CDRL1 | LSINTF |
| | (IMGT) | |
| | Anti-hPD-1-H CDRL2 | AAS |
| | (IMGT) | |
| 175 | Anti-hPD-1-H CDRL3 | QQSSNTPFT |
| | (IMGT) | |
| 176 | Anti-hPD-1-H CDRH1 | GFTFSNFG |
| | (IMGT) | |
| 177 | Anti-hPD-1-H CDRH2 | ISGGGRDT |
| | (IMGT) | |
| 178 | Anti-hPD-1-H CDRH3 | VKWGNIYFDY |
| | (IMGT) | |
| 179 | Anti-hPD-1-H Light Chain | DIQMTQSPSSLSASVGDSITITCRASLSINTFLNWYQ |
| | Variable | QKPGKAPNLLIYAASSLHGGVPSRFSGSGSGTDFTL |
| | | TIRTLQPEDFATYYCQQSSNTPFTFGPGTVVDFR |
| 180 | Anti-hPD-1-H Heavy Chain | EVQLLESGGVLVQPGGSLRLSCAASGFTFSNFGMT |
| | Variable | WVRQAPGKGLEWVSGISGGGRDTYFADSVKGRFT |
| | | ISRDNSKNTLYLQMNSLKGEDTAVYYCVKWGNIY |
| | | FDYWGQGTLVTVSS |
| 181 | Anti-hPD-1-H Light Chain | DIQMTQSPSSLSASVGDSITITCRASLSINTFLNWYQ |
| | | QKPGKAPNLLIYAASSLHGGVPSRFSGSGSGTDFTL |
| | | TIRTLQPEDFATYYCQQSSNTPFTFGPGTVVDFRRT |
| | | VAAPSVFIFPPSDEQLKSGTASVVCLLNNFYPREAK |
| | | VQWKVDNALQSGNSQESVTEQDSKDSTYSLSSTLT |
| | | LSKADYEKHKVYACEVTHQGLSSPVTKSFNRGEC |
| 182 | Anti-hPD-1-H Heavy Chain | EVQLLESGGVLVQPGGSLRLSCAASGFTFSNFGMT |
| | | WVRQAPGKGLEWVSGISGGGRDTYFADSVKGRFT |
| | | ISRDNSKNTLYLQMNSLKGEDTAVYYCVKWGNIY |
| | | FDYWGQGTLVTVSSASTKGPSVFPLAPCSRSTSEST |
| | | AALGCLVKDYFPEPVTVSWNSGALTSGVHTFPAVL |
| | | QSSGLYSLSSVVTVPSSSLGTKTYTCNVDHKPSNTK |
| | | VDKRVESKYGPPCPPCPAPEFLGGPSVFLFPPKPKD |
| | | TLMISRTPEVTCVVVDVSQEDPEVQFNWYVDGVE |
| | | VHNAKTKPREEQFNSTYRVVSVLTVLHQDWLNGK |
| | | EYKCKVSNKGLPSSIEKTISKAKGQPREPQVYTLPP |
| | | SQEEMTKNQVSLTCLVKGFYPSDIAVEWESNGQPE |
| | | NNYKTTPPVLDSDGSFFLYSRLTVDKSRWQEGNVF |
| | | SCSVMHEALHNHYTQKSLSLSLGK |
| 183 | Anti-hPD-1-I CDRL1 | QDVTTA |
| | (IMGT) | |
| | Anti-hPD-1-I CDRL2 | WAS |
| | (IMGT) | |
| 184 | Anti-hPD-1-I CDRL3 | QQHYTIPWT |
| | (IMGT) | |
| 185 | Anti-hPD-1-I CDRH1 | GFTFSNYG |
| | (IMGT) | |
| 186 | Anti-hPD-1-I CDRH2 | ISGGGSNI |
| | (IMGT) | |
| 187 | Anti-hPD-1-I CDRH3 | VSYYYGIDF |
| | (IMGT) | |
| 188 | Anti-hPD-1-I Light Chain | DIQMTQSPSSLSASVGDRVTITCKASQDVTTAVAW |
| | Variable | YQQKPGKAPKLLIYWASTRHTGVPSRFSGSGSGTD |
| | | FTLTISSLQPEDFATYYCQQHYTIPWTFGGGTKLEI |
| | | K |
| 189 | Anti-hPD-1-I Heavy Chain | QVQLVESGGGLVKPGGSLRLSCAASGFTFSNYGMS |
| | Variable | WIRQAPGKGLEWVSTISGGGSNIYYADSVKGRFTIS |
| | | RDNAKNSLYLQMNSLRAEDTAVYYCVSYYYGIDF |
| | | WGQGTSVTVSS |
| 190 | Anti-hPD-1-I Light Chain | DIQMTQSPSSLSASVGDRVTITCKASQDVTTAVAW |
| | | YQQKPGKAPKLLIYWASTRHTGVPSRFSGSGSGTD |
| | | FTLTISSLQPEDFATYYCQQHYTIPWTFGGGTKLEI |
| | | KRTVAAPSVFIFPPSDEQLKSGTASVVCLLNNFYPR |
| | | EAKVQWKVDNALQSGNSQESVTEQDSKDSTYSLS |
| | | STLTLSKADYEKHKVYACEVTHQGLSSPVTKSFNR |
| | | GEC |
| 191 | Anti-hPD-1-I Heavy Chain | QVQLVESGGGLVKPGGSLRLSCAASGFTFSNYGMS |
| | | WIRQAPGKGLEWVSTISGGGSNIYYADSVKGRFTIS |
| | | RDNAKNSLYLQMNSLRAEDTAVYYCVSYYYGIDF |
| | | WGQGTSVTVSSASTKGPSVFPLAPCSRSTSESTAAL |
| | | GCLVKDYFPEPVTVSWNSGALTSGVHTFPAVLQSS |
| | | GLYSLSSVVTVPSSSLGTKTYTCNVDHKPSNTKVD |
| | | KRVESKYGPPCPPCPAPEFLGGPSVFLFPPKPKDTL |
| | | MISRTPEVTCVVVDVSQEDPEVQFNWYVDGVEVH |
| | | NAKTKPREEQFNSTYRVVSVLTVLHQDWLNGKEY |
| | | KCKVSNKGLPSSIEKTISKAKGQPREPQVYTLPPSQ |
| | | EEMTKNQVSLTCLVKGFYPSDIAVEWESNGQPENN |
| | | YKTTPPVLDSDGSFFLYSRLTVDKSRWQEGNVFSC |
| | | SVMHEALHNHYTQKSLSLSLGK |
| 192 | Anti-hPD-1-J CDRL1 | ESVDNYGMSF |
| | (IMGT) | |
| | Anti-hPD-1-J CDRL2 | AAS |
| | (IMGT) | |
| 193 | Anti-hPD-1-J CDRL3 | QQSKEVPYT |
| | (IMGT) | |
| 194 | Anti-hPD-1-J CDRH1 | GYSFTSYW |
| | (IMGT) | |
| 195 | Anti-hPD-1-J CDRH2 | IHPSDSET |
| | (IMGT) | |
| 196 | Anti-hPD-1-J CDRH3 | AREHYGTSPFAY |
| | (IMGT) | |
| 197 | Anti-hPD-1-J Light Chain | EIVLTQSPATLSLSPGERATLSCRASESVDNYGMSF |
| | Variable | MNWFQQKPGQPPKLLIHAASNQGSGVPSRFSGSGS |
| | | GTDFTLTISSLEPEDFAVYFCQQSKEVPYTFGGGTK |
| | | VEIK |
| 198 | Anti-hPD-1-J Heavy Chain | QVQLVQSGAEVKKPGASVKVSCKASGYSFTSYWM |
| | Variable | NWVRQAPGQGLEWIGVIHPSDSETWLDQKFKDRV |
| | | TITVDKSTSTAYMELSSLRSEDTAVYYCAREHYGT |
| | | SPFAYWGQGTLVTVSS |
| 199 | Anti-hPD-1-J Light Chain | EIVLTQSPATLSLSPGERATLSCRASESVDNYGMSF |
| | | MNWFQQKPGQPPKLLIHAASNQGSGVPSRFSGSGS |
| | | GTDFTLTISSLEPEDFAVYFCQQSKEVPYTFGGGTK |
| | | VEIKRTVAAPSVFIFPPSDEQLKSGTASVVCLLNNF |
| | | YPREAKVQWKVDNALQSGNSQESVTEQDSKDSTY |
| | | SLSSTLTLSKADYEKHKVYACEVTHQGLSSPVTKS |
| | | FNRGEC |
| 200 | Anti-hPD-1-J Heavy Chain | QVQLVQSGAEVKKPGASVKVSCKASGYSFTSYWM |
| | | NWVRQAPGQGLEWIGVIHPSDSETWLDQKFKDRV |
| | | TITVDKSTSTAYMELSSLRSEDTAVYYCAREHYGT |
| | | SPFAYWGQGTLVTVSSASTKGPSVFPLAPCSRSTSE |
| | | STAALGCLVKDYFPEPVTVSWNSGALTSGVHTFPA |
| | | VLQSSGLYSLSSVVTVPSSSLGTKTYTCNVDHKPSN |
| | | TKVDKRVESKYGPPCPPCPAPEFLGGPSVFLFPPKP |
| | | KDTLMISRTPEVTCVVVDVSQEDPEVQFNWYVDG |
| | | VEVHNAKTKPREEQFNSTYRVVSVLTVLHQDWLN |
| | | GKEYKCKVSNKGLPSSIEKTISKAKGQPREPQVYTL |
| | | PPSQEEMTKNQVSLTCLVKGFYPSDIAVEWESNGQ |
| | | PENNYKTTPPVLDSDGSFFLYSRLTVDKSRWQEGN |
| | | VFSCSVMHEALHNHYTQKSLSLSLG |
| 201 | Anti-hPD-1-A-1 Heavy Chain | QVQLVESGGGVVQPGRSLRLDCKASGITFSNSGMH |
| | | WVRQAPGKGLEWVAVIWYDGSKRYYADSVKGRF |
| | | TISRDNSKNTLFLQMNSLRAEDTAVYYCATNDDY |
| | | WGQGTLVTVSSASTKGPSVFPLAPCSRSTSESTAAL |
| | | GCLVKDYFPEPVTVSWNSGALTSGVHTFPAVLQSS |
| | | GLYSLSSVVTVPSSSLGTKTYTCNVDHKPSNTKVD |
| | | KRVESKYGPPCPPCPAPEFLGGPSVFLFPPKPKDTL |
| | | MISRTPEVTCVVVDVSQEDPEVQFNWYVDGVEVH |
| | | NAKTKPREEQFNSTYRVVSVLTVLHQDWLNGKEY |
| | | KCKVSNKGLPSSIEKTISKAKGQPREPQVYTLPPSQ |
| | | EEMTKNQVSLSCAVKGFYPSDIAVEWESNGQPENN |
| | | YKTTPPVLDSDGSFFLVSRLTVDKSRWQEGNVFSC |
| | | SVMHEALHNHYTQKSLSLSLGK |
| 202 | Anti-hPD-1-hIL-2 (SEQ ID | QGQLVQSGAEVKKPGASVKVSCKASGYTFTDYEM |
| | NO: 80) Heavy Chain A18 | HWVRQAPIHGLEWIGVIESETGGTAYNQKFKGRVT |
| | | ITADKSTSTAYMELSSLRSEDTAVYYCAREGITTVA |
| | | TTYYWYFDVWGQGTTVTVSSASTKGPSVFPLAPCS |
| | | RSTSESTAALGCLVKDYFPEPVTVSWNSGALTSGV |
| | | HTFPAVLQSSGLYSLSSVVTVPSSSLGTKTYTCNVD |
| | | HKPSNTKVDKRVESKYGPPCPPCPAPEFLGGPSVFL |
| | | FPPKPKDTLMISRTPEVTCVVVDVSQEDPEVQFNW |
| | | YVDGVEVHNAKTKPREEQFNSTYRVVSVLTVLHQ |
| | | DWLNGKEYKCKVSNKGLPSSIEKTISKAKGQPREP |
| | | QVCTLPPSQEEMTKNQVSLSCAVKGFYPSDIAVEW |
| | | ESNGQPENNYKTTPPVLDSDGSFFLVSRLTVDKSR |
| | | WQEGNVFSCSVMHEALHNHYTQKSLSLSLGAGGG |
| | | GSGGGGSGGGGSAPTSSSTKKTQLQLKHLLLDLQM |
| | | ILNGIQSMEIDATTFKFYMPKKATELKHLQCLEEEL |
| | | KPLEEVLNLAQSKNFHLRPRDLISNINVAVLELKGS |
| | | ETTFMCEYADETATIVEFLNRWITFSQSIISTLT |
| 203 | Anti-hPD-1-hIL-2 (SEQ ID | QVQLVESGGGVVQPGRSLRLDCKASGITFSNSGMH |
| | NO: 70) Heavy Chain A19 | WVRQAPGKGLEWVAVIWYDGSKRYYADSVKGRF |
| | | TISRDNSKNTLFLQMNSLRAEDTAVYYCATNDDY |
| | | WGQGTLVTVSSASTKGPSVFPLAPCSRSTSESTAAL |
| | | GCLVKDYFPEPVTVSWNSGALTSGVHTFPAVLQSS |
| | | GLYSLSSVVTVPSSSLGTKTYTCNVDHKPSNTKVD |
| | | KRVESKYGPPCPPCPAPEFLGGPSVFLFPPKPKDTL |
| | | MISRTPEVTCVVVDVSQEDPEVQFNWYVDGVEVH |
| | | NAKTKPREEQFNSTYRVVSVLTVLHQDWLNGKEY |
| | | KCKVSNKGLPSSIEKTISKAKGQPREPQVYTLPPSQ |
| | | EEMTKNQVSLWCLVKGFYPSDIAVEWESNGQPEN |
| | | NYKTTPPVLDSDGSFFLYSRLTVDKSRWQEGNVFS |
| | | CSVMHEALHNHYTQKSLSLSLGKGGGGSGGGGSG |
| | | GGGSAPTSSSTKKTQLQLEHLLLDLQMILNGIQSME |
| | | IDATTFKFYMPKKATELKHLQCLEEELKPLEEVLNL |
| | | AQSKNFHLRPRDLISNINVAVLELKGSETTFMCEYA |
| | | DETATIVEFLNRWITFSQSIISTLT |
| 204 | Anti-hPD-1-hIL-2 (SEQ ID | QVQLVESGGGVVQPGRSLRLDCKASGITFSNSGMH |
| | NO: 256) Heavy Chain A20 | WVRQAPGKGLEWVAVIWYDGSKRYYADSVKGRF |
| | | TISRDNSKNTLFLQMNSLRAEDTAVYYCATNDDY |
| | | WGQGTLVTVSSASTKGPSVFPLAPCSRSTSESTAAL |
| | | GCLVKDYFPEPVTVSWNSGALTSGVHTFPAVLQSS |
| | | GLYSLSSVVTVPSSSLGTKTYTCNVDHKPSNTKVD |
| | | KRVESKYGPPCPPCPAPEFLGGPSVFLFPPKPKDTL |
| | | MISRTPEVTCVVVDVSQEDPEVQFNWYVDGVEVH |
| | | NAKTKPREEQFNSTYRVVSVLTVLHQDWLNGKEY |
| | | KCKVSNKGLPSSIEKTISKAKGQPREPQVYTLPPSQ |
| | | EEMTKNQVSLWCLVKGFYPSDIAVEWESNGQPEN |
| | | NYKTTPPVLDSDGSFFLYSRLTVDKSRWQEGNVFS |
| | | CSVMHEALHNHYTQKSLSLSLGKGGGGSGGGGSG |
| | | GGGSAPTSSSTKKTQLQLKHLLLDLQMILNGIQSM |
| | | EIDATTFKFYMPKKATELKHLQCLEEELKPLEEVLN |
| | | LAQSKNFHLRPRDLISAINVIVLELKGSETTFMCEY |
| | | ADETATIVEFLNRWITFSQSIISTLT |
| 205 | Anti-hPD-1-hIL-2 (SEQ ID | QVQLVESGGGVVQPGRSLRLDCKASGITFSNSGMH |
| | NO: 256) Heavy Chain A21 | WVRQAPGKGLEWVAVIWYDGSKRYYADSVKGRF |
| | | TISRDNSKNTLFLQMNSLRAEDTAVYYCATNDDY |
| | | WGQGTLVTVSSASTKGPSVFPLAPCSRSTSESTAAL |
| | | GCLVKDYFPEPVTVSWNSGALTSGVHTFPAVLQSS |
| | | GLYSLSSVVTVPSSSLGTKTYTCNVDHKPSNTKVD |
| | | KRVESKYGPPCPPCPAPEFLGGPSVFLFPPKPKDTL |
| | | MISRTPEVTCVVVDVSQEDPEVQFNWYVDGVEVH |
| | | NAKTKPREEQFNSTYRVVSVLTVLHQDWLNGKEY |
| | | KCKVSNKGLPSSIEKTISKAKGQPREPQVYTLPPSQ |
| | | EEMTKNQVSLTCLVKGFYPSDIAVEWESNGQPENN |
| | | YKTTPPVLDSDGSFFLYSRLTVDKSRWQEGNVFSC |
| | | SVMHEALHNHYTQKSLSLSLGKGGGGSGGGGSGG |
| | | GGSAPTSSSTKKTQLQLKHLLLDLQMILNGIQSMEI |
| | | DATTFKFYMPKKATELKHLQCLEEELKPLEEVLNL |
| | | AQSKNFHLRPRDLISAINVIVLELKGSETTFMCEYA |
| | | DETATIVEFLNRWITFSQSIISTLT |
| | GSG Linker | GSG |
| 206 | (GSG)₂ Linker | GSGGSG |
| 207 | (GSG)₃ Linker | GSGGSGGSG |
| 208 | (GSG)₄ Linker | GSGGSGGSGGSG |
| 209 | G3S Linker | GGGS |
| 210 | (G3S)₂ Linker | GGGSGGGS |
| 211 | (G3S)₃ Linker | GGGSGGGSGGGS |
| 212 | (G3S)₄ Linker | GGGSGGGSGGGSGGGS |
| 213 | G4S Linker | GGGGS |
| 214 | (G4S)₂ Linker | GGGGSGGGGS |
| 215 | (G4S)₃ Linker | GGGGSGGGGSGGGGS |
| 216 | (G4S)₄ Linker | GGGGSGGGGSGGGGSGGGGS |
| 217 | SGSG Linker | SGSG |
| 218 | S(GSG)₂ Linker | SGSGGSG |
| 219 | S(GSG)₃ Linker | SGSGGSGGSG |
| 220 | S(GSG)₄ Linker | SGSGGSGGSGGSG |
| 221 | SG3S Linker | SGGGS |
| 222 | S(G3S)₂ Linker | SGGGSGGGS |
| 223 | S(G3S)₃ Linker | SGGGSGGGSGGGS |
| 224 | S(G3S)₄ Linker | SGGGSGGGSGGGSGGGS |
| 225 | SG4S Linker | SGGGGS |
| 226 | S(G4S)₂ Linker | SGGGGSGGGGS |
| 227 | S(G4S)₃ Linker | SGGGGSGGGGSGGGGS |
| 228 | S(G4S)₄ Linker | SGGGGSGGGGSGGGGSGGGGS |
| 229 | EAAAK Linker | EAAAK |
| 230 | (EAAAK)₂ Linker | EAAAKEAAAK |
| 231 | (EAAAK)₃ Linker | EAAAKEAAAKEAAAK |
| 232 | (EAAAK)₄ Linker | EAAAKEAAAKEAAAKEAAAK |
| 233 | PAPAP Linker | PAPAP |
| 234 | (PAPAP)₂ Linker | PAPAPPAPAP |
| 235 | (PAPAP)₃ Linker | PAPAPPAPAPPAPAP |
| 236 | (PAPAP)₄ Linker | PAPAPPAPAPPAPAPPAPAP |
| 237 | VLVH Linker | IKRTVAAP |
| 238 | RAKPS Linker | RAKPS |
| 239 | (RAKPS)₂ Linker | RAKPSRAKPS |
| 240 | ASTKG Linker | ASTKG |
| 241 | (ASTKG)₂ Linker | ASTKGASTKG |
| 242 | AKTHT Linker | AKTHT |
| 243 | (AKTHT)₂ Linker | AKTHTAKTHT |
| 244 | ASTKG(G4S)₂ Linker | ASTKGGGGGSGGGGS |
| 245 | RAKPS(G4S)₂ Linker | RAKPSGGGGSGGGGS |
| 246 | hIL-2-anti-HSA C1 | APTSSSTKKTQLQLKHLLLDLQMILNGIQSMHIDAT |
| | | TFKFYMPKKATELKHLQCLEEELKPLEEVLNLAQS |
| | | KNFHLRPRDLIDNINVIVLELKGSETTFMCEYADET |
| | | ATIVEFLNRWITFSQSIISTLTGSGGSGGSGGSGEVQ |
| | | LVESGGGLVQPGGSLRLSCAASGSTWSINTLAWYR |
| | | QAPGKQRDLVARISSGGSTYYADSVKGRFTISRDN |
| | | SKNTLYLQMNSLRAEDTAVYYCYAQSTWYPPSWG |
| | | QGTLVTVSS |
| 247 | Anti-hPD-1 Heavy Chain | QGQLVQSGAEVKKPGASVKVSCKASGYTFTDYEM |
| | (IgG1, N297A) | HWVRQAPIHGLEWIGVIESETGGTAYNQKFKGRVT |
| | | ITADKSTSTAYMELSSLRSEDTAVYYCAREGITTVA |
| | | TTYYWYFDVWGQGTTVTVSSASTKGPSVFPLAPSS |
| | | KSTSGGTAALGCLVKDYFPEPVTVSWNSGALTSGV |
| | | HTFPAVLQSSGLYSLSSVVTVPSSSLGTQTYICNVN |
| | | HKPSNTKVDKKVEPKSCDKTHTCPPCPAPELLGGP |
| | | SVFLFPPKPKDTLMISRTPEVTCVVVDVSHEDPEVK |
| | | FNWYVDGVEVHNAKTKPREEQYASTYRVVSVLTV |
| | | LHQDWLNGKEYKCKVSNKALPAPIEKTISKAKGQP |
| | | REPQVYTLPPSREEMTKNQVSLTCLVKGFYPSDIAV |
| | | EWESNGQPENNYKTTPPVLDSDGSFFLYSKLTVDK |
| | | SRWQQGNVFSCSVMHEALHNHYTQKSLSLSPGK |
| 248 | Anti-hPD-1 Heavy Chain | QGQLVQSGAEVKKPGASVKVSCKASGYTFTDYEM |
| | (IgG1, N297A, Last K to A) | HWVRQAPIHGLEWIGVIESETGGTAYNQKFKGRVT |
| | | ITADKSTSTAYMELSSLRSEDTAVYYCAREGITTVA |
| | | TTYYWYFDVWGQGTTVTVSSASTKGPSVFPLAPSS |
| | | KSTSGGTAALGCLVKDYFPEPVTVSWNSGALTSGV |
| | | HTFPAVLQSSGLYSLSSVVTVPSSSLGTQTYICNVN |
| | | HKPSNTKVDKKVEPKSCDKTHTCPPCPAPELLGGP |
| | | SVFLFPPKPKDTLMISRTPEVTCVVVDVSHEDPEVK |
| | | FNWYVDGVEVHNAKTKPREEQYASTYRVVSVLTV |
| | | LHQDWLNGKEYKCKVSNKALPAPIEKTISKAKGQP |
| | | REPQVYTLPPCREEMTKNQVSLWCLVKGFYPSDIA |
| | | VEWESNGQPENNYKTTPPVLDSDGSFFLYSKLTVD |
| | | KSRWQQGNVFSCSVMHEALHNHYTQKSLSLSPGA |
| 249 | Anti-hPD-1-hIL-2 Heavy | QGQLVQSGAEVKKPGASVKVSCKASGYTFTDYEM |
| | Chain A1 | HWVRQAPIHGLEWIGVIESETGGTAYNQKFKGRVT |
| | (IgG1, N297A, Last K to A) | ITADKSTSTAYMELSSLRSEDTAVYYCAREGITTVA |
| | | TTYYWYFDVWGQGTTVTVSSASTKGPSVFPLAPSS |
| | | KSTSGGTAALGCLVKDYFPEPVTVSWNSGALTSGV |
| | | HTFPAVLQSSGLYSLSSVVTVPSSSLGTQTYICNVN |
| | | HKPSNTKVDKKVEPKSCDKTHTCPPCPAPELLGGP |
| | | SVFLFPPKPKDTLMISRTPEVTCVVVDVSHEDPEVK |
| | | FNWYVDGVEVHNAKTKPREEQYASTYRVVSVLTV |
| | | LHQDWLNGKEYKCKVSNKALPAPIEKTISKAKGQP |
| | | REPQVCTLPPSREEMTKNQVSLSCAVKGFYPSDIAV |
| | | EWESNGQPENNYKTTPPVLDSDGSFFLVSKLTVDK |
| | | SRWQQGNVFSCSVMHEALHNHYTQKSLSLSPGAG |
| | | GGGSGGGGSGGGGSAPTSSSTKKTQLQLEHLLLDL |
| | | QMILNGIQSMEIDATTFKFYMPKKATELKHLQCLE |
| | | EELKPLEEVLNLAQSKNFHLRPRDLISNINVIVLELK |
| | | GSETTFMCEYADETATIVEFLNRWITFSQSIISTLT |
| 250 | Anti-hPD-1-hIL-2 Heavy | QGQLVQSGAEVKKPGASVKVSCKASGYTFTDYEM |
| | Chain A2 | HWVRQAPIHGLEWIGVIESETGGTAYNQKFKGRVT |
| | (IgG1, N297A, Last K to A) | ITADKSTSTAYMELSSLRSEDTAVYYCAREGITTVA |
| | | TTYYWYFDVWGQGTTVTVSSASTKGPSVFPLAPSS |
| | | KSTSGGTAALGCLVKDYFPEPVTVSWNSGALTSGV |
| | | HTFPAVLQSSGLYSLSSVVTVPSSSLGTQTYICNVN |
| | | HKPSNTKVDKKVEPKSCDKTHTCPPCPAPELLGGP |
| | | SVFLFPPKPKDTLMISRTPEVTCVVVDVSHEDPEVK |
| | | FNWYVDGVEVHNAKTKPREEQYASTYRVVSVLTV |
| | | LHQDWLNGKEYKCKVSNKALPAPIEKTISKAKGQP |
| | | REPQVCTLPPSREEMTKNQVSLSCAVKGFYPSDIAV |
| | | EWESNGQPENNYKTTPPVLDSDGSFFLVSKLTVDK |
| | | SRWQQGNVFSCSVMHEALHNHYTQKSLSLSPGAG |
| | | GGGSGGGGSGGGGSAPTSSSTKKTQLQLEELLLDL |
| | | QMILNGIQSMEIDATTFKFYMPKKATELKHLQCLE |
| | | EELKPLEEVLNLAQSKNFHLRPRDLISNINVIVLELK |
| | | GSETTFMCEYADETATIVEFLNRWITFSQSIISTLT |
| 251 | Anti-hPD-1-hIL-2 (SEQ ID | QGQLVQSGAEVKKPGASVKVSCKASGYTFTDYEM |
| | NO: 68) Heavy Chain A3 | HWVRQAPIHGLEWIGVIESETGGTAYNQKFKGRVT |
| | (IgG1, N297A, Last K to A) | ITADKSTSTAYMELSSLRSEDTAVYYCAREGITTVA |
| | | TTYYWYFDVWGQGTTVTVSSASTKGPSVFPLAPSS |
| | | KSTSGGTAALGCLVKDYFPEPVTVSWNSGALTSGV |
| | | HTFPAVLQSSGLYSLSSVVTVPSSSLGTQTYICNVN |
| | | HKPSNTKVDKKVEPKSCDKTHTCPPCPAPELLGGP |
| | | SVFLFPPKPKDTLMISRTPEVTCVVVDVSHEDPEVK |
| | | FNWYVDGVEVHNAKTKPREEQYASTYRVVSVLTV |
| | | LHQDWLNGKEYKCKVSNKALPAPIEKTISKAKGQP |
| | | REPQVCTLPPSREEMTKNQVSLSCAVKGFYPSDIAV |
| | | EWESNGQPENNYKTTPPVLDSDGSFFLVSKLTVDK |
| | | SRWQQGNVFSCSVMHEALHNHYTQKSLSLSPGAG |
| | | GGGSGGGGSGGGGSAPTSSSTKKTQLQLEHLLLDL |
| | | QMILNGIQSMEIDATTFKFYMPKKATELKHLQCLE |
| | | EELKPLEEVLNLAQSKNFHLRPRDLISAINVIVLELK |
| | | GSETTFMCEYADETATIVEFLNRWITFSQSIISTLT |
| 252 | Anti-hPD-1-hIL-2 (SEQ ID | QGQLVQSGAEVKKPGASVKVSCKASGYTFTDYEM |
| | NO: 80) Heavy Chain A4 | HWVRQAPIHGLEWIGVIESETGGTAYNQKFKGRVT |
| | (IgG1, N297A, Last K to A) | ITADKSTSTAYMELSSLRSEDTAVYYCAREGITTVA |
| | | TTYYWYFDVWGQGTTVTVSSASTKGPSVFPLAPSS |
| | | KSTSGGTAALGCLVKDYFPEPVTVSWNSGALTSGV |
| | | HTFPAVLQSSGLYSLSSVVTVPSSSLGTQTYICNVN |
| | | HKPSNTKVDKKVEPKSCDKTHTCPPCPAPELLGGP |
| | | SVFLFPPKPKDTLMISRTPEVTCVVVDVSHEDPEVK |
| | | FNWYVDGVEVHNAKTKPREEQYASTYRVVSVLTV |
| | | LHQDWLNGKEYKCKVSNKALPAPIEKTISKAKGQP |
| | | REPQVCTLPPSREEMTKNQVSLSCAVKGFYPSDIAV |
| | | EWESNGQPENNYKTTPPVLDSDGSFFLVSKLTVDK |
| | | SRWQQGNVFSCSVMHEALHNHYTQKSLSLSPGAG |
| | | GGGSGGGGSGGGGSAPTSSSTKKTQLQLKHLLLDL |
| | | QMILNGIQSMEIDATTFKFYMPKKATELKHLQCLE |
| | | EELKPLEEVLNLAQSKNFHLRPRDLISNINVAVLEL |
| | | KGSETTFMCEYADETATIVEFLNRWITFSQSIISTLT |
| 253 | Anti-hPD-1-hIL-2 (SEQ ID | QGQLVQSGAEVKKPGASVKVSCKASGYTFTDYEM |
| | NO: 70) Heavy Chain A5 | HWVRQAPIHGLEWIGVIESETGGTAYNQKFKGRVT |
| | (IgG1, N297A, Last K to A) | ITADKSTSTAYMELSSLRSEDTAVYYCAREGITTVA |
| | | TTYYWYFDVWGQGTTVTVSSASTKGPSVFPLAPSS |
| | | KSTSGGTAALGCLVKDYFPEPVTVSWNSGALTSGV |
| | | HTFPAVLQSSGLYSLSSVVTVPSSSLGTQTYICNVN |
| | | HKPSNTKVDKKVEPKSCDKTHTCPPCPAPELLGGP |
| | | SVFLFPPKPKDTLMISRTPEVTCVVVDVSHEDPEVK |
| | | FNWYVDGVEVHNAKTKPREEQYASTYRVVSVLTV |
| | | LHQDWLNGKEYKCKVSNKALPAPIEKTISKAKGQP |
| | | REPQVCTLPPSREEMTKNQVSLSCAVKGFYPSDIAV |
| | | EWESNGQPENNYKTTPPVLDSDGSFFLVSKLTVDK |
| | | SRWQQGNVFSCSVMHEALHNHYTQKSLSLSPGAG |
| | | GGGSGGGGSGGGGSAPTSSSTKKTQLQLEHLLLDL |
| | | QMILNGIQSMEIDATTFKFYMPKKATELKHLQCLE |
| | | EELKPLEEVLNLAQSKNFHLRPRDLISNINVAVLEL |
| | | KGSETTFMCEYADETATIVEFLNRWITFSQSIISTLT |
| 254 | Anti-hPD-1-hIL-2 Heavy | QGQLVQSGAEVKKPGASVKVSCKASGYTFTDYEM |
| | Chain A6 | HWVRQAPIHGLEWIGVIESETGGTAYNQKFKGRVT |
| | (IgG1, N297A, Last K to A) | ITADKSTSTAYMELSSLRSEDTAVYYCAREGITTVA |
| | | TTYYWYFDVWGQGTTVTVSSASTKGPSVFPLAPSS |
| | | KSTSGGTAALGCLVKDYFPEPVTVSWNSGALTSGV |
| | | HTFPAVLQSSGLYSLSSVVTVPSSSLGTQTYICNVN |
| | | HKPSNTKVDKKVEPKSCDKTHTCPPCPAPELLGGP |
| | | SVFLFPPKPKDTLMISRTPEVTCVVVDVSHEDPEVK |
| | | FNWYVDGVEVHNAKTKPREEQYASTYRVVSVLTV |
| | | LHQDWLNGKEYKCKVSNKALPAPIEKTISKAKGQP |
| | | REPQVCTLPPSREEMTKNQVSLSCAVKGFYPSDIAV |
| | | EWESNGQPENNYKTTPPVLDSDGSFFLVSKLTVDK |
| | | SRWQQGNVFSCSVMHEALHNHYTQKSLSLSPGAG |
| | | GGGSGGGGSGGGGSAPTSSSTKKTQLQLKHLLLDL |
| | | QMILNGIQSMHIDATTFKFYMPKKATELKHLQCLE |
| | | EELKPLEEVLNLAQSKNFHLRPRDLIDNINVIVLEL |
| | | KGSETTFMCEYADETATIVEFLNRWITFSQSIISTLT |
| 255 | IL-2-IL-15 Chimera 41 | APTSSSTKKTQLQLKILLLDLQMILNGIQSMEIDATT |
| | (E15K, H16I, C125S) | FKFYMPKKATELKHLQCLEEELKPLEEVLNLAQSK |
| | | NFHLRPRDLISNINVIVLELKGSETTFMCEYADETA |
| | | TIVEFLNRWITFSQSIISTLT |
| 256 | IL-2-IL-15 Chimera 42 | APTSSSTKKTQLQLKHLLLDLQMILNGIQSMEIDAT |
| | (E15K, N88A, C125S) | TFKFYMPKKATELKHLQCLEEELKPLEEVLNLAQS |
| | | KNFHLRPRDLISAINVIVLELKGSETTFMCEYADET |
| | | ATIVEFLNRWITFSQSIISTLT |
| 257 | Anti-hPD-1 Heavy Chain 1 | QGQLVQSGAEVKKPGASVKVSCKASGYTFTDYEM |
| | (hlgG4) | HWVRQAPIHGLEWIGVIESETGGTAYNQKFKGRVT |
| | | ITADKSTSTAYMELSSLRSEDTAVYYCAREGITTVA |
| | | TTYYWYFDVWGQGTTVTVSSASTKGPSVFPLAPCS |
| | | RSTSESTAALGCLVKDYFPEPVTVSWNSGALTSGV |
| | | HTFPAVLQSSGLYSLSSVVTVPSSSLGTKTYTCNVD |
| | | HKPSNTKVDKRVESKYGPPCPPCPAPEFLGGPSVFL |
| | | FPPKPKDTLMISRTPEVTCVVVDVSQEDPEVQFNW |
| | | YVDGVEVHNAKTKPREEQFNSTYRVVSVLTVLHQ |
| | | DWLNGKEYKCKVSNKGLPSSIEKTISKAKGQPREP |
| | | QVYTLPPSQEEMTKNQVSLWCLVKGFYPSDIAVE |
| | | WESNGQPENNYKTTPPVLDSDGSFFLYSRLTVDKS |
| | | RWQEGNVFSCSVMHEALHNHYTQKSLSLSLGA |
| 258 | Anti-hPD-1-hIL-2 (SEQ ID | QGQLVQSGAEVKKPGASVKVSCKASGYTFTDYEM |
| | NO: 56) Heavy Chain A7 | HWVRQAPIHGLEWIGVIESETGGTAYNQKFKGRVT |
| | | ITADKSTSTAYMELSSLRSEDTAVYYCAREGITTVA |
| | | TTYYWYFDVWGQGTTVTVSSASTKGPSVFPLAPCS |
| | | RSTSESTAALGCLVKDYFPEPVTVSWNSGALTSGV |
| | | HTFPAVLQSSGLYSLSSVVTVPSSSLGTKTYTCNVD |
| | | HKPSNTKVDKRVESKYGPPCPPCPAPEFLGGPSVFL |
| | | FPPKPKDTLMISRTPEVTCVVVDVSQEDPEVQFNW |
| | | YVDGVEVHNAKTKPREEQFNSTYRVVSVLTVLHQ |
| | | DWLNGKEYKCKVSNKGLPSSIEKTISKAKGQPREP |
| | | QVYTLPPSQEEMTKNQVSLSCAVKGFYPSDIAVEW |
| | | ESNGQPENNYKTTPPVLDSDGSFFLVSRLTVDKSR |
| | | WQEGNVFSCSVMHEALHNHYTQKSLSLSLGAGGG |
| | | GSGGGGSGGGGSAPTSSSTKKTQLQLEILLLDLQMI |
| | | LNGIQSMEIDATTFKFYMPKKATELKHLQCLEEEL |
| | | KPLEEVLNLAQSKNFHLRPRDLISNINVIVLELKGSE |
| | | TTFMCEYADETATIVEFLNRWITFSQSIISTLT |
| 259 | Anti-hPD-1-hIL-2 (SEQ ID | QGQLVQSGAEVKKPGASVKVSCKASGYTFTDYEM |
| | NO: 255) Heavy Chain A8 | HWVRQAPIHGLEWIGVIESETGGTAYNQKFKGRVT |
| | | ITADKSTSTAYMELSSLRSEDTAVYYCAREGITTVA |
| | | TTYYWYFDVWGQGTTVTVSSASTKGPSVFPLAPCS |
| | | RSTSESTAALGCLVKDYFPEPVTVSWNSGALTSGV |
| | | HTFPAVLQSSGLYSLSSVVTVPSSSLGTKTYTCNVD |
| | | HKPSNTKVDKRVESKYGPPCPPCPAPEFLGGPSVFL |
| | | FPPKPKDTLMISRTPEVTCVVVDVSQEDPEVQFNW |
| | | YVDGVEVHNAKTKPREEQFNSTYRVVSVLTVLHQ |
| | | DWLNGKEYKCKVSNKGLPSSIEKTISKAKGQPREP |
| | | QVYTLPPSQEEMTKNQVSLSCAVKGFYPSDIAVEW |
| | | ESNGQPENNYKTTPPVLDSDGSFFLVSRLTVDKSR |
| | | WQEGNVFSCSVMHEALHNHYTQKSLSLSLGAGGG |
| | | GSGGGGSGGGGSAPTSSSTKKTQLQLKILLLDLQMI |
| | | LNGIQSMEIDATTFKFYMPKKATELKHLQCLEEEL |
| | | KPLEEVLNLAQSKNFHLRPRDLISNINVIVLELKGSE |
| | | TTFMCEYADETATIVEFLNRWITFSQSIISTLT |
| 260 | Anti-hPD-1-hIL-2 (SEQ ID | QGQLVQSGAEVKKPGASVKVSCKASGYTFTDYEM |
| | NO: 68) Heavy Chain A9 | HWVRQAPIHGLEWIGVIESETGGTAYNQKFKGRVT |
| | | ITADKSTSTAYMELSSLRSEDTAVYYCAREGITTVA |
| | | TTYYWYFDVWGQGTTVTVSSASTKGPSVFPLAPCS |
| | | RSTSESTAALGCLVKDYFPEPVTVSWNSGALTSGV |
| | | HTFPAVLQSSGLYSLSSVVTVPSSSLGTKTYTCNVD |
| | | HKPSNTKVDKRVESKYGPPCPPCPAPEFLGGPSVFL |
| | | FPPKPKDTLMISRTPEVTCVVVDVSQEDPEVQFNW |
| | | YVDGVEVHNAKTKPREEQFNSTYRVVSVLTVLHQ |
| | | DWLNGKEYKCKVSNKGLPSSIEKTISKAKGQPREP |
| | | QVYTLPPSQEEMTKNQVSLSCAVKGFYPSDIAVEW |
| | | ESNGQPENNYKTTPPVLDSDGSFFLVSRLTVDKSR |
| | | WQEGNVFSCSVMHEALHNHYTQKSLSLSLGAGGG |
| | | GSGGGGSGGGGSAPTSSSTKKTQLQLEHLLLDLQM |
| | | ILNGIQSMEIDATTFKFYMPKKATELKHLQCLEEEL |
| | | KPLEEVLNLAQSKNFHLRPRDLISAINVIVLELKGSE |
| | | TTFMCEYADETATIVEFLNRWITFSQSIISTLT |
| 261 | Anti-hPD-1-hIL-2 (SEQ ID | QGQLVQSGAEVKKPGASVKVSCKASGYTFTDYEM |
| | NO: 256) Heavy Chain A10 | HWVRQAPIHGLEWIGVIESETGGTAYNQKFKGRVT |
| | | ITADKSTSTAYMELSSLRSEDTAVYYCAREGITTVA |
| | | TTYYWYFDVWGQGTTVTVSSASTKGPSVFPLAPCS |
| | | RSTSESTAALGCLVKDYFPEPVTVSWNSGALTSGV |
| | | HTFPAVLQSSGLYSLSSVVTVPSSSLGTKTYTCNVD |
| | | HKPSNTKVDKRVESKYGPPCPPCPAPEFLGGPSVFL |
| | | FPPKPKDTLMISRTPEVTCVVVDVSQEDPEVQFNW |
| | | YVDGVEVHNAKTKPREEQFNSTYRVVSVLTVLHQ |
| | | DWLNGKEYKCKVSNKGLPSSIEKTISKAKGQPREP |
| | | QVYTLPPSQEEMTKNQVSLSCAVKGFYPSDIAVEW |
| | | ESNGQPENNYKTTPPVLDSDGSFFLVSRLTVDKSR |
| | | WQEGNVFSCSVMHEALHNHYTQKSLSLSLGAGGG |
| | | GSGGGGSGGGGSAPTSSSTKKTQLQLKHLLLDLQM |
| | | ILNGIQSMEIDATTFKFYMPKKATELKHLQCLEEEL |
| | | KPLEEVLNLAQSKNFHLRPRDLISAINVIVLELKGSE |
| | | TTFMCEYADETATIVEFLNRWITFSQSIISTLT |
| 262 | Anti-hPD-1-hIL-2 (SEQ ID | QGQLVQSGAEVKKPGASVKVSCKASGYTFTDYEM |
| | NO: 70) Heavy Chain A11 | HWVRQAPIHGLEWIGVIESETGGTAYNQKFKGRVT |
| | | ITADKSTSTAYMELSSLRSEDTAVYYCAREGITTVA |
| | | TTYYWYFDVWGQGTTVTVSSASTKGPSVFPLAPCS |
| | | RSTSESTAALGCLVKDYFPEPVTVSWNSGALTSGV |
| | | HTFPAVLQSSGLYSLSSVVTVPSSSLGTKTYTCNVD |
| | | HKPSNTKVDKRVESKYGPPCPPCPAPEFLGGPSVFL |
| | | FPPKPKDTLMISRTPEVTCVVVDVSQEDPEVQFNW |
| | | YVDGVEVHNAKTKPREEQFNSTYRVVSVLTVLHQ |
| | | DWLNGKEYKCKVSNKGLPSSIEKTISKAKGQPREP |
| | | QVYTLPPSQEEMTKNQVSLSCAVKGFYPSDIAVEW |
| | | ESNGQPENNYKTTPPVLDSDGSFFLVSRLTVDKSR |
| | | WQEGNVFSCSVMHEALHNHYTQKSLSLSLGAGGG |
| | | GSGGGGSGGGGSAPTSSSTKKTQLQLEHLLLDLQM |
| | | ILNGIQSMEIDATTFKFYMPKKATELKHLQCLEEEL |
| | | KPLEEVLNLAQSKNFHLRPRDLISNINVAVLELKGS |
| | | ETTFMCEYADETATIVEFLNRWITFSQSIISTLT |
| 263 | Anti-hPD-1-hIL-2 (SEQ ID | QGQLVQSGAEVKKPGASVKVSCKASGYTFTDYEM |
| | NO: 80) Heavy Chain A12 | HWVRQAPIHGLEWIGVIESETGGTAYNQKFKGRVT |
| | | ITADKSTSTAYMELSSLRSEDTAVYYCAREGITTVA |
| | | TTYYWYFDVWGQGTTVTVSSASTKGPSVFPLAPCS |
| | | RSTSESTAALGCLVKDYFPEPVTVSWNSGALTSGV |
| | | HTFPAVLQSSGLYSLSSVVTVPSSSLGTKTYTCNVD |
| | | HKPSNTKVDKRVESKYGPPCPPCPAPEFLGGPSVFL |
| | | FPPKPKDTLMISRTPEVTCVVVDVSQEDPEVQFNW |
| | | YVDGVEVHNAKTKPREEQFNSTYRVVSVLTVLHQ |
| | | DWLNGKEYKCKVSNKGLPSSIEKTISKAKGQPREP |
| | | QVYTLPPSQEEMTKNQVSLSCAVKGFYPSDIAVEW |
| | | ESNGQPENNYKTTPPVLDSDGSFFLVSRLTVDKSR |
| | | WQEGNVFSCSVMHEALHNHYTQKSLSLSLGAGGG |
| | | GSGGGGSGGGGSAPTSSSTKKTQLQLKHLLLDLQM |
| | | ILNGIQSMEIDATTFKFYMPKKATELKHLQCLEEEL |
| | | KPLEEVLNLAQSKNFHLRPRDLISNINVAVLELKGS |
| | | ETTFMCEYADETATIVEFLNRWITFSQSIISTLT |
| 264 | Anti-hPD-1 Heavy Chain 2 | QGQLVQSGAEVKKPGASVKVSCKASGYTFTDYEM |
| | (hIgG4) | HWVRQAPIHGLEWIGVIESETGGTAYNQKFKGRVT |
| | | ITADKSTSTAYMELSSLRSEDTAVYYCAREGITTVA |
| | | TTYYWYFDVWGQGTTVTVSSASTKGPSVFPLAPCS |
| | | RSTSESTAALGCLVKDYFPEPVTVSWNSGALTSGV |
| | | HTFPAVLQSSGLYSLSSVVTVPSSSLGTKTYTCNVD |
| | | HKPSNTKVDKRVESKYGPPCPPCPAPEFLGGPSVFL |
| | | FPPKPKDTLMISRTPEVTCVVVDVSQEDPEVQFNW |
| | | YVDGVEVHNAKTKPREEQFNSTYRVVSVLTVLHQ |
| | | DWLNGKEYKCKVSNKGLPSSIEKTISKAKGQPREP |
| | | QVYTLPPCQEEMTKNQVSLWCLVKGFYPSDIAVE |
| | | WESNGQPENNYKTTPPVLDSDGSFFLYSRLTVDKS |
| | | RWQEGNVFSCSVMHEALHNHYTQKSLSLSLGA |
| 265 | Anti-hPD-1-hIL-2 (SEQ ID | QGQLVQSGAEVKKPGASVKVSCKASGYTFTDYEM |
| | NO: 56) Heavy Chain A13 | HWVRQAPIHGLEWIGVIESETGGTAYNQKFKGRVT |
| | | ITADKSTSTAYMELSSLRSEDTAVYYCAREGITTVA |
| | | TTYYWYFDVWGQGTTVTVSSASTKGPSVFPLAPCS |
| | | RSTSESTAALGCLVKDYFPEPVTVSWNSGALTSGV |
| | | HTFPAVLQSSGLYSLSSVVTVPSSSLGTKTYTCNVD |
| | | HKPSNTKVDKRVESKYGPPCPPCPAPEFLGGPSVFL |
| | | FPPKPKDTLMISRTPEVTCVVVDVSQEDPEVQFNW |
| | | YVDGVEVHNAKTKPREEQFNSTYRVVSVLTVLHQ |
| | | DWLNGKEYKCKVSNKGLPSSIEKTISKAKGQPREP |
| | | QVCTLPPSQEEMTKNQVSLSCAVKGFYPSDIAVEW |
| | | ESNGQPENNYKTTPPVLDSDGSFFLVSRLTVDKSR |
| | | WQEGNVFSCSVMHEALHNHYTQKSLSLSLGAGGG |
| | | GSGGGGSGGGGSAPTSSSTKKTQLQLEILLLDLQMI |
| | | LNGIQSMEIDATTFKFYMPKKATELKHLQCLEEEL |
| | | KPLEEVLNLAQSKNFHLRPRDLISNINVIVLELKGSE |
| | | TTFMCEYADETATIVEFLNRWITFSQSIISTLT |
| 266 | Anti-hPD-1-hIL-2 (SEQ ID | QGQLVQSGAEVKKPGASVKVSCKASGYTFTDYEM |
| | NO: 255) Heavy Chain A14 | HWVRQAPIHGLEWIGVIESETGGTAYNQKFKGRVT |
| | | ITADKSTSTAYMELSSLRSEDTAVYYCAREGITTVA |
| | | TTYYWYFDVWGQGTTVTVSSASTKGPSVFPLAPCS |
| | | RSTSESTAALGCLVKDYFPEPVTVSWNSGALTSGV |
| | | HTFPAVLQSSGLYSLSSVVTVPSSSLGTKTYTCNVD |
| | | HKPSNTKVDKRVESKYGPPCPPCPAPEFLGGPSVFL |
| | | FPPKPKDTLMISRTPEVTCVVVDVSQEDPEVQFNW |
| | | YVDGVEVHNAKTKPREEQFNSTYRVVSVLTVLHQ |
| | | DWLNGKEYKCKVSNKGLPSSIEKTISKAKGQPREP |
| | | QVCTLPPSQEEMTKNQVSLSCAVKGFYPSDIAVEW |
| | | ESNGQPENNYKTTPPVLDSDGSFFLVSRLTVDKSR |
| | | WQEGNVFSCSVMHEALHNHYTQKSLSLSLGAGGG |
| | | GSGGGGSGGGGSAPTSSSTKKTQLQLKILLLDLQMI |
| | | LNGIQSMEIDATTFKFYMPKKATELKHLQCLEEEL |
| | | KPLEEVLNLAQSKNFHLRPRDLISNINVIVLELKGSE |
| | | TTFMCEYADETATIVEFLNRWITFSQSIISTLT |
| 267 | Anti-hPD-1-hIL-2 (SEQ ID | QGQLVQSGAEVKKPGASVKVSCKASGYTFTDYEM |
| | NO: 68) Heavy Chain A15 | HWVRQAPIHGLEWIGVIESETGGTAYNQKFKGRVT |
| | | ITADKSTSTAYMELSSLRSEDTAVYYCAREGITTVA |
| | | TTYYWYFDVWGQGTTVTVSSASTKGPSVFPLAPCS |
| | | RSTSESTAALGCLVKDYFPEPVTVSWNSGALTSGV |
| | | HTFPAVLQSSGLYSLSSVVTVPSSSLGTKTYTCNVD |
| | | HKPSNTKVDKRVESKYGPPCPPCPAPEFLGGPSVFL |
| | | FPPKPKDTLMISRTPEVTCVVVDVSQEDPEVQFNW |
| | | YVDGVEVHNAKTKPREEQFNSTYRVVSVLTVLHQ |
| | | DWLNGKEYKCKVSNKGLPSSIEKTISKAKGQPREP |
| | | QVCTLPPSQEEMTKNQVSLSCAVKGFYPSDIAVEW |
| | | ESNGQPENNYKTTPPVLDSDGSFFLVSRLTVDKSR |
| | | WQEGNVFSCSVMHEALHNHYTQKSLSLSLGAGGG |
| | | GSGGGGSGGGGSAPTSSSTKKTQLQLEHLLLDLQM |
| | | ILNGIQSMEIDATTFKFYMPKKATELKHLQCLEEEL |
| | | KPLEEVLNLAQSKNFHLRPRDLISAINVIVLELKGSE |
| | | TTFMCEYADETATIVEFLNRWITFSQSIISTLT |
| 268 | Anti-hPD-1-hIL-2 (SEQ ID | QGQLVQSGAEVKKPGASVKVSCKASGYTFTDYEM |
| | NO: 256) Heavy Chain A16 | HWVRQAPIHGLEWIGVIESETGGTAYNQKFKGRVT |
| | | ITADKSTSTAYMELSSLRSEDTAVYYCAREGITTVA |
| | | TTYYWYFDVWGQGTTVTVSSASTKGPSVFPLAPCS |
| | | RSTSESTAALGCLVKDYFPEPVTVSWNSGALTSGV |
| | | HTFPAVLQSSGLYSLSSVVTVPSSSLGTKTYTCNVD |
| | | HKPSNTKVDKRVESKYGPPCPPCPAPEFLGGPSVFL |
| | | FPPKPKDTLMISRTPEVTCVVVDVSQEDPEVQFNW |
| | | YVDGVEVHNAKTKPREEQFNSTYRVVSVLTVLHQ |
| | | DWLNGKEYKCKVSNKGLPSSIEKTISKAKGQPREP |
| | | QVCTLPPSQEEMTKNQVSLSCAVKGFYPSDIAVEW |
| | | ESNGQPENNYKTTPPVLDSDGSFFLVSRLTVDKSR |
| | | WQEGNVFSCSVMHEALHNHYTQKSLSLSLGAGGG |
| | | GSGGGGSGGGGSAPTSSSTKKTQLQLKHLLLDLQM |
| | | ILNGIQSMEIDATTFKFYMPKKATELKHLQCLEEEL |
| | | KPLEEVLNLAQSKNFHLRPRDLISAINVIVLELKGSE |
| | | TTFMCEYADETATIVEFLNRWITFSQSIISTLT |
| 269 | Anti-hPD-1-hIL-2 (SEQ ID | QGQLVQSGAEVKKPGASVKVSCKASGYTFTDYEM |
| | NO: 70) Heavy Chain A17 | HWVRQAPIHGLEWIGVIESETGGTAYNQKFKGRVT |
| | | ITADKSTSTAYMELSSLRSEDTAVYYCAREGITTVA |
| | | TTYYWYFDVWGQGTTVTVSSASTKGPSVFPLAPCS |
| | | RSTSESTAALGCLVKDYFPEPVTVSWNSGALTSGV |
| | | HTFPAVLQSSGLYSLSSVVTVPSSSLGTKTYTCNVD |
| | | HKPSNTKVDKRVESKYGPPCPPCPAPEFLGGPSVFL |
| | | FPPKPKDTLMISRTPEVTCVVVDVSQEDPEVQFNW |
| | | YVDGVEVHNAKTKPREEQFNSTYRVVSVLTVLHQ |
| | | DWLNGKEYKCKVSNKGLPSSIEKTISKAKGQPREP |
| | | QVCTLPPSQEEMTKNQVSLSCAVKGFYPSDIAVEW |
| | | ESNGQPENNYKTTPPVLDSDGSFFLVSRLTVDKSR |
| | | WQEGNVFSCSVMHEALHNHYTQKSLSLSLGAGGG |
| | | GSGGGGSGGGGSAPTSSSTKKTQLQLEHLLLDLQM |
| | | ILNGIQSMEIDATTFKFYMPKKATELKHLQCLEEEL |
| | | KPLEEVLNLAQSKNFHLRPRDLISNINVAVLELKGS |
| | | ETTFMCEYADETATIVEFLNRWITFSQSIISTLT |

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

What is claimed is:

1. A fusion protein comprising an interleukin-2 (IL-2) domain, a programmed cell death-1 (PD-1) binding domain, and a half-life-extension domain; wherein the IL-2 domain comprises: (i) a replacement of the amino acid residues from positions N29 to A50 with an IL-15 hinge fragment-containing peptide; (ii) an amino acid substitution at position E15, H16, D20, K32, K76, S87, N88, or I92; (iii) an amino acid substitution at a position from R38 to Y45; and/or (iv) a disulfide bond formed between two amino acid residues from positions N30 to L80.

2. The fusion protein of claim 1, wherein the IL-2 domain comprises a replacement of the amino acid residues from positions N29 to A50 with an IL-15 hinge fragment-containing peptide, and optionally an amino acid substitution at position E15, H16, D20, S87, N88, or I92.

3. The fusion protein of claim 1 or 2, wherein the IL-2 domain comprises a replacement of the amino acid residues from positions N29 to L40 having an amino acid sequence of SEQ ID NO: 39 with an IL-15 hinge fragment-containing peptide having an amino acid sequence of SEQ ID NO: 40 or 41, and optionally an amino acid substitution of E15K, H16E, H16I, H16V, D20A, D20E, D20K, D20T, S87D, N88A, I92A, I92D, I92E, or I92G.

4. The fusion protein of any one of claims 1 to 3, wherein the IL-2 domain comprises a replacement of the amino acid residues from positions N29 to L40 having an amino acid sequence of SEQ ID NO: 39 with an IL-15 hinge fragment-containing peptide having an amino acid sequence of SEQ ID NO: 40 or 41, and optionally an amino acid substitution of E15K.

5. The fusion protein of any one of claims 1 to 4, wherein the IL-2 domain comprises a replacement of the amino acid residues from positions N29 to L40 having an amino acid sequence of SEQ ID NO: 39 with an IL-15 hinge fragment-containing peptide having an amino acid sequence of SEQ ID NO: 40 or 41, and optionally an amino acid substitution of H16E, H16F, H16I, or H16V.

6. The fusion protein of any one of claims 1 to 5, wherein the IL-2 domain comprises a replacement of the amino acid residues from positions N29 to L40 having an amino acid sequence of SEQ ID NO: 39 with an IL-15 hinge fragment-containing peptide having an amino acid sequence of SEQ ID NO: 40 or 41, and optionally an amino acid substitution of D20A, D20E, D20K, or D20T.

7. The fusion protein of any one of claims 1 to 6, wherein the IL-2 domain comprises a replacement of the amino acid residues from positions N29 to L40 having an amino acid sequence of SEQ ID NO: 39 with an IL-15 hinge fragment-containing peptide having an amino acid sequence of SEQ ID NO: 40 or 41, and optionally an amino acid substitution of S87D.

8. The fusion protein of any one of claims 1 to 7, wherein the IL-2 domain comprises a replacement of the amino acid residues from positions N29 to L40 having an amino acid sequence of SEQ ID NO: 39 with an IL-15 hinge fragment-containing peptide having an amino acid sequence of SEQ ID NO: 40 or 41, and optionally an amino acid substitution of N88A.

9. The fusion protein of any one of claims 1 to 8, wherein the IL-2 domain comprises a replacement of the amino acid residues from positions N29 to L40 having an amino acid sequence of SEQ ID NO: 39 with an IL-15 hinge fragment-containing peptide having an amino acid sequence of SEQ ID NO: 40 or 41, and optionally an amino acid substitution of I92A, I92D, I92E, or I92G.

10. The fusion protein of any one of claims 1 to 9, wherein the IL-2 domain comprises a replacement of the amino acid residues from positions N29 to L40 having an amino acid sequence of SEQ ID NO: 39 with an IL-15 hinge fragment-containing peptide having an amino acid sequence of SEQ ID NO: 40 or 41, and one or two amino acid substitutions of E15K, H16I, D20T, N88A, or I92A.

11. The fusion protein of any one of claims 1 to 10, wherein the IL-2 domain comprises an amino acid substitution at position E15, H16, D20, K32, R38, L40, F42, K76, S87, N88, or I92.

12. The fusion protein of any one of claims 1 to 11, wherein the IL-2 domain comprises an amino acid substitution at position E15, D20, H16, N88, or I92.

13. The fusion protein of any one of claims 1 to 12, wherein the IL-2 domain comprises an amino acid substitution at position E15.

14. The fusion protein of any one of claims 1 to 12, wherein the IL-2 domain comprises an amino acid substitution of E15K.

15. The fusion protein of any one of claims 1 to 14, wherein the IL-2 domain comprises an amino acid substitution at position H16.

16. The fusion protein of any one of claims 1 to 15, wherein the IL-2 domain comprises an amino acid substitution of H16E, H16I, or H16V.

17. The fusion protein of any one of claims 1 to 16, wherein the IL-2 domain comprises an amino acid substitution at position D20.

18. The fusion protein of any one of claims 1 to 17, wherein the IL-2 domain comprises an amino acid substitution of D20A, D20E, D20K, or D20T.

19. The fusion protein of any one of claims 1 to 18, wherein the IL-2 domain comprises an amino acid substitution at position K32.

20. The fusion protein of any one of claims 1 to 19, wherein the IL-2 domain comprises an amino acid substitution of K32E.

21. The fusion protein of any one of claims 1 to 20, wherein the IL-2 domain comprises an amino acid substitution at position R38.

22. The fusion protein of any one of claims 1 to 21, wherein the IL-2 domain comprises an amino acid substitution of R38E or R38N.

23. The fusion protein of any one of claims 1 to 22, wherein the IL-2 domain comprises an amino acid substitution at position L40.

24. The fusion protein of any one of claims 1 to 23, wherein the IL-2 domain comprises an amino acid substitution of L40T.

25. The fusion protein of any one of claims 1 to 24, wherein the IL-2 domain comprises an amino acid substitution at position F42.

26. The fusion protein of any one of claims 1 to 25, wherein the IL-2 domain comprises an amino acid substitution of F42A or F42K.

27. The fusion protein of any one of claims 1 to 26, wherein the IL-2 domain comprises an amino acid substitution at position K76.

28. The fusion protein of any one of claims 1 to 27, wherein the IL-2 domain comprises an amino acid substitution of K76E.

29. The fusion protein of any one of claims 1 to 28, wherein the IL-2 domain comprises an amino acid substitution at position S87.

30. The fusion protein of any one of claims 1 to 29, wherein the IL-2 domain comprises an amino acid substitution of S87D.

31. The fusion protein of any one of claims 1 to 30, wherein the IL-2 domain comprises an amino acid substitution at position N88.

32. The fusion protein of any one of claims 1 to 31, wherein the IL-2 domain comprises an amino acid substitution of N88A.

33. The fusion protein of any one of claims 1 to 32, wherein the IL-2 domain comprises an amino acid substitution at position I92.

34. The fusion protein of any one of claims 1 to 33, wherein the IL-2 domain comprises an amino acid substitution of I92A, I92D, I92E, or I92G.

35. The fusion protein of any one of claims 1 to 34, wherein the IL-2 domain comprises amino acid substitutions at positions R38 and L40 as set forth in SEQ ID NO: 1, 2, 3, 4, or 5.

36. The fusion protein of any one of claims 1 to 35, wherein the IL-2 domain comprises amino acid substitutions of R38N and L40T.

37. The fusion protein of any one of claims 1 to 36, wherein the IL-2 domain comprises amino acid substitutions at positions E15, R38, and L40.

38. The fusion protein of any one of claims 1 to 37, wherein the IL-2 domain comprises amino acid substitutions of E15K, R38N, and L40T.

39. The fusion protein of any one of claims 1 to 38, wherein the IL-2 domain comprises amino acid substitutions at positions E15, K32, R38, and L40.

40. The fusion protein of any one of claims 1 to 39, wherein the IL-2 domain comprises amino acid substitutions of E15K, K32E, R38N, and L40T.

41. The fusion protein of any one of claims 1 to 38, wherein the IL-2 domain comprises amino acid substitutions at positions E15, R38, L40, and K76.

42. The fusion protein of any one of claims 1 to 39 and 41, wherein the IL-2 domain comprises amino acid substitutions of E15K, R38N, L40T, and K76E.

43. The fusion protein of any one of claims 36 to 42, wherein the IL-2 domain is N-glycosylated.

44. The fusion protein of any one of claims 1 to 34, wherein the IL-2 domain comprises amino acid substitutions at positions R38 and F42.

45. The fusion protein of any one of claims 1 to 34, wherein the IL-2 domain comprises amino acid substitutions of R38E and F42A or F42K.

46. The fusion protein of any one of claims 1 to 34, wherein the IL-2 domain comprises amino acid substitutions at positions K32, R38, and F42.

47. The fusion protein of any one of claims 1 to 34, wherein the IL-2 domain comprises amino acid substitutions of K32E, R38E, and F42A or F42K.

48. The fusion protein of any one of claims 1 to 34, wherein the IL-2 domain comprises amino acid substitutions at positions E15, K32, R38, and F42.

49. The fusion protein of any one of claims 1 to 34, wherein the IL-2 domain comprises amino acid substitutions of E15K, K32E, R38E, and F42A or F42K.

50. The fusion protein of any one of claims 1 to 34, wherein the IL-2 domain comprises amino acid substitutions at positions R38, F42, and K76.

51. The fusion protein of any one of claims 1 to 34, wherein the IL-2 domain comprises amino acid substitutions of R38E, F42A or F42K, and K76E.

52. The fusion protein of any one of claims 1 to 34, wherein the IL-2 domain comprises amino acid substitutions at positions E15, R38, F42, and K76.

53. The fusion protein of any one of claims 1 to 34, wherein the IL-2 domain comprises amino acid substitutions of E15K, R38E, F42A or F42K, and K76E.

54. The fusion protein of any one of claims 1 to 53, wherein the interleukin-2 domain comprises a disulfide bond formed between two different amino acid residues from positions N30 to L80.

55. The fusion protein of any one of claims 1 to 54, wherein the interleukin-2 domain comprises a disulfide bond formed between two different amino acid residues, each independently at K35, R38, F42, Y45, E62, V69, or L72.

56. The fusion protein of any one of claims 1 to 55, wherein the interleukin-2 domain comprises a disulfide bond formed between K35C and L72C, R38C and L72C, F42C and V69C, Y42C and L72C, or Y45C and E62C.

57. The fusion protein of any one of claims 1 to 56, wherein the interleukin-2 domain comprises a disulfide bond formed between F42C and V69C.

58. The fusion protein of any one of claims 1 to 57, wherein the interleukin-2 domain comprises a disulfide bond formed between F42C and V69C and an amino acid substitution of E15K, K32E, or K76E.

59. The fusion protein of claim 58, wherein the interleukin-2 domain comprises a disulfide bond formed between F42C and V69C and an amino acid substitution of E15K.

60. The fusion protein of claim 58, wherein the interleukin-2 domain comprises a disulfide bond formed between F42C and V69C and an amino acid substitution of K32E.

61. The fusion protein of claim 58, wherein the interleukin-2 domain comprises a disulfide bond formed between F42C and V69C and an amino acid substitution of K76E.

62. The fusion protein of claim 58, wherein the interleukin-2 domain comprises a disulfide bond formed between F42C and V69C and amino acid substitutions of E15K and K32E.

63. The fusion protein of claim 58, wherein the interleukin-2 domain comprises a disulfide bond formed between F42C and V69C and amino acid substitutions of E15K and K76E.

64. The fusion protein of claim 1, wherein the IL-2 domain comprises an amino acid sequence of any one of SEQ ID NOs: 6 to 37, 42 to 81, 255, and 256.

65. The fusion protein of claim 1, wherein the IL-2 domain comprises an amino acid sequence of SEQ ID NO: 42, 50, 52, 56, 58, 60, 68, 70, 80, 255, or 256.

66. The fusion protein of claim 1, wherein the IL-2 domain comprises an amino acid sequence of SEQ ID NO: 56, 60, 68, 70, 80, 255, or 256.

67. The fusion protein of any one of claims 1 to 66, wherein the PD-1 binding domain comprises:
(i) a CDRL1 of SEQ ID NO: 111, a CDRL2 of DAS, a CDRL3 of SEQ ID NO: 112, a CDRH1 of SEQ ID NO: 113, a CDRH2 of SEQ ID NO: 114, and a CDRH3 of SEQ ID NO: 115;
(ii) a CDRL1 of SEQ ID NO: 120, a CDRL2 of LAS, a CDRL3 of SEQ ID NO: 121, a CDRH1 of SEQ ID NO: 122, a CDRH2 of SEQ ID NO: 123, and a CDRH3 of SEQ ID NO: 124;
(iii) a CDRL1 of SEQ ID NO: 129, a CDRL2 of KVS, a CDRL3 of SEQ ID NO: 130, a CDRH1 of SEQ ID NO: 131, a CDRH2 of SEQ ID NO: 132, and a CDRH3 of SEQ ID NO: 133;
(iv) a CDRL1 of SEQ ID NO: 138, a CDRL2 of TAT, a CDRL3 of SEQ ID NO: 139, a CDRH1 of SEQ ID NO: 140, a CDRH2 of SEQ ID NO: 141, and a CDRH3 of SEQ ID NO: 142;
(v) a CDRL1 of SEQ ID NO: 147, a CDRL2 of AAS, a CDRL3 of SEQ ID NO: 148, a CDRH1 of SEQ ID NO: 149, a CDRH2 of SEQ ID NO: 150, and a CDRH3 of SEQ ID NO: 151;
(vi) a CDRL1 of SEQ ID NO: 156, a CDRL2 of WAS, a CDRL3 of SEQ ID NO: 157, a CDRH1 of SEQ ID NO: 158, a CDRH2 of SEQ ID NO: 159, and a CDRH3 of SEQ ID NO: 160;
(vii) a CDRL1 of SEQ ID NO: 165, a CDRL2 of YAF, a CDRL3 of SEQ ID NO: 166, a CDRH1 of SEQ ID NO: 167, a CDRH2 of SEQ ID NO: 168, and a CDRH3 of SEQ ID NO: 169;
(viii) a CDRL1 of SEQ ID NO: 174, a CDRL2 of AAS, a CDRL3 of SEQ ID NO: 175, a CDRH1 of SEQ ID NO: 176, a CDRH2 of SEQ ID NO: 177, and a CDRH3 of SEQ ID NO: 178;
(ix) a CDRL1 of SEQ ID NO: 183, a CDRL2 of WAS, a CDRL3 of SEQ ID NO: 184, a CDRH1 of SEQ ID NO: 185, a CDRH2 of SEQ ID NO: 186, and a CDRH3 of SEQ ID NO: 187; or
(x) a CDRL1 of SEQ ID NO: 192, a CDRL2 of AAS, a CDRL3 of SEQ ID NO: 193, a CDRH1 of SEQ ID NO: 194, a CDRH2 of SEQ ID NO: 195, and a CDRH3 of SEQ ID NO: 196.

68. The fusion protein of any one of claims 1 to 67, wherein the PD-1 binding domain comprises a CDRL1 of SEQ ID NO: 111, a CDRL2 of DAS, a CDRL3 of SEQ ID NO: 112, a CDRH1 of SEQ ID NO: 113, a CDRH2 of SEQ ID NO: 114, and a CDRH3 of SEQ ID NO: 115.

69. The fusion protein of any one of claims 1 to 67, wherein the PD-1 binding domain comprises a CDRL1 of SEQ ID NO: 120, a CDRL2 of LAS, a CDRL3 of SEQ ID NO: 121, a CDRH1 of SEQ ID NO: 122, a CDRH2 of SEQ ID NO: 123, and a CDRH3 of SEQ ID NO: 124.

70. The fusion protein of any one of claims 1 to 67, wherein the PD-1 binding domain comprises a CDRL1 of SEQ ID NO: 129, a CDRL2 of KVS, a CDRL3 of SEQ ID NO: 130, a CDRH1 of SEQ ID NO: 131, a CDRH2 of SEQ ID NO: 132, and a CDRH3 of SEQ ID NO: 133.

71. The fusion protein of any one of claims 1 to 67, wherein the PD-1 binding domain comprises a CDRL1 of SEQ ID NO: 138, a CDRL2 of TAT, a CDRL3 of SEQ ID NO: 139, a CDRH1 of SEQ ID NO: 140, a CDRH2 of SEQ ID NO: 141, and a CDRH3 of SEQ ID NO: 142.

72. The fusion protein of any one of claims 1 to 67, wherein the PD-1 binding domain comprises a CDRL1 of SEQ ID NO: 147, a CDRL2 of AAS, a CDRL3 of SEQ ID NO: 148, a CDRH1 of SEQ ID NO: 149, a CDRH2 of SEQ ID NO: 150, and a CDRH3 of SEQ ID NO: 151.

73. The fusion protein of any one of claims 1 to 67, wherein the PD-1 binding domain comprises a CDRL1 of SEQ ID NO: 156, a CDRL2 of WAS, a CDRL3 of SEQ ID NO: 157, a CDRH1 of SEQ ID NO: 158, a CDRH2 of SEQ ID NO: 159, and a CDRH3 of SEQ ID NO: 160.

74. The fusion protein of any one of claims 1 to 67, wherein the PD-1 binding domain comprises a CDRL1 of SEQ ID NO: 165, a CDRL2 of YAF, a CDRL3 of SEQ ID NO: 166, a CDRH1 of SEQ ID NO: 167, a CDRH2 of SEQ ID NO: 168, and a CDRH3 of SEQ ID NO: 169.

75. The fusion protein of any one of claims 1 to 67, wherein the PD-1 binding domain comprises a CDRL1 of SEQ ID NO: 174, a CDRL2 of AAS, a CDRL3 of SEQ ID NO: 175, a CDRH1 of SEQ ID NO: 176, a CDRH2 of SEQ ID NO: 177, and a CDRH3 of SEQ ID NO: 178.

76. The fusion protein of any one of claims 1 to 67, wherein the PD-1 binding domain comprises a CDRL1 of SEQ ID NO: 183, a CDRL2 of WAS, a CDRL3 of SEQ ID NO: 184, a CDRH1 of SEQ ID NO: 185, a CDRH2 of SEQ ID NO: 186, and a CDRH3 of SEQ ID NO: 187.

77. The fusion protein of any one of claims 1 to 67, wherein the PD-1 binding domain comprises a CDRL1 of SEQ ID NO: 192, a CDRL2 of AAS, a CDRL3 of SEQ ID NO: 193, a CDRH1 of SEQ ID NO: 194, a CDRH2 of SEQ ID NO: 195, and a CDRH3 of SEQ ID NO: 196.

78. The fusion protein of any one of claims 1 to 77, wherein the half-life-extension domain comprises a fragment crystallizable (Fc) domain.

79. The fusion protein of claim 78, comprising an IL-2 domain, an intact anti-PD-1 antibody comprising two light chains and first and second heavy chains, and optionally a peptide linker; wherein the N-terminus of the IL-2 domain is connected to the C-terminus of the first heavy chains directly or via the peptide linker.

80. The fusion protein of claim 78 or 79, comprising first and second IL-2 domains, an intact anti-PD-1 antibody comprising first and second light chains and first and second heavy chains, and optionally first and second peptide linkers; wherein the N-terminus of the first IL-2 domain is connected to the C-terminus of the first heavy chain directly or via the first peptide linker, and the N-terminus of the second IL-2 domain is connected to the C-terminus of the second heavy chain directly or via the second peptide linker.

81. The fusion protein of claim 79 or 80, wherein the intact anti-PD-1 antibody is a human or humanized antibody.

82. The fusion protein of any one of claims 79 to 81, wherein the intact anti-PD-1 antibody is an IgA, IgD, IgE, IgG, or IgM antibody.

83. The fusion protein of any one of claims 79 to 82, wherein the intact anti-PD-1 antibody is an IgG antibody.

84. The fusion protein of any one of claims 79 to 83, wherein the intact anti-PD-1 antibody is an IgG1 or IgG4 antibody.

85. The fusion protein of any one of claims 79 to 84, wherein the intact anti-PD-1 antibody is an IgG1 antibody.

86. The fusion protein of any one of claims 79 to 84, wherein the intact anti-PD-1 antibody is an IgG4 antibody.

87. The fusion protein of any one of claims 79 to 86, wherein the intact anti-PD-1 antibody is in a knobs-in-holes configuration.

88. The fusion protein of claim 79 or 80, comprising:
(i) a light chain variable region of SEQ ID NO: 116 and a heavy chain variable region of SEQ ID NO: 117;
(ii) a light chain variable region of SEQ ID NO: 125 and a heavy chain variable region of SEQ ID NO: 126;
(iii) a light chain variable region of SEQ ID NO: 134 and a heavy chain variable region of SEQ ID NO: 135;
(iv) a light chain variable region of SEQ ID NO: 143 and a heavy chain variable region of SEQ ID NO: 144;
(v) a light chain variable region of SEQ ID NO: 152 and a heavy chain variable region of SEQ ID NO: 153;
(vi) a light chain variable region of SEQ ID NO: 161 and a heavy chain variable region of SEQ ID NO: 162;
(vii) a light chain variable region of SEQ ID NO: 170 and a heavy chain variable region of SEQ ID NO: 171;
(viii) a light chain variable region of SEQ ID NO: 179 and a heavy chain variable region of SEQ ID NO: 180;
(ix) a light chain variable region of SEQ ID NO: 188 and a heavy chain variable region of SEQ ID NO: 189; or
(x) a light chain variable region of SEQ ID NO: 197 and a heavy chain variable region of SEQ ID NO: 198.

89. The fusion protein of claim 78 or 79, wherein the fusion protein comprises (i) SEQ ID NO: 136; (ii) SEQ ID NO: 248; and (iii) SEQ ID NO: 249, 250, 251, 252, 253, or 254.

90. The fusion protein of claim 78 or 79, wherein the fusion protein comprises (i) SEQ ID NO: 136; (ii) SEQ ID NO: 257; and (iii) SEQ ID NO: 258, 259, 260, 261, 262, or 263.

91. The fusion protein of claim 78 or 79, wherein the fusion protein comprises (i) SEQ ID NO: 136; (ii) SEQ ID NO: 264; and (iii) SEQ ID NO: 202, 265, 266, 267, 268, or 269.

92. The fusion protein of claim 78 or 79, wherein the fusion protein comprises (i) SEQ ID NO: 118; (ii) SEQ ID NO: 201; and (iii) SEQ ID NO: 203 or 204.

93. The fusion protein of any one of claims 78 to 80, where in the fusion protein comprises (i) SEQ ID NO: 118 and (ii) SEQ ID NO: 205.

94. The fusion protein of any one of claims 1 to 77, wherein the half-life-extension domain is an albumin binding domain

95. The fusion protein of claim 94, comprising an IL-2 domain, a PD-1 binding domain, an albumin binding domain, and optionally first and second peptide linkers; wherein the N-terminus of the IL-2 domain is connected to the C-terminus of the albumin binding domain directly or via the first peptide linker, and the N-terminus of the albumin binding domain is connected to the C-terminus of the PD-1 binding domain directly or via the second peptide linker.

96. The fusion protein of claim 94, comprising an IL-2 domain, a PD-1 binding domain, an albumin binding domain, and optionally first and second peptide linkers; wherein the C-terminus of the IL-2 domain is connected to the N-terminus of the albumin binding domain directly or via the first peptide linker, and the C-terminus of the albumin binding domain is connected to the N-terminus of the PD-1 binding domain directly or via the second peptide linker.

97. The fusion protein of claim 94, comprising an IL-2 domain, a PD-1 binding domain, an albumin binding domain, and optionally first and second peptide linkers; wherein the N-terminus of the IL-2 domain is connected to the C-terminus of the PD-1 binding domain directly or via the first peptide linker, and the N-terminus of the PD-1 binding domain is connected to the C-terminus of the albumin binding domain directly or via the second peptide linker.

98. The fusion protein of claim 94, comprising an IL-2 domain, a PD-1 binding domain, an albumin binding domain, and optionally first and second peptide linkers; wherein the C-terminus of the IL-2 domain is connected to the N-terminus of the PD-1 binding domain directly or via the first peptide linker, and the C-terminus of the PD-1 binding domain is connected to the N-terminus of the albumin binding domain directly or via the second peptide linker.

99. The fusion protein of claim 94, comprising an IL-2 domain, a PD-1 binding domain, an albumin binding domain, and optionally first and second peptide linkers; wherein the N-terminus of the PD-1 binding domain is connected to the C-terminus of the IL-2 domain directly or via the first peptide linker, and the N-terminus of the IL-2 domain is connected to the C-terminus of the albumin binding domain directly or via the second peptide linker.

100. The fusion protein of claim 94, comprising an IL-2 domain, a PD-1 binding domain, an albumin binding domain, and optionally first and second peptide linkers; wherein the C-terminus of the PD-1 binding domain is connected to the N-terminus of the IL-2 domain directly or via the first peptide linker, and the C-terminus of the IL-2 domain is connected to the N-terminus of the albumin binding domain directly or via the second peptide linker.

101. The fusion protein of any one of claims 94 to 100, wherein the albumin binding domain is an antibody or a fragment thereof that binds to a human serum albumin.

102. The fusion protein of any one of claims 94 to 101, wherein the albumin binding domain is a single domain antibody.

103. The fusion protein of any one of claims 94 to 102, wherein the albumin binding domain is a V_{H}H single domain antibody.

104. The fusion protein of claim 102 or 103, wherein the single domain antibody comprises (i) CDR1 of SEQ ID NO: 83, CDR2 of SEQ ID NO: 84, and CDR3 of SEQ ID NO: 85; or (ii) CDR1 of SEQ ID NO: 91, CDR2 of SEQ ID NO: 92, and CDR3 of SEQ ID NO: 93.

105. The fusion protein of any one of claims 102 to 104, wherein the single domain antibody has an amino acid sequence of SEQ ID NO: 90 or 97.

106. A pharmaceutical composition comprising the fusion protein of any one of claims 1 to 105, and a pharmaceutically acceptable excipient.

107. The pharmaceutical composition of claim 106, wherein the pharmaceutical composition is in single dosage form.

108. The pharmaceutical composition of claim 106 or 107, wherein the pharmaceutical composition is a solid.

109. The pharmaceutical composition of claim 106 or 107, wherein the pharmaceutical composition is in a parenteral dosage form.

110. The pharmaceutical composition of claim 109, the pharmaceutical composition is in an intravenous dosage form.

111. The pharmaceutical composition of any one of claims 106, 107, 109, or 110, wherein the pharmaceutical composition is a solution.

112. A method of treating one or more symptoms of treating, preventing, or ameliorating one or more symptoms of a proliferative disease in a subject, comprising administering to the subject in need thereof a therapeutically effective amount of the fusion protein of any one of claims 1 to 105 or the pharmaceutical composition of any one of claims 106 to 111.

113. The method of claim 112, wherein the proliferative disease is cancer.

114. The method of claim 112 or 113, wherein the proliferative disease is metastatic cancer.

115. A method of inhibiting the growth of a cell, comprising contacting the cell with an effective amount of the fusion protein of any one of claims 1 to 105 or the pharmaceutical composition of any one of claims 106 to 111.

116. The method of claim 115, wherein the cell is a cancerous cell.

117. A method of activating an immune effector cell, comprising contacting the cell with an effective amount of the fusion protein of any one of claims 1 to 105 or the pharmaceutical composition of any one of claims 106 to 111.


SEQUENCE LISTING (269 sequences)

SEQ ID NO: 1 - protein, 133 aa, homo sapiens [sequence omitted]

SEQ ID NO: 2 - protein, 133 aa, homo sapiens
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 3 - protein, 133 aa, homo sapiens
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 4 - protein, 133 aa, homo sapiens
APASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 5 - protein, 133 aa, homo sapiens
APASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 6 - protein, 133 aa, homo sapiens
APTSSSTKKTQLQLEHLLLDLQMILNGINNYKNPKLTNMTTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 7 - protein, 133 aa, homo sapiens
APASSSTKKTQLQLEHLLLDLQMILNGINNYKNPKLTNMTTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 8 - protein, 133 aa, homo sapiens
APTSSSTKKTQLQLKHLLLDLQMILNGINNYKNPKLTNMTTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 9 - protein, 133 aa, homo sapiens
APASSSTKKTQLQLKHLLLDLQMILNGINNYKNPKLTNMTTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 10 - protein, 133 aa, homo sapiens
APTSSSTKKTQLQLKHLLLDLQMILNGINNYENPKLTNMTTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 11 - protein, 133 aa, homo sapiens
APASSSTKKTQLQLKHLLLDLQMILNGINNYENPKLTNMTTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 12 - protein, 133 aa, homo sapiens
APTSSSTKKTQLQLKHLLLDLQMILNGINNYKNPKLTNMTTFKFYMPKKATELKHLQCLEEELKPLEEVLNLAQSENFHLRPRDLISNINVIVLELKGSETTFMCEYADETATIVEFLNRWITFSQSIISTLT

SEQ ID NO: 13 - protein, 133 aa, homo sapiens
APASSSTKKTQLQLKHLLLDLQMILNGINNYKNPKLTNMTTFKFYMPKKATELKHLQCLEEELKPLEEVLNLAQSENFHLRPRDLISNINVIVLELKGSETTFMCEYADETATIVEFLNRWITFSQSIISTLT

SEQ ID NO: 14 - protein, 133 aa, homo sapiens
APTSSSTKKTQLQLEHLLLDLQMILNGINNYENPKLTEMLTAKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 15 - protein, 133 aa, homo sapiens
APASSSTKKTQLQLEHLLLDLQMILNGINNYENPKLTEMLTAKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 16 - protein, 133 aa, homo sapiens
APTSSSTKKTQLQLEHLLLDLQMILNGINNYENPKLTEMLTKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 17 - protein, 133 aa, homo sapiens
APASSSTKKTQLQLEHLLLDLQMILNGINNYENPKLTEMLTKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 18 - protein, 133 aa, homo sapiens
APTSSSTKKTQLQLKHLLLDLQMILNGINNYENPKLTEMLTKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 19 - protein, 133 aa, homo sapiens
APASSSTKKTQLQLKHLLLDLQMILNGINNYENPKLTEMLTKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 20 - protein, 133 aa, homo sapiens
APTSSSTKKTQLQLEHLLLDLQMILNGINNYKNPKLTEMLTAKFYMPKKATELKHLQCLEEELKPLEEVLNLAQSENFHLRPRDLISNINVIVLELKGSETTFMCEYADETATIVEFLNRWITFSQSIISTLT

SEQ ID NO: 21 - protein, 133 aa, homo sapiens
APASSSTKKTQLQLEHLLLDLQMILNGINNYKNPKLTEMLTAKFYMPKKATELKHLQCLEEELKPLEEVLNLAQSENFHLRPRDLISNINVIVLELKGSETTFMCEYADETATIVEFLNRWITFSQSIISTLT

SEQ ID NO: 22 - protein, 133 aa, homo sapiens
APTSSSTKKTQLQLEHLLLDLQMILNGINNYKNPKLTEMLTKKFYMPKKATELKHLQCLEEELKPLEEVLNLAQSENFHLRPRDLISNINVIVLELKGSETTFMCEYADETATIVEFLNRWITFSQSIISTLT

SEQ ID NO: 23 - protein, 133 aa, homo sapiens
APASSSTKKTQLQLEHLLLDLQMILNGINNYKNPKLTEMLTKKFYMPKKATELKHLQCLEEELKPLEEVLNLAQSENFHLRPRDLISNINVIVLELKGSETTFMCEYADETATIVEFLNRWITFSQSIISTLT

SEQ ID NO: 24 - protein, 133 aa, homo sapiens
APTSSSTKKTQLQLKHLLLDLQMILNGINNYKNPKLTEMLTKKFYMPKKATELKHLQCLEEELKPLEEVLNLAQSENFHLRPRDLISNINVIVLELKGSETTFMCEYADETATIVEFLNRWITFSQSIISTLT

SEQ ID NO: 25 - protein, 133 aa, homo sapiens
APASSSTKKTQLQLKHLLLDLQMILNGINNYKNPKLTEMLTKKFYMPKKATELKHLQCLEEELKPLEEVLNLAQSENFHLRPRDLISNINVIVLELKGSETTFMCEYADETATIVEFLNRWITFSQSIISTLT

SEQ ID NO: 26 - protein, 133 aa, homo sapiens
APTSSSTKKTQLQLEHLLLDLQMILNGINNYKNPKLTRMLTCKFYMPKKATELKHLQCLEEELKPLEECLNLAQSKNFHLRPRDLISNINVIVLELKGSETTFMCEYADETATIVEFLNRWITFSQSIISTLT

SEQ ID NO: 27 - protein, 133 aa, homo sapiens
APASSSTKKTQLQLEHLLLDLQMILNGINNYKNPKLTRMLTCKFYMPKKATELKHLQCLEEELKPLEECLNLAQSKNFHLRPRDLISNINVIVLELKGSETTFMCEYADETATIVEFLNRWITFSQSIISTLT

SEQ ID NO: 28 - protein, 133 aa, homo sapiens
APTSSSTKKTQLQLEHLLLDLQMILNGINNYENPKLTRMLTCKFYMPKKATELKHLQCLEEELKPLEECLNLAQSKNFHLRPRDLISNINVIVLELKGSETTFMCEYADETATIVEFLNRWITFSQSIISTLT

SEQ ID NO: 29 - protein, 133 aa, homo sapiens
APASSSTKKTQLQLEHLLLDLQMILNGINNYENPKLTRMLTCKFYMPKKATELKHLQCLEEELKPLEECLNLAQSKNFHLRPRDLISNINVIVLELKGSETTFMCEYADETATIVEFLNRWITFSQSIISTLT

SEQ ID NO: 30 - protein, 133 aa, homo sapiens
APTSSSTKKTQLQLEHLLLDLQMILNGINNYKNPKLTRMLTCKFYMPKKATELKHLQCLEEELKPLEECLNLAQSENFHLRPRDLISNINVIVLELKGSETTFMCEYADETATIVEFLNRWITFSQSIISTLT

SEQ ID NO: 31 - protein, 133 aa, homo sapiens
APASSSTKKTQLQLEHLLLDLQMILNGINNYKNPKLTRMLTCKFYMPKKATELKHLQCLEEELKPLEECLNLAQSENFHLRPRDLISNINVIVLELKGSETTFMCEYADETATIVEFLNRWITFSQSIISTLT

SEQ ID NO: 32 - protein, 133 aa, homo sapiens
APTSSSTKKTQLQLKHLLLDLQMILNGINNYKNPKLTRMLTCKFYMPKKATELKHLQCLEEELKPLEECLNLAQSKNFHLRPRDLISNINVIVLELKGSETTFMCEYADETATIVEFLNRWITFSQSIISTLT

SEQ ID NO: 33 - protein, 133 aa, homo sapiens
APASSSTKKTQLQLKHLLLDLQMILNGINNYKNPKLTRMLTCKFYMPKKATELKHLQCLEEELKPLEECLNLAQSKNFHLRPRDLISNINVIVLELKGSETTFMCEYADETATIVEFLNRWITFSQSIISTLT

SEQ ID NO: 34 - protein, 133 aa, homo sapiens
APTSSSTKKTQLQLKHLLLDLQMILNGINNYENPKLTRMLTCKFYMPKKATELKHLQCLEEELKPLEECLNLAQSKNFHLRPRDLISNINVIVLELKGSETTFMCEYADETATIVEFLNRWITFSQSIISTLT

SEQ ID NO: 35 - protein, 133 aa, homo sapiens
APASSSTKKTQLQLKHLLLDLQMILNGINNYENPKLTRMLTCKFYMPKKATELKHLQCLEEELKPLEECLNLAQSKNFHLRPRDLISNINVIVLELKGSETTFMCEYADETATIVEFLNRWITFSQSIISTLT

SEQ ID NO: 36 - protein, 133 aa, homo sapiens
APTSSSTKKTQLQLKHLLLDLQMILNGINNYKNPKLTRMLTCKFYMPKKATELKHLQCLEEELKPLEECLNLAQSENFHLRPRDLISNINVIVLELKGSETTFMCEYADETATIVEFLNRWITFSQSIISTLT

SEQ ID NO: 37 - protein, 133 aa, homo sapiens
APASSSTKKTQLQLKHLLLDLQMILNGINNYKNPKLTRMLTCKFYMPKKATELKHLQCLEEELKPLEECLNLAQSENFHLRPRDLISNINVIVLELKGSETTFMCEYADETATIVEFLNRWITFSQSIISTLT

SEQ ID NO: 38 - protein, 22 aa, homo sapiens
NNYKNPKLTRMLTFKFYMPKKA

SEQ ID NO: 39 - protein, 12 aa, homo sapiens
NNYKNPKLTRML

SEQ ID NO: 40 - protein, 8 aa, homo sapiens
QSMEIDAT

SEQ ID NO: 41 - protein, 8 aa, homo sapiens
QSMHIDAT

SEQ ID NO: 42 - protein, 129 aa, homo sapiens
APTSSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 43 - protein, 129 aa, homo sapiens
APASSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 44 - protein, 129 aa, homo sapiens
APTSSSTKKTQLQLEHLLLDLQMILNGIQSMHIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 45 - protein, 129 aa, homo sapiens
APASSSTKKTQLQLEHLLLDLQMILNGIQSMHIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 46 - protein, 129 aa, homo sapiens
APTSSSTKKTQLQLKHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 47 - protein, 129 aa, homo sapiens
APASSSTKKTQLQLKHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 48 - protein, 129 aa, homo sapiens
APTSSSTKKTQLQLKHLLLDLQMILNGIQSMHIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 49 - protein, 129 aa, homo sapiens
APASSSTKKTQLQLKHLLLDLQMILNGIQSMHIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 50 - protein, 129 aa, homo sapiens
APTSSSTKKTQLQLKHLLLDLQMILNGIQSMHIDATTFKFYMPKKATELKHLQCLEEELKPLEEVLNLAQSKNFHLRPRDLIDNINVIVLELKGSETTFMCEYADETATIVEFLNRWITFSQSIISTLT

SEQ ID NO: 51 - protein, 129 aa, homo sapiens
APASSSTKKTQLQLKHLLLDLQMILNGIQSMHIDATTFKFYMPKKATELKHLQCLEEELKPLEEVLNLAQSKNFHLRPRDLIDNINVIVLELKGSETTFMCEYADETATIVEFLNRWITFSQSIISTLT

SEQ ID NO: 52 - protein, 129 aa, homo sapiens
APTSSSTKKTQLQLEELLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 53 - protein, 129 aa, homo sapiens
APASSSTKKTQLQLEELLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 54 - protein, 129 aa, homo sapiens
APTSSSTKKTQLQLEFLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 55 - protein, 129 aa, homo sapiens
APASSSTKKTQLQLEFLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 56 - protein, 129 aa, homo sapiens
APTSSSTKKTQLQLEILLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 57 - protein, 129 aa, homo sapiens
APASSSTKKTQLQLEILLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 58 - protein, 129 aa, homo sapiens
APTSSSTKKTQLQLEVLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 59 - protein, 129 aa, homo sapiens
APASSSTKKTQLQLEVLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 60 - protein, 129 aa, homo sapiens
APTSSSTKKTQLQLEHLLLTLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 61 - protein, 129 aa, homo sapiens
APASSSTKKTQLQLEHLLLTLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 62 - protein, 129 aa, homo sapiens
APTSSSTKKTQLQLEHLLLALQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 63 - protein, 129 aa, homo sapiens
APASSSTKKTQLQLEHLLLALQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 64 - protein, 129 aa, homo sapiens
APTSSSTKKTQLQLEHLLLELQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 65 - protein, 129 aa, homo sapiens
APASSSTKKTQLQLEHLLLELQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 66 - protein, 129 aa, homo sapiens
APTSSSTKKTQLQLEHLLLKLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 67 - protein, 129 aa, homo sapiens
APASSSTKKTQLQLEHLLLKLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 68 - protein, 129 aa, homo sapiens
APTSSSTKKTQLQLEHLLLDLQMILNGIQSMEIDATTFKFYMPKKATELKHLQCLEEELKPLEEVLNLAQSKNFHLRPRDLISAINVIVLELKGSETTFMCEYADETATIVEFLNRWITFSQSIISTLT

SEQ ID NO: 69 - protein, 129 aa, homo sapiens
APASSSTKKTQLQLEHLLLDLQMILNGIQSMEIDATTFKFYMPKKATELKHLQCLEEELKPLEEVLNLAQSKNFHLRPRDLISAINVIVLELKGSETTFMCEYADETATIVEFLNRWITFSQSIISTLT

SEQ ID NO: 70 - protein, 129 aa, homo sapiens
APTSSSTKKTQLQLEHLLLDLQMILNGIQSMEIDATTFKFYMPKKATELKHLQCLEEELKPLEEVLNLAQSKNFHLRPRDLISNINVAVLELKGSETTFMCEYADETATIVEFLNRWITFSQSIISTLT

SEQ ID NO: 71 - protein, 129 aa, homo sapiens
APASSSTKKTQLQLEHLLLDLQMILNGIQSMEIDATTFKFYMPKKATELKHLQCLEEELKPLEEVLNLAQSKNFHLRPRDLISNINVAVLELKGSETTFMCEYADETATIVEFLNRWITFSQSIISTLT

SEQ ID NO: 72 - protein, 129 aa, homo sapiens
APTSSSTKKTQLQLEHLLLDLQMILNGIQSMEIDATTFKFYMPKKATELKHLQCLEEELKPLEEVLNLAQSKNFHLRPRDLISNINVGVLELKGSETTFMCEYADETATIVEFLNRWITFSQSIISTLT

SEQ ID NO: 73 - protein, 129 aa, homo sapiens
APASSSTKKTQLQLEHLLLDLQMILNGIQSMEIDATTFKFYMPKKATELKHLQCLEEELKPLEEVLNLAQSKNFHLRPRDLISNINVGVLELKGSETTFMCEYADETATIVEFLNRWITFSQSIISTLT

SEQ ID NO: 74 - protein, 129 aa, homo sapiens
APTSSSTKKTQLQLEHLLLDLQMILNGIQSMEIDATTFKFYMPKKATELKHLQCLEEELKPLEEVLNLAQSKNFHLRPRDLISNINVDVLELKGSETTFMCEYADETATIVEFLNRWITFSQSIISTLT

SEQ ID NO: 75 - protein, 129 aa, homo sapiens
APASSSTKKTQLQLEHLLLDLQMILNGIQSMEIDATTFKFYMPKKATELKHLQCLEEELKPLEEVLNLAQSKNFHLRPRDLISNINVDVLELKGSETTFMCEYADETATIVEFLNRWITFSQSIISTLT

SEQ ID NO: 76 - protein, 129 aa, homo sapiens
APTSSSTKKTQLQLEHLLLDLQMILNGIQSMEIDATTFKFYMPKKATELKHLQCLEEELKPLEEVLNLAQSKNFHLRPRDLISNINVEVLELKGSETTFMCEYADETATIVEFLNRWITFSQSIISTLT

SEQ ID NO: 77 - protein, 129 aa, homo sapiens
APASSSTKKTQLQLEHLLLDLQMILNGIQSMEIDATTFKFYMPKKATELKHLQCLEEELKPLEEVLNLAQSKNFHLRPRDLISNINVEVLELKGSETTFMCEYADETATIVEFLNRWITFSQSIISTLT

SEQ ID NO: 78 - protein, 129 aa, homo sapiens
APTSSSTKKTQLQLEELLLDLQMILNGIQSMEIDATTFKFYMPKKATELKHLQCLEEELKPLEEVLNLAQSKNFHLRPRDLISNINVAVLELKGSETTFMCEYADETATIVEFLNRWITFSQSIISTLT

SEQ ID NO: 79 - protein, 129 aa, homo sapiens
APASSSTKKTQLQLEELLLDLQMILNGIQSMEIDATTFKFYMPKKATELKHLQCLEEELKPLEEVLNLAQSKNFHLRPRDLISNINVAVLELKGSETTFMCEYADETATIVEFLNRWITFSQSIISTLT

SEQ ID NO: 80 - protein, 129 aa, homo sapiens
APTSSSTKKTQLQLKHLLLDLQMILNGIQSMEIDATTFKFYMPKKATELKHLQCLEEELKPLEEVLNLAQSKNFHLRPRDLISNINVAVLELKGSETTFMCEYADETATIVEFLNRWITFSQSIISTLT

SEQ ID NO: 81 - protein, 129 aa, homo sapiens
APASSSTKKTQLQLKHLLLDLQMILNGIQSMEIDATTFKFYMPKKATELKHLQCLEEELKPLEEVLNLAQSKNFHLRPRDLISNINVAVLELKGSETTFMCEYADETATIVEFLNRWITFSQSIISTLT

SEQ ID NO: 82 - protein, 145 aa, Mus musculus
APTSSSTSSSTAEAQQQQQQQQQQQQHLEQLLMDLQELLSRMQSMEIDATTFKFYLPKQATELKDLQCLEDELGPLRHVLDLTQSKSFQLEDAENFISNIRVTVVKLKGSDNTFECQFDDESATVVDFLRRWIAFSQSIISTSPQ

SEQ ID NO: 83 - protein, 8 aa, Artificial Sequence
  Based on llima sequence
GSTWSINT

SEQ ID NO: 84 - protein, 7 aa, Artificial Sequence
  Based on llima sequence
ISSGGST

SEQ ID NO: 85 - protein, 10 aa, Artificial Sequence
  Based on llima sequence
YAQSTWYPPS

SEQ ID NO: 86 - protein, 25 aa, Homo sapiens [sequence omitted]

SEQ ID NO: 87 - protein, 17 aa, Homo sapiens
LAWYRQAPGKQRDLVAR

SEQ ID NO: 88 - protein, 38 aa, Homo sapiens
YYADSVKGRFTISRDNSKNTLYLQMNSLRAEDTAVYYC

SEQ ID NO: 89 - protein, 11 aa, Homo sapiens
WGQGTLVTVSS

SEQ ID NO: 90 - protein, 116 aa, Artificial Sequence
  Based on llima sequence
EVQLVESGGGLVQPGGSLRLSCAASGSTWSINTLAWYRQAPGKQRDLVARISSGGSTYYADSVKGRFTISRDNSKNTLYLQMNSLRAEDTAVYYCYAQSTWYPPSWGQGTLVTVSS

SEQ ID NO: 91 - protein, 8 aa, Artificial Sequence
  Based on llima sequence
GFAFRGFG

SEQ ID NO: 92 - protein, 8 aa, Artificial Sequence
  Based on llima sequence
INNGGSDT

SEQ ID NO: 93 - protein, 9 aa, Artificial Sequence
  Based on llima sequence
AIGGPGASP

SEQ ID NO: 94 - protein, 25 aa, Homo sapiens
QVQLVESGGGVVQPGGSLRLSCAAS

SEQ ID NO: 95 - protein, 17 aa, Homo sapiens
MSWVRQAPGKGLEWVSS

SEQ ID NO: 96 - protein, 11 aa, Homo sapiens
SGQGTQVTVSS

SEQ ID NO: 97 - protein, 116 aa, Artificial Sequence
  Based on llima sequence
QVQLVESGGGVVQPGGSLRLSCAASGFAFRGFGMSWVRQAPGKGLEWVSSINNGGSDTYYADSVKGRFTISRDNSKNTLYLQMNSLRAEDTAVYYCAIGGPGASPSGQGTQVTVSS

SEQ ID NO: 98 - protein, 330 aa, homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 330 aa, homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 330 aa, homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 330 aa, homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 330 aa, homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 103 - protein, 330 aa, homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 330 aa, homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 105 - protein, 330 aa, homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - protein, 330 aa, homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 107 - protein, 327 aa, homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 108 - protein, 327 aa, homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 109 - protein, 327 aa, homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 110 - protein, 107 aa, homo sapiens [sequence omitted]

SEQ ID NO: 111 - protein, 6 aa, homo sapiens
QSVSSY

SEQ ID NO: 112 - protein, 9 aa, homo sapiens
QQSSNWPRT

SEQ ID NO: 113 - protein, 8 aa, homo sapiens
GITFSNSG

SEQ ID NO: 114 - protein, 8 aa, homo sapiens
IWYDGSKR

SEQ ID NO: 115 - protein, 6 aa, homo sapiens
ATNDDY

SEQ ID NO: 116 - protein, 107 aa, homo sapiens [sequence omitted]

SEQ ID NO: 117 - protein, 113 aa, homo sapiens [sequence omitted]

SEQ ID NO: 118 - protein, 214 aa, homo sapiens
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 440 aa, homo sapiens
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 120 - protein, 10 aa, homo sapiens
KGVSTSGYSY

SEQ ID NO: 121 - protein, 9 aa, homo sapiens
QHSRDLPLT

SEQ ID NO: 122 - protein, 8 aa, homo sapiens
GYTFTNYY

SEQ ID NO: 123 - protein, 8 aa, homo sapiens
INPSNGGT

SEQ ID NO: 124 - protein, 13 aa, homo sapiens
ARRDYRFDMGFDY

SEQ ID NO: 125 - protein, 111 aa, homo sapiens [sequence omitted]

SEQ ID NO: 126 - protein, 120 aa, homo sapiens [sequence omitted]

SEQ ID NO: 127 - protein, 218 aa, homo sapiens
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 128 - protein, 447 aa, homo sapiens
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 129 - protein, 11 aa, homo sapiens
QSIVHSNGNTY

SEQ ID NO: 130 - protein, 9 aa, homo sapiens
FQGSHVPLT

SEQ ID NO: 131 - protein, 8 aa, homo sapiens
GYTFTDYE

SEQ ID NO: 132 - protein, 8 aa, homo sapiens
IESETGGT

SEQ ID NO: 133 - protein, 18 aa, homo sapiens
AREGITTVATTYYWYFDV

SEQ ID NO: 134 - protein, 112 aa, homo sapiens [sequence omitted]

SEQ ID NO: 135 - protein, 125 aa, homo sapiens [sequence omitted]

SEQ ID NO: 136 - protein, 219 aa, homo sapiens
DVVMTQSPLSLPVTLGQPASISCRSSQSIVHSNGNTYLEWYLQKPGQSPQLLIYKVSNRFSGVPDRFSGSGSGTDFTLKISRVEAEDVGVYYCFQGSHVPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 452 aa, homo sapiens
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 138 - protein, 6 aa, homo sapiens
QTIGTW

SEQ ID NO: 139 - protein, 9 aa, homo sapiens
QQVYSIPWT

SEQ ID NO: 140 - protein, 8 aa, homo sapiens
GFTFSSYM

SEQ ID NO: 141 - protein, 8 aa, homo sapiens
ISGGGANT

SEQ ID NO: 142 - protein, 9 aa, homo sapiens
ARQLYYFDY

SEQ ID NO: 143 - protein, 107 aa, homo sapiens
DIQMTQSPSSLSASVGDRVTITCLASQTIGTWLTWYQQKPGKAPKLLIYTATSLADGVPSRFSGSGSGTDFTLTISSLQPEDFATYYCQQVYSIPWTFGGGTKVEIK

SEQ ID NO: 144 - protein, 116 aa, homo sapiens
EVQLVESGGGLVQPGGSLRLSCAASGFTFSSYMMSWVRQAPGKGLEWVATISGGGANTYYPDSVKGRFTISRDNAKNSLYLQMNSLRAEDTAVYYCARQLYYFDYWGQGTTVTVSS

SEQ ID NO: 145 - protein, 214 aa, homo sapiens
DIQMTQSPSSLSASVGDRVTITCLASQTIGTWLTWYQQKPGKAPKLLIYTATSLADGVPSRFSGSGSGTDFTLTISSLQPEDFATYYCQQVYSIPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 146 - protein, 443 aa, homo sapiens
EVQLVESGGGLVQPGGSLRLSCAASGFTFSSYMMSWVRQAPGKGLEWVATISGGGANTYYPDSVKGRFTISRDNAKNSLYLQMNSLRAEDTAVYYCARQLYYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 147 - protein, 6 aa, homo sapiens
QGISSW

SEQ ID NO: 148 - protein, 9 aa, homo sapiens
QQANHLPFT

SEQ ID NO: 149 - protein, 8 aa, homo sapiens
GGTFSSYA

SEQ ID NO: 150 - protein, 8 aa, homo sapiens
IIPMFDTA

SEQ ID NO: 151 - protein, 13 aa, homo sapiens
ARAEHSSTGTFDY

SEQ ID NO: 152 - protein, 107 aa, homo sapiens
DIQMTQSPSSVSASVGDRVTITCRASQGISSWLAWYQQKPGKAPKLLISAASSLQSGVPSRFSGSGSGTDFTLTISSLQPEDFATYYCQQANHLPFTFGGGTKVEIK

SEQ ID NO: 153 - protein, 120 aa, homo sapiens
QVQLVQSGAEVKKPGSSVKVSCKASGGTFSSYAISWVRQAPGQGLEWMGLIIPMFDTAGYAQKFQGRVAITVDESTSTAYMELSSLRSEDTAVYYCARAEHSSTGTFDYWGQGTLVTVSS

SEQ ID NO: 154 - protein, 214 aa, homo sapiens
DIQMTQSPSSVSASVGDRVTITCRASQGISSWLAWYQQKPGKAPKLLISAASSLQSGVPSRFSGSGSGTDFTLTISSLQPEDFATYYCQQANHLPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 155 - protein, 447 aa, homo sapiens
QVQLVQSGAEVKKPGSSVKVSCKASGGTFSSYAISWVRQAPGQGLEWMGLIIPMFDTAGYAQKFQGRVAITVDESTSTAYMELSSLRSEDTAVYYCARAEHSSTGTFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 156 - protein, 12 aa, homo sapiens
QSLLDSGNQKNF

SEQ ID NO: 157 - protein, 9 aa, homo sapiens
QNDYSYPYT

SEQ ID NO: 158 - protein, 8 aa, homo sapiens
GYTFTTYW

SEQ ID NO: 159 - protein, 8 aa, homo sapiens
IYPGTGGS

SEQ ID NO: 160 - protein, 10 aa, homo sapiens
TRWTTGTGAY

SEQ ID NO: 161 - protein, 113 aa, homo sapiens
EIVLTQSPATLSLSPGERATLSCKSSQSLLDSGNQKNFLTWYQQKPGQAPRLLIYWASTRESGVPSRFSGSGSGTDFTETISSLEAEDAATYYCQNDYSYPYTFGQGTKVEIK

SEQ ID NO: 162 - protein, 117 aa, homo sapiens
EVQLVQSGAEVKKPGESLRISCKGSGYTFTTYWMHWVRQATGQGLEWMGNIYPGTGGSNFDEKEKNRVTITADKSTSTAYMELSSLRSEDTAVYYCTRWTTGTGAYWGQGTTVTVSS

SEQ ID NO: 163 - protein, 220 aa, homo sapiens
EIVLTQSPATLSLSPGERATLSCKSSQSLLDSGNQKNFLTWYQQKPGQAPRLLIYWASTRESGVPSRFSGSGSGTDFTETISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 164 - protein, 444 aa, homo sapiens
EVQLVQSGAEVKKPGESLRISCKGSGYTFTTYWMHWVRQATGQGLEWMGNIYPGTGGSNFDEKEKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLQG

SEQ ID NO: 165 - protein, 6 aa, homo sapiens
ESVSND

SEQ ID NO: 166 - protein, 9 aa, homo sapiens
HQAYSSPYT

SEQ ID NO: 167 - protein, 8 aa, homo sapiens
GFSLTSYG

SEQ ID NO: 168 - protein, 7 aa, homo sapiens
IYADGST

SEQ ID NO: 169 - protein, 12 aa, homo sapiens
ARAYGNYWYIDV

SEQ ID NO: 170 - protein, 107 aa, homo sapiens
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIK

SEQ ID NO: 171 - protein, 118 aa, homo sapiens
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSS

SEQ ID NO: 172 - protein, 214 aa, homo sapiens
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 173 - protein, 446 aa, homo sapiens
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQENWYVDGVEVHNAKTKPREEQENSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGIK

SEQ ID NO: 174 - protein, 6 aa, homo sapiens
LSINTF

SEQ ID NO: 175 - protein, 9 aa, homo sapiens
QQSSNTPFT

SEQ ID NO: 176 - protein, 8 aa, homo sapiens
GFTFSNFG

SEQ ID NO: 177 - protein, 8 aa, homo sapiens
ISGGGRDT

SEQ ID NO: 178 - protein, 10 aa, homo sapiens
VKWGNIYFDY

SEQ ID NO: 179 - protein, 107 aa, homo sapiens [sequence omitted]

SEQ ID NO: 180 - protein, 117 aa, homo sapiens [sequence omitted]

SEQ ID NO: 181 - protein, 214 aa, homo sapiens
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 182 - protein, 444 aa, homo sapiens
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 183 - protein, 6 aa, homo sapiens
QDVTTA

SEQ ID NO: 184 - protein, 9 aa, homo sapiens
QQHYTIPWT

SEQ ID NO: 185 - protein, 8 aa, homo sapiens
GFTFSNYG

SEQ ID NO: 186 - protein, 8 aa, homo sapiens
ISGGGSNI

SEQ ID NO: 187 - protein, 9 aa, homo sapiens
VSYYYGIDF

SEQ ID NO: 188 - protein, 107 aa, homo sapiens
DIQMTQSPSSLSASVGDRVTITCKASQDVTTAVAWYQQKPGKAPKLLIYWASTRHTGVPSRFSGSGSGTDFTLTISSLQPEDFATYYCQQHYTIPWTFGGGTKLEIK

SEQ ID NO: 189 - protein, 116 aa, homo sapiens
QVQLVESGGGLVKPGGSLRLSCAASGFTFSNYGMSWIRQAPGKGLEWVSTISGGGSNIYYADSVKGRFTISRDNAKNSLYLQMNSLRAEDTAVYYCVSYYYGIDFWGQGTSVTVSS

SEQ ID NO: 190 - protein, 214 aa, homo sapiens
DIQMTQSPSSLSASVGDRVTITCKASQDVTTAVAWYQQKPGKAPKLLIYWASTRHTGVPSRFSGSGSGTDFTLTISSLQPEDFATYYCQQHYTIPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 191 - protein, 443 aa, homo sapiens
QVQLVESGGGLVKPGGSLRLSCAASGFTFSNYGMSWIRQAPGKGLEWVSTISGGGSNIYYADSVKGRFTISRDNAKNSLYLQMNSLRAEDTAVYYCVSYYYGIDFWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 192 - protein, 10 aa, homo sapiens
ESVDNYGMSF

SEQ ID NO: 193 - protein, 9 aa, homo sapiens
QQSKEVPYT

SEQ ID NO: 194 - protein, 8 aa, homo sapiens
GYSFTSYW

SEQ ID NO: 195 - protein, 8 aa, homo sapiens
IHPSDSET

SEQ ID NO: 196 - protein, 12 aa, homo sapiens
AREHYGTSPFAY

SEQ ID NO: 197 - protein, 111 aa, homo sapiens
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIK

SEQ ID NO: 198 - protein, 119 aa, homo sapiens
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSS

SEQ ID NO: 199 - protein, 218 aa, homo sapiens
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 200 - protein, 445 aa, homo sapiens
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 201 - protein, 440 aa, Homo sapiens
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 202 - protein, 596 aa, homo sapiens
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSGGGGSGGGGSAPTSSSTKKTQLQLKHLLLDLQMILNGIQSMEIDATTFKFYMPKKATELKHLQCLEEELKPLEEVLNLAQSKNFHLRPRDLISNINVAVLELKGSETTFMCEYADETATIVEFLNRWITFSQSIISTLT

SEQ ID NO: 203 - protein, 584 aa, Homo sapiens
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSAPTSSSTKKTQLQLEHLLLDLQMILNGIQSMEIDATTFKFYMPKKATELKHLQCLEEELKPLEEVLNLAQSKNFHLRPRDLISNINVAVLELKGSETTFMCEYADETATIVEFLNRWITFSQSIISTLT

SEQ ID NO: 204 - protein, 584 aa, Homo sapiens
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSAPTSSSTKKTQLQLKHLLLDLQMILNGIQSMEIDATTFKFYMPKKATELKHLQCLEEELKPLEEVLNLAQSKNFHLRPRDLISAINVIVLELKGSETTFMCEYADETATIVEFLNRWITFSQSIISTLT

SEQ ID NO: 205 - protein, 584 aa, Homo sapiens
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSAPTSSSTKKTQLQLKHLLLDLQMILNGIQSMEIDATTFKFYMPKKATELKHLQCLEEELKPLEEVLNLAQSKNFHLRPRDLISAINVIVLELKGSETTFMCEYADETATIVEFLNRWITFSQSIISTLT

SEQ ID NO: 206 - protein, 6 aa, Artificial Sequence
  Linker
GSGGSG

SEQ ID NO: 207 - protein, 9 aa, Artificial Sequence
  Linker
GSGGSGGSG

SEQ ID NO: 208 - protein, 12 aa, Artificial Sequence
  Linker
GSGGSGGSGGSG

SEQ ID NO: 209 - protein, 4 aa, Artificial Sequence
  Linker
GGGS

SEQ ID NO: 210 - protein, 8 aa, Artificial Sequence
  Linker
GGGSGGGS

SEQ ID NO: 211 - protein, 12 aa, Artificial Sequence
  Linker
GGGSGGGSGGGS

SEQ ID NO: 212 - protein, 16 aa, Artificial Sequence
  Linker
GGGSGGGSGGGSGGGS

SEQ ID NO: 213 - protein, 5 aa, Artificial Sequence
  Linker
GGGGS

SEQ ID NO: 214 - protein, 10 aa, Artificial Sequence
  Linker
GGGGSGGGGS

SEQ ID NO: 215 - protein, 15 aa, Artificial Sequence
  Linker
GGGGSGGGGSGGGGS

SEQ ID NO: 216 - protein, 20 aa, Artificial Sequence
  Linker
GGGGSGGGGSGGGGSGGGGS

SEQ ID NO: 217 - protein, 4 aa, Artificial Sequence
  Linker
SGSG

SEQ ID NO: 218 - protein, 7 aa, Artificial Sequence
  Linker
SGSGGSG

SEQ ID NO: 219 - protein, 10 aa, Artificial Sequence
  Linker
SGSGGSGGSG

SEQ ID NO: 220 - protein, 13 aa, Artificial Sequence
  Linker
SGSGGSGGSGGSG

SEQ ID NO: 221 - protein, 5 aa, Artificial Sequence
  Linker
SGGGS

SEQ ID NO: 222 - protein, 9 aa, Artificial Sequence
  Linker
SGGGSGGGS

SEQ ID NO: 223 - protein, 13 aa, Artificial Sequence
  Linker
SGGGSGGGSGGGS

SEQ ID NO: 224 - protein, 17 aa, Artificial Sequence
  Linker
SGGGSGGGSGGGSGGGS

SEQ ID NO: 225 - protein, 6 aa, Artificial Sequence
  Linker
SGGGGS

SEQ ID NO: 226 - protein, 11 aa, Artificial Sequence
  Linker
SGGGGSGGGGS

SEQ ID NO: 227 - protein, 16 aa, Artificial Sequence
  Linker
SGGGGSGGGGSGGGGS

SEQ ID NO: 228 - protein, 21 aa, Artificial Sequence
  Linker
SGGGGSGGGGSGGGGSGGGGS

SEQ ID NO: 229 - protein, 5 aa, Artificial Sequence
  Linker
EAAAK

SEQ ID NO: 230 - protein, 10 aa, Artificial Sequence
  Linker
EAAAKEAAAK

SEQ ID NO: 231 - protein, 15 aa, Artificial Sequence
  Linker
EAAAKEAAAKEAAAK

SEQ ID NO: 232 - protein, 20 aa, Artificial Sequence
  Linker
EAAAKEAAAKEAAAKEAAAK

SEQ ID NO: 233 - protein, 5 aa, Artificial Sequence
  Linker
PAPAP

SEQ ID NO: 234 - protein, 10 aa, Artificial Sequence
  Linker
PAPAPPAPAP

SEQ ID NO: 235 - protein, 15 aa, Artificial Sequence
  Linker
PAPAPPAPAPPAPAP

SEQ ID NO: 236 - protein, 20 aa, Artificial Sequence
  Linker
PAPAPPAPAPPAPAPPAPAP

SEQ ID NO: 237 - protein, 8 aa, Artificial Sequence
  Linker
IKRTVAAP

SEQ ID NO: 238 - protein, 5 aa, Artificial Sequence
  Linker
RAKPS

SEQ ID NO: 239 - protein, 10 aa, Artificial Sequence
  Linker
RAKPSRAKPS

SEQ ID NO: 240 - protein, 5 aa, Artificial Sequence
  Linker
ASTKG

SEQ ID NO: 241 - protein, 10 aa, Artificial Sequence
  Linker
ASTKGASTKG

SEQ ID NO: 242 - protein, 5 aa, Artificial Sequence
  Linker
AKTHT

SEQ ID NO: 243 - protein, 10 aa, Artificial Sequence
  Linker
AKTHTAKTHT

SEQ ID NO: 244 - protein, 15 aa, Artificial Sequence
  Linker
ASTKGGGGGSGGGGS

SEQ ID NO: 245 - protein, 15 aa, Artificial Sequence
  Linker
RAKPSGGGGSGGGGS

SEQ ID NO: 246 - protein, 257 aa, homo sapiens
APTSSSTKKTQLQLKHLLLDLQMILNGIQSMHIDATTFKFYMPKKATELKHLQCLEEELKPLEEVLNLAQSKNFHLRPRDLIDNINVIVLELKGSETTFMCEYADETATIVEFLNRWITFSQSIISTLTGSGGSGGSGGSGEVQLVESGGGLVQPGGSLRLSCAASGSTWSINTLAWYRQAPGKQRDLVARISSGGSTYYADSVKGRFTISRDNSKNTLYLQMNSLRAEDTAVYYCYAQSTWYPPSWGQGTLVTVSS

SEQ ID NO: 247 - protein, 455 aa, homo sapiens
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 248 - protein, 455 aa, homo sapiens
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGA

SEQ ID NO: 249 - protein, 599 aa, homo sapiens
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGGGGSAPTSSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 250 - protein, 599 aa, homo sapiens
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGGGGSAPTSSSTKKTQLQLEELLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 251 - protein, 599 aa, homo sapiens
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGGGGSAPTSSSTKKTQLQLEHLLLDLQMILNGIQSMEIDATTFKFYMPKKATELKHLQCLEEELKPLEEVLNLAQSKNFHLRPRDLISAINVIVLELKGSETTFMCEYADETATIVEFLNRWITFSQSIISTLT

SEQ ID NO: 252 - protein, 599 aa, homo sapiens
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGGGGSAPTSSSTKKTQLQLKHLLLDLQMILNGIQSMEIDATTFKFYMPKKATELKHLQCLEEELKPLEEVLNLAQSKNFHLRPRDLISNINVAVLELKGSETTFMCEYADETATIVEFLNRWITFSQSIISTLT

SEQ ID NO: 253 - protein, 599 aa, homo sapiens
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGGGGSAPTSSSTKKTQLQLEHLLLDLQMILNGIQSMEIDATTFKFYMPKKATELKHLQCLEEELKPLEEVLNLAQSKNFHLRPRDLISNINVAVLELKGSETTFMCEYADETATIVEFLNRWITFSQSIISTLT

SEQ ID NO: 254 - protein, 599 aa, homo sapiens
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGGGGSAPTSSSTKKTQLQLKHLLLDLQMILNGIQSMHIDATTFKFYMPKKATELKHLQCLEEELKPLEEVLNLAQSKNFHLRPRDLIDNINVIVLELKGSETTFMCEYADETATIVEFLNRWITFSQSIISTLT

SEQ ID NO: 255 - protein, 129 aa, homo sapiens
APTSSSTKKTQLQLKILLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 256 - protein, 129 aa, homo sapiens
APTSSSTKKTQLQLKHLLLDLQMILNGIQSMEIDATTFKFYMPKKATELKHLQCLEEELKPLEEVLNLAQSKNFHLRPRDLISAINVIVLELKGSETTFMCEYADETATIVEFLNRWITFSQSIISTLT

SEQ ID NO: 257 - protein, 452 aa, homo sapiens
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGA

SEQ ID NO: 258 - protein, 596 aa, homo sapiens
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSGGGGSGGGGSAPTSSSTKKTQLQLEILLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 259 - protein, 596 aa, homo sapiens
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSGGGGSGGGGSAPTSSSTKKTQLQLKILLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 260 - protein, 596 aa, homo sapiens
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSGGGGSGGGGSAPTSSSTKKTQLQLEHLLLDLQMILNGIQSMEIDATTFKFYMPKKATELKHLQCLEEELKPLEEVLNLAQSKNFHLRPRDLISAINVIVLELKGSETTFMCEYADETATIVEFLNRWITFSQSIISTLT

SEQ ID NO: 261 - protein, 596 aa, homo sapiens
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSGGGGSGGGGSAPTSSSTKKTQLQLKHLLLDLQMILNGIQSMEIDATTFKFYMPKKATELKHLQCLEEELKPLEEVLNLAQSKNFHLRPRDLISAINVIVLELKGSETTFMCEYADETATIVEFLNRWITFSQSIISTLT

SEQ ID NO: 262 - protein, 596 aa, homo sapiens
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSGGGGSGGGGSAPTSSSTKKTQLQLEHLLLDLQMILNGIQSMEIDATTFKFYMPKKATELKHLQCLEEELKPLEEVLNLAQSKNFHLRPRDLISNINVAVLELKGSETTFMCEYADETATIVEFLNRWITFSQSIISTLT

SEQ ID NO: 263 - protein, 596 aa, homo sapiens
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSGGGGSGGGGSAPTSSSTKKTQLQLKHLLLDLQMILNGIQSMEIDATTFKFYMPKKATELKHLQCLEEELKPLEEVLNLAQSKNFHLRPRDLISNINVAVLELKGSETTFMCEYADETATIVEFLNRWITFSQSIISTLT

SEQ ID NO: 264 - protein, 452 aa, homo sapiens
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGA

SEQ ID NO: 265 - protein, 596 aa, homo sapiens
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSGGGGSGGGGSAPTSSSTKKTQLQLEILLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 266 - protein, 596 aa, homo sapiens
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSGGGGSGGGGSAPTSSSTKKTQLQLKILLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 267 - protein, 596 aa, homo sapiens
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSGGGGSGGGGSAPTSSSTKKTQLQLEHLLLDLQMILNGIQSMEIDATTFKFYMPKKATELKHLQCLEEELKPLEEVLNLAQSKNFHLRPRDLISAINVIVLELKGSETTFMCEYADETATIVEFLNRWITFSQSIISTLT

SEQ ID NO: 268 - protein, 596 aa, homo sapiens
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSGGGGSGGGGSAPTSSSTKKTQLQLKHLLLDLQMILNGIQSMEIDATTFKFYMPKKATELKHLQCLEEELKPLEEVLNLAQSKNFHLRPRDLISAINVIVLELKGSETTFMCEYADETATIVEFLNRWITFSQSIISTLT

SEQ ID NO: 269 - protein, 596 aa, homo sapiens
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSGGGGSGGGGSAPTSSSTKKTQLQLEHLLLDLQMILNGIQSMEIDATTFKFYMPKKATELKHLQCLEEELKPLEEVLNLAQSKNFHLRPRDLISNINVAVLELKGSETTFMCEYADETATIVEFLNRWITFSQSIISTLT